(19) United States
(12) Patent Application Publication
(10) Pub. No.: US 2016/0264670 A1
(43) Pub. Date: Sep. 15, 2016

(54) IMMUNOTHERAPEUTIC DOSING REGIMENS AND COMBINATIONS THEREOF

(71) Applicant: BRISTOL-MYERS SQUIBB COMPANY, Princeton, NJ (US)

(72) Inventors: Robert F Graziano, Frenchtown, NJ (US); Michael Darron Robbins, Hillsborough, NJ (US); Maria Jure-Kunkel, Plainsboro, NJ (US)

(21) Appl. No.: 15/035,080

(22) PCT Filed: Nov. 5, 2014
(86) PCT No.: PCT/US2014/064036

§ 371 Date: May 6, 2016

Related U.S. Application Data
(60) Provisional application No. 62/029,001, filed on Jul. 25, 2014.
(60) Provisional application No. 61/911,669, filed on Dec. 4, 2013.
(60) Provisional application No. 61/900,646, filed on Nov. 6, 2013.

(51) Int. Cl. C07K 16/28

(52) U.S. Cl. CPC C07K 16/2806; C07K 16/2896; C07K 2317/21; C07K 2317/565; A61K 2039/507

(57) ABSTRACT

The invention described herein relates to therapeutic dosing regimens and combinations thereof for use in enhancing the therapeutic efficacy of anti-CS1 antibodies in combination with one or more immunotherapeutic agents.

This application claims benefit to provisional application U.S. Ser. No. 61/900,646, filed Nov. 6, 2013; to provisional application U.S. Ser. No. 61/911,669, filed Dec. 4, 2013; and to provisional application U.S. Ser. No. 62/029,001, filed Jul. 25, 2014; under 35 U.S.C. §119(e). The entire teachings of the referenced applications are incorporated herein by reference.

### FIELD OF THE INVENTION

The invention described herein relates to therapeutic dosing regimens and combinations thereof for use in enhancing the therapeutic efficacy of anti-CS1 antibodies in combination with one or more immunotherapeutic agents.

### BACKGROUND OF THE INVENTION

The National Cancer Institute has estimated that in the United States alone, 1 in 3 people will be struck with cancer during their lifetime. Moreover, approximately 50% to 60% of people contracting cancer will eventually succumb to the disease. The widespread occurrence of this disease underscores the need for improved anticancer regimens for the treatment of malignancy.

Cancer can occur in any tissue or organ of the body. Plasma cell neoplasms, including multiple myeloma, “Solitary” myeloma of bone, extramedullary plasmacytoma, plasma cell leukemia, macroglobulinemia (including Waldenstrom's macroglobulinemia), heavy-chain disease, primary amyloidosis, monoclonal gammopathy of unknown significance (MGUS) are associated with increased expression of immunoglobulins. Chronic lymphocytic leukemia (CLL), a non-plasma cell neoplasm, is also associated with high levels of immunoglobulin expression.

Increased expression of immunoglobulin can also be seen in malignant diseases. Like autoimmune disorders, the etiology of cancer is similarly multi-factorial in origin. Cancer, which is the second leading cause of death in the United States, has been linked to mutations in DNA that cause unrestrained growth of cells. Genetic predisposition plays a large role in the development of many cancers, combined with environmental factors, such as smoking and chemical mutagenesis.

Myelomas are tumors of plasma cells derived from a single clone, which typically originates in secondary lymphoid tissue and then migrates into and resides in bone marrow tissue. Myelomas commonly affect the bone marrow and adjacent bone structures, with primary symptoms of bone pain and pathological fractures or lesions (osteolytic bone lesions), abnormal bleeding, anemia and increased susceptibility to infections. Advanced stages of the disease include renal failure, skeletal deformities, compaction of the spinal cord, and hypercalcemia. Myeloma affects bone cells by inducing osteoclast resorption of bone, hence decimating bone structure and increasing calcium concentration in plasma. The etiology of myelomas is currently unknown. Linkage to radiation damage, mutations in oncogenes, familial causes and abnormal IL6 expression have been postulated.

Multiple myelomas are plasma cell tumors with multiple origins. Multiple myelomas account for nearly 10% of all plasma cell malignancies, and are the most common bone tumor cancer in adults, with an annual incident rate of 3 to 4 cases per 100,000 people with a median age at diagnosis of between 63 and 70 years. In the United States, multiple myelomas are the second most common hematologic malignancy after Non-Hodgkin's Lymphoma, with approximately 50,000 cases in the United States alone, and approximately 13,500 new reported cases every year. In Europe, the incidence of multiple myelomas is 6 cases per 100,000 people per year. The prognosis outlook for patients diagnosed with multiple myelomas is grim, with only several months to a year for patients with advanced forms of the disease.

Traditional treatment regions for myeloma and multiple myelomas (henceforth referred to as “myeloma”) consist of chemotherapy, radiation therapy, and surgery. In addition, bone marrow transplantation is recommended for patients who are otherwise in good health. The cure rate for patients approaches 30%, and is the only method known that can cure myelomas. However, for individuals who are older or cannot tolerate bone marrow transplantation procedures, chemotherapy is most appropriate.

Recently, important advances in multiple myeloma therapies such as the introduction of autologous stem cell transplantation (ASCT) and the availability of thalidomide, lenalidomide (immunomodulatory drugs or IMiDs) and bortezomib have changed the management of these patients and have allowed an increase in overall survival (OS) (Kristinsson et al., J. Clin. Oncol., 25:1993-1999 (2007); Brenner et al., Blood, 111:2521-2526 (2008); and Kumar et al., Blood, 111:2516-2520 (2008)). Patients younger than 60 years have a 10 year survival probability of ˜30% (Raab et al., Lancet, 374:324-339 (2009)). Thalidomide (Rajkumar et al., J. Clin. Oncol., 26:2171-2177 (2008)), lenalidomide (Rajkumar et al., Lancet Oncol., 11:29-37 (2010)); or bortezomib (Harousseau et al., J. Clin. Oncol., 28:4621-4629 (2010)), in combination with dexamethasone as part of an induction therapy regimen before ASCT has led to rates of nearly CR of 8, 15 and 16%, respectively; whereas three-drug induction schedules of bortezomib-dexamethasone plus doxorubicin (Sonneveld et al., Blood (ASH Annual Meeting Abstracts), 116:23 (2010)), cyclophosphamide (Reeder et al., Leukemia, 23:1337-1341 (2009)), thalidomide (Cavo et al., Lancet, 376:2075-2085 (2010)); or lenalidomide (Richardson et al., Blood, 116:679-686 (2010)), permits achievement rates of nearly CR of 7, 39, 32 and 57%, respectively. However, despite these advances, almost all multiple myeloma patients relapse.

The appearance of abnormal antibodies, known as M-protein, is a diagnostic indicator of multiple myeloma. The increased production of M-protein has been linked to hyperviscosity syndrome in multiple myelomas, causing debilitating side effects, including fatigue, headaches, shortness of breath, mental confusion, chest pain, kidney damage and failure, vision problems and Raynaud's phenomenon (poor blood circulation, particularly fingers, toes, nose and ears). Cryoglobulinemia occurs when M-protein in the blood forms particles under cold conditions. These particles can block small blood vessels and cause pain and numbness in the toes, fingers, and other extremities during cold weather. Prognostic indicators, such as chromosomal deletions, elevated levels of beta-2 microglobulin, serum creatinine levels and IgA isotyping have also been studied. Tricot G. et al., “Poor Prognosis in Multiple Myeloma”, Blood, 86:4250-4252 (1995).

Immunostimulatory monoclonal antibodies (mAb) represent a new and exciting strategy in cancer immunotherapy to potentiate the immune responses of the host against the malignancy (Melero et al., Nat. Rev. Cancer, 7:95-106 (2007)). Such agonistic or antagonistic mAbs bind to key receptors in cells of the immune system acting to enhance antigen presentation (e.g., anti-CD40), to provide costimulation (e.g., anti-CD137), or to counteract immunoregulation (e.g., anti-CTLA-4).

CD137 (also called 4-1BB) is a T-cell costimulatory receptor induced on TCR activation (Nam et al., Curr. Cancer Drug Targets, 5:357-363 (2005); Watts et al., Annu. Rev. Immunol., 23:23-68 (2005)). In addition to its expression on activated CD4+ and CD8+ T cells, CD137 is also expressed on CD4+CD25+ regulatory T cells, activated natural killer (NK) and NK-T cells, monocytes, neutrophils, and dendritic cells. Its natural ligand, CD137L, has been described on antigen-presenting cells including B cells, monocyte/macrophages, and dendritic cells (Watts et al., Annu. Rev. Immunol., 23:23-68 (2005)). On interaction with its ligand, CD137 leads to increased TCR-induced T-cell proliferation, cytokine production, functional maturation, and prolonged CD8+ T-cell survival (Nam et al., Curr. Cancer Drug Targets, 5:357-363 (2005), Watts et al., Annu. Rev. Immunol., 23:23-68 (2005)).

Urelumab is a fully human agonistic monoclonal antibody targeting the CD137 receptor with potential immunostimulatory and antineoplastic activities. Urelumab specifically binds to and activates CD137-expressing immune cells, stimulating an immune response, in particular a cytotoxic T cell response, against tumor cells. CD137 is a member of the tumor necrosis factor (TNF)/nerve growth factor (NGF) family of receptors. Urelumab is currently being evaluated in combination with Rituximab in a Phase 1 trial for the treatment of Non-Hodgkins Lymphoma or CLL.

CS1 (also known as SLAMF7, CRACC, 19A, APEX-1, FOAP12, and 19A; GENBANK® Accession No. NM_021181.3, Ref. Boles et al., Immunogenetics, 52:302-307 (2001); Bouchon et al., J. Immunol., 167:5517-5521 (2001); Murphy et al., Biochem. J., 361:431-436 (2002)) is a member of the CD2 subset of the immunoglobulin superfamily. Molecules of the CD2 family are involved in a broad range of immunomodulatory functions, such as co-activation, proliferation differentiation, and adhesion of lymphocytes, as well as immunoglobulin secretion, cytokine production, and NK cell cytotoxicity. Several members of the CD2 family, such as CD2, CD58, and CD150, play a role or have been proposed to play a role in a number of autoimmune and inflammatory diseases, such as psoriasis, rheumatoid arthritis, and multiple sclerosis. It has been reported that CS1 plays a role in NK cell-mediated cytotoxicity and lymphocyte adhesion (Bouchon, A. et al., J. Immunol., 5517-5521 (2001); Murphy, J. et al., Biochem. 1, 361:431-436 (2002)).

Elotuzumab is a humanized monoclonal IgG1 antibody directed against CS-1, a cell surface glycoprotein, which is highly and uniformly expressed in multiple myeloma. Elotuzumab induces significant antibody-dependent cellular cytotoxicity (ADCC) against primary multiple myeloma cells in the presence of peripheral lymphocytes (Tai et al., Blood, 112:1329-1337 (2008)). Results of three studies that evaluated the safety and efficacy of this drug administered alone (Zonder et al., Blood, 120(3):552-559 (2012)), in combination with bortezomib (Jakubowiak et al., J. Clin. Oncol., 30(16):1960-1965 (Jun. 1, 2012)), or lenalidomide and low-dose dexamethasone (Lonial et al., J. Clin. Oncol., 30:1953-1959 (2012); and Richardson et al., Blood (ASH Annual Meeting Abstracts) 116:986 (2010) for the treatment of patients with relapsed or refractory multiple myeloma, have been reported. All three combinations showed a manageable safety profile and encouraging activity. For example, a Phase I/II study evaluating the safety and efficacy of elotuzumab in combination lenalidomide and low-dose dexamethasone for the treatment of relapsed or refractory multiple myeloma demonstrated a 33 month PFS as well as a 92% response rate for patients receiving the 10 mg/kg dose (Lonial et al., J. Clin. Oncol., 31 (2013) (Suppl., Abstr. 8542)). Phase III clinical trials of lenalidomide/dexamethasone with or without elotuzumab in previously untreated multiple myeloma patients is ongoing, while another phase III trial designed to evaluate this same combination in the first line setting is also ongoing.

The present inventors have discovered, for the first time, that administration of a therapeutically effective amount of an agonistic CD137 antibody with a therapeutically effective amount of an anti-CS1 antibody, results in synergistic regressions of multiple myeloma cells and tumors, thus establishing this combination as a potential treatment option for multiple myeloma patients.

### SUMMARY OF THE INVENTION

The present invention provides a method for treating a patient with multiple myeloma comprising the concurrent administration of a combination therapeutic regiment comprising: (i) a therapeutically effective amount of an agonistic CD137 antibody; and (ii) a therapeutically effective amount of an anti-CS1 antibody, wherein said combination results in the synergistic reduction in tumor burden, tumor regression, and/or tumor development of said cancer.

The present invention provides a method for treating a patient with cancer comprising the concurrent administration of a combination therapeutic regiment comprising: (i) a therapeutically effective amount of an agonistic CD137 antibody; and (ii) a therapeutically effective amount of an anti-CS1 antibody, wherein said combination results in the synergistic reduction in tumor burden, tumor regression, and/or tumor development of said cancer, wherein said cancer is selected from the group consisting of: myeloma, multiple myeloma, and smoldering myeloma, among others.

The present invention provides a method for treating a patient with multiple myeloma comprising the concurrent administration of a combination therapeutic regiment comprising: (i) a therapeutically effective amount of an agonistic CD137 antibody; and (ii) a therapeutically effective amount of an anti-CS1 antibody, wherein said combination results in the synergistic reduction in tumor burden, tumor regression, and/or tumor development of said cancer, wherein said anti-CS1 antibody is elotuzumab.

The present invention provides a method for treating a patient with cancer comprising the concurrent administration of a combination therapeutic regiment comprising: (i) a therapeutically effective amount of an agonistic CD137 antibody; and (ii) a therapeutically effective amount of an anti-CS1 antibody, wherein said combination results in the synergistic reduction in tumor burden, tumor regression, and/or tumor development of said cancer, wherein said cancer is selected from the group consisting of: melanoma, multiple myeloma, smoldering myeloma, and wherein said anti-CS1 antibody is elotuzumab.

The present invention provides a method for treating a patient with multiple myeloma comprising the concurrent administration of a combination therapeutic regiment comprising: (i) a therapeutically effective amount of an agonistic CD137 antibody; and (ii) a therapeutically effective amount of an anti-CS1 antibody, wherein said combination results in the synergistic reduction in tumor burden, tumor regression, and/or tumor development of said cancer, and wherein said CD137 antibody is urelumab.

The present invention provides a method for treating a patient with cancer comprising the concurrent administration of a combination therapeutic regiment comprising: (i) a therapeutically effective amount of an agonistic CD137 antibody; and (ii) a therapeutically effective amount of an anti-CS1 antibody, wherein said combination results in the synergistic reduction in tumor burden, tumor regression, and/or tumor development of said cancer, wherein said cancer is selected from the group consisting of: myeloma, multiple myeloma, smoldering myeloma, and wherein said CD137 antibody is urelumab.

The present invention provides a method for treating a patient with multiple myeloma comprising the concurrent administration of a combination therapeutic regiment comprising: (i) a therapeutically effective amount of an agonistic CD137 antibody; and (ii) a therapeutically effective amount of an anti-CS1 antibody, wherein said combination results in the synergistic reduction in tumor burden, tumor regression, and/or tumor development of said cancer, wherein said anti-CS1 antibody is elotuzumab, and wherein said CD137 antibody is urelumab.

The present invention provides a method for treating a patient with cancer comprising the concurrent administration of a combination therapeutic regiment comprising: (i) a therapeutically effective amount of an agonistic CD137 antibody; and (ii) a therapeutically effective amount of an anti-CS1 antibody, wherein said combination results in the synergistic reduction in tumor burden, tumor regression, and/or tumor development of said cancer, wherein said cancer is selected from the group consisting of: myeloma, multiple myeloma, smoldering myeloma, wherein said anti-CS1 antibody is elotuzumab, and wherein said CD137 antibody is urelumab.

The present invention provides a method for treating a patient with cancer comprising the concurrent administration of a combination therapeutic regiment comprising: (i) a therapeutically effective amount of an agonistic CD137 antibody; and (ii) a therapeutically effective amount of an anti-CS1 antibody, wherein said combination results in the synergistic reduction in tumor burden, tumor regression, and/or tumor development of said cancer, wherein said cancer is selected from the group consisting of: myeloma, multiple myeloma, smoldering myeloma, wherein said anti-CS1 antibody is elotuzumab, wherein said CD137 antibody is urelumab, wherein said agonistic CD137 antibody is administered at a dosage of about 0.03-1 mg/kg, or about 3 mg-8 mg.

The present invention provides a method for treating a patient with cancer comprising the concurrent administration of a combination therapeutic regiment comprising: (i) a therapeutically effective amount of an agonistic CD137 antibody; and (ii) a therapeutically effective amount of an anti-CS1 antibody, wherein said combination results in the synergistic reduction in tumor burden, tumor regression, and/or tumor development of said cancer, wherein said cancer is selected from the group consisting of: myeloma, multiple myeloma, smoldering myeloma, wherein said anti-CS1 antibody is elotuzumab, wherein said CD137 antibody is urelumab, wherein anti-CS1 antibody is administered at a dosage of about 1 to 10 mg/kg once every three weeks.

The present invention provides a method for treating a patient with cancer comprising the concurrent administration of a combination therapeutic regiment comprising: (i) a therapeutically effective amount of an agonistic CD137 antibody; and (ii) a therapeutically effective amount of an anti-CS1 antibody, wherein said combination results in the synergistic reduction in tumor burden, tumor regression, and/or tumor development of said cancer, wherein said cancer is selected from the group consisting of: myeloma, multiple myeloma, smoldering myeloma, wherein said anti-CS1 antibody is elotuzumab, wherein said CD137 antibody is urelumab, wherein said agonistic CD137 antibody is administered at a dosage of about 0.03-1 mg/kg, or about 3 mg-8 mg, and said anti-CS1 antibody is administered at a dosage of about 1 to 10 mg/kg once every three weeks.

The present invention provides a method for treating a patient with cancer comprising the concurrent administration of a combination therapeutic regiment comprising: (i) a therapeutically effective amount of an agonistic CD137 antibody; and (ii) a therapeutically effective amount of an anti-CS1 antibody, wherein said combination results in the synergistic reduction in tumor burden, tumor regression, and/or tumor development of said cancer, wherein said cancer is selected from the group consisting of: myeloma, multiple myeloma, smoldering myeloma, wherein said anti-CS1 antibody is elotuzumab, wherein said CD137 antibody is urelumab, wherein said agonistic CD137 antibody is administered at a dosage of about 0.03-1 mg/kg, or about 3 mg-8 mg, and said anti-CS1 antibody is administered at a dosage of about 1 mg/kg once every three weeks.

The present invention provides a method for treating a patient with cancer comprising the concurrent administration of a combination therapeutic regiment comprising: (i) a therapeutically effective amount of an agonistic CD137 antibody; and (ii) a therapeutically effective amount of an anti-CS1 antibody, wherein said combination results in the synergistic reduction in tumor burden, tumor regression, and/or tumor development of said cancer, wherein said cancer is selected from the group consisting of: myeloma, multiple myeloma, smoldering myeloma, wherein said anti-CS1 antibody is elotuzumab, wherein said CD137 antibody is urelumab, wherein said agonistic CD137 antibody is administered at a dosage of about 0.03-1 mg/kg, or about 3 mg-8 mg, and said anti-CS1 antibody is administered at a dosage of about 10 mg/kg once every three weeks.

The present invention provides a method for treating a patient with multiple myeloma comprising the sequential administration of a combination therapeutic regiment comprising: (i) first administering a therapeutically effective amount of an anti-CS1 antibody; followed by (ii) administering a therapeutically effective amount of an agonistic CD137 antibody; wherein said combination results in the synergistic reduction in tumor burden, tumor regression, and/or tumor development of said cancer.

The present invention provides a method for treating a patient with cancer comprising the sequential administration of a combination therapeutic regiment comprising: (i) first administering a therapeutically effective amount of an anti-CS1 antibody; followed by (ii) administering a therapeutically effective amount of an agonistic CD137 antibody; wherein said combination results in the synergistic reduction in tumor burden, tumor regression, and/or tumor development of said cancer, wherein said cancer is selected from the group consisting of: myeloma, multiple myeloma, smoldering myeloma.

The present invention provides a method for treating a patient with multiple myeloma comprising the sequential administration of a combination therapeutic regiment comprising: (i) first administering a therapeutically effective amount of an anti-CS1 antibody; followed by (ii) administering a therapeutically effective amount of an agonistic CD137 antibody; wherein said combination results in the synergistic reduction in tumor burden, tumor regression, and/or tumor development of said cancer, and wherein said anti-CS1 antibody is elotuzumab.

The present invention provides a method for treating a patient with cancer comprising the sequential administration of a combination therapeutic regiment comprising: (i) first administering a therapeutically effective amount of an anti-CS1 antibody; followed by (ii) administering a therapeutically effective amount of an agonistic CD137 antibody; wherein said combination results in the synergistic reduction in tumor burden, tumor regression, and/or tumor development of said cancer, wherein said cancer is selected from the group consisting of: myeloma, multiple myeloma, smoldering myeloma, and wherein said anti-CS1 antibody is elotuzumab.

The present invention provides a method for treating a patient with multiple myeloma comprising the sequential administration of a combination therapeutic regiment comprising: (i) first administering a therapeutically effective amount of an anti-CS1 antibody; followed by (ii) administering a therapeutically effective amount of an agonistic CD137 antibody; wherein said combination results in the synergistic reduction in tumor burden, tumor regression, and/or tumor development of said cancer, and wherein said agonistic CD137 antibody is urelumab.

The present invention provides a method for treating a patient with cancer comprising the sequential administration of a combination therapeutic regiment comprising: (i) first administering a therapeutically effective amount of an anti-CS1 antibody; followed by (ii) administering a therapeutically effective amount of an agonistic CD137 antibody; wherein said combination results in the synergistic reduction in tumor burden, tumor regression, and/or tumor development of said cancer, wherein said cancer is selected from the group consisting of: myeloma, multiple myeloma, smoldering myeloma, and wherein said agonistic CD137 antibody is urelumab.

The present invention provides a method for treating a patient with cancer comprising the sequential administration of a combination therapeutic regiment comprising: (i) first administering a therapeutically effective amount of an anti-CS1 antibody; followed by (ii) administering a therapeutically effective amount of an agonistic CD137 antibody; wherein said combination results in the synergistic reduction in tumor burden, tumor regression, and/or tumor development of said cancer, wherein said cancer is selected from the group consisting of: myeloma, multiple myeloma, smoldering myeloma, wherein said agonistic CD137 antibody is urelumab, and wherein said anti-CS1 antibody is elotuzumab.

The present invention provides a method for treating a patient with multiple myeloma comprising the concurrent administration of a combination therapeutic regiment comprising: (i) first administering one or more cycles of a therapeutically effective amount of an anti-CS1 antibody; and followed by (ii) one or more cycles of a therapeutically effective amount of an agonistic CD137 antibody, wherein said combination results in the synergistic reduction in tumor burden, tumor regression, and/or tumor development of said cancer.

The present invention provides a method for treating a patient with multiple myeloma comprising the sequential administration of a combination therapeutic regiment comprising: (i) one or more cycles of a therapeutically effective amount of an agonistic CD137 antibody; and (ii) one or more cycles of a therapeutically effective amount of an anti-CS1 antibody, wherein said combination results in the synergistic reduction in tumor burden, tumor regression, and/or tumor development of said cancer.

The present invention provides a method for treating a patient with cancer comprising the sequential administration of a combination therapeutic regiment comprising: (i) first administering a therapeutically effective amount of an anti-CS1 antibody; followed by (ii) administering a therapeutically effective amount of an agonistic CD137 antibody; wherein said combination results in the synergistic reduction in tumor burden, tumor regression, and/or tumor development of said cancer, wherein said cancer is selected from the group consisting of: myeloma, multiple myeloma, smoldering myeloma, wherein said agonistic CD137 antibody is urelumab, wherein said anti-CS1 antibody is elotuzumab, and wherein said agonistic CD137 antibody is administered at a dosage of about 0.03-1 mg/kg, or about 3 mg-8 mg, and said anti-CS1 antibody is administered at a dosage of about 10 mg/kg once every three weeks.

The present invention provides a method for treating a patient with cancer with a sequential administration of a combination therapeutic regiment comprising: (i) first administering a therapeutically effective amount of an anti-CS1 antibody; followed by (ii) administering a therapeutically effective amount of an agonistic CD137 antibody; wherein said method optionally comprises an Intervening Period in-between (i) and (ii), wherein said Intervening Period is between 0 days to 24 weeks in time. In one aspect of the present invention, the Intervening Period is between 2 to 8 weeks. In one aspect of the present invention, the Intervening Period is between 3 to 6 weeks. In one aspect of the present invention, the Intervening Period is between 1 to 2 weeks. In one aspect of the present invention, the Intervening Period is between 3 to 7 days. In one aspect of the present invention, the Intervening Period is between about 1 to 3 days. In one aspect of the present invention, the Intervening Period is about 2 days. In one aspect of the present invention, the Intervening Period is about 1 day.

In another aspect, methods of treating multiple myeloma in a human patient are provided, the methods comprising administering to the patient, an effective amount of each of:

(a) an agonistic CD137 antibody comprising the CDR1, CDR2 and CDR3 domains in a heavy chain variable region comprising the sequence set forth in SEQ ID NO:4, and the CDR1, CDR2 and CDR3 domains in a light chain variable region comprising the sequence set forth in SEQ ID NO:3, and

(b) an anti-CS1 antibody comprising the CDR1, CDR2 and CDR3 domains in a heavy chain variable region comprising the sequence set forth in SEQ ID NO:2, and the CDR1, CDR2 and CDR3 domains in a light chain variable region comprising the sequence set forth in SEQ ID NO:1,

wherein the anti-CS1 antibody is administered weekly for a total of 8 doses over 8 weeks and the agonistic CD137 antibody is administered every 3 weeks for a total of 3 doses over 8 weeks during an induction phase, and

wherein the agonistic CD137 antibody is administered at a dose of 0.03-1 mg/kg body weight, or about 3 mg-8 mg body weight and the anti-CS1 antibody is administered at a dose of 0.1-20 mg/kg body weight during both the induction and maintenance phases.

In another aspect, methods of treating multiple myeloma in a human patient are provided, the methods comprising administering to the patient, an effective amount of each of:

(a) an agonistic CD137 antibody comprising the CDR1, CDR2 and CDR3 domains in a heavy chain variable region comprising the sequence set forth in SEQ ID NO:4, and the CDR1, CDR2 and CDR3 domains in a light chain variable region comprising the sequence set forth in SEQ ID NO:3, and

(b) an anti-CS1 antibody comprising the CDR1, CDR2 and CDR3 domains in a heavy chain variable region comprising the sequence set forth in SEQ ID NO:2, and the CDR1, CDR2 and CDR3 domains in a light chain variable region comprising the sequence set forth in SEQ ID NO:1,

wherein the anti-CS1 antibody is administered weekly for a total of 8 doses over 8 weeks and the agonistic CD137 antibody is administered every 3 weeks for a total of 3 doses over 8 weeks during an induction phase, and

wherein the agonistic CD137 antibody is administered at a dose of 0.03 mg/kg body weight and the anti-CS1 antibody is administered at a dose of 0.1-20 mg/kg body weight during both the induction and maintenance phases.

In another aspect, methods of treating multiple myeloma in a human patient are provided, the methods comprising administering to the patient, an effective amount of each of:

(a) an agonistic CD137 antibody comprising the CDR1, CDR2 and CDR3 domains in a heavy chain variable region comprising the sequence set forth in SEQ ID NO:4, and the CDR1, CDR2 and CDR3 domains in a light chain variable region comprising the sequence set forth in SEQ ID NO:3, and

(b) an anti-CS1 antibody comprising the CDR1, CDR2 and CDR3 domains in a heavy chain variable region comprising the sequence set forth in SEQ ID NO:2, and the CDR1, CDR2 and CDR3 domains in a light chain variable region comprising the sequence set forth in SEQ ID NO:1,

wherein the anti-CS1 antibody is administered weekly for a total of 8 doses over 8 weeks and the agonistic CD137 antibody is administered every 3 weeks for a total of 3 doses over 8 weeks during an induction phase, and

wherein the agonistic CD137 antibody is administered at a dose of 0.1 mg/kg body weight and the anti-CS1 antibody is administered at a dose of 0.1-20 mg/kg body weight during both the induction and maintenance phases.

In another aspect, methods of treating multiple myeloma in a human patient are provided, the methods comprising administering to the patient, an effective amount of each of:

(a) an agonistic CD137 antibody comprising the CDR1, CDR2 and CDR3 domains in a heavy chain variable region comprising the sequence set forth in SEQ ID NO:4, and the CDR1, CDR2 and CDR3 domains in a light chain variable region comprising the sequence set forth in SEQ ID NO:3, and

(b) an anti-CS1 antibody comprising the CDR1, CDR2 and CDR3 domains in a heavy chain variable region comprising the sequence set forth in SEQ ID NO:2, and the CDR1, CDR2 and CDR3 domains in a light chain variable region comprising the sequence set forth in SEQ ID NO:1,

wherein the anti-CS1 antibody is administered weekly for a total of 8 doses over 8 weeks and the agonistic CD137 antibody is administered every 3 weeks for a total of 3 doses over 8 weeks during an induction phase, and

wherein the agonistic CD137 antibody is administered at a dose of 0.3 mg/kg body weight and the anti-CS1 antibody is administered at a dose of 0.1-20 mg/kg body weight during both the induction and maintenance phases.

In another aspect, methods of treating multiple myeloma in a human patient are provided, the methods comprising administering to the patient, an effective amount of each of:

(a) an agonistic CD137 antibody comprising the CDR1, CDR2 and CDR3 domains in a heavy chain variable region comprising the sequence set forth in SEQ ID NO:4, and the CDR1, CDR2 and CDR3 domains in a light chain variable region comprising the sequence set forth in SEQ ID NO:3, and

(b) an anti-CS1 antibody comprising the CDR1, CDR2 and CDR3 domains in a heavy chain variable region comprising the sequence set forth in SEQ ID NO:2, and the CDR1, CDR2 and CDR3 domains in a light chain variable region comprising the sequence set forth in SEQ ID NO:1,

wherein (A) the anti-CS1 antibody is administered weekly for a total of 8 doses over 8 weeks and the agonistic CD137 antibody is administered every 3 weeks for a total of 3 doses over 8 weeks during an induction phase, followed by (B) administration of the anti-CS1 antibody every 2 weeks and administration of the agonistic CD137 antibody every 4 weeks during a maintenance phase, and

wherein the agonistic CD137 antibody is administered at a dose of 0.1-20 mg/kg body weight and the anti-CS1 antibody is administered at a dose of 0.1-20 mg/kg body weight during both the induction and maintenance phases.

In another aspect, methods of treating multiple myeloma in a human patient are provided, the methods comprising administering to the patient, an effective amount of each of:

(a) an agonistic CD137 antibody comprising the CDR1, CDR2 and CDR3 domains in a heavy chain variable region comprising the sequence set forth in SEQ ID NO:4, and the CDR1, CDR2 and CDR3 domains in a light chain variable region comprising the sequence set forth in SEQ ID NO:3, and

(b) an anti-CS1 antibody comprising the CDR1, CDR2 and CDR3 domains in a heavy chain variable region comprising the sequence set forth in SEQ ID NO:2, and the CDR1, CDR2 and CDR3 domains in a light chain variable region comprising the sequence set forth in SEQ ID NO:1,

wherein (A) the anti-CS1 antibody is administered weekly for a total of 8 doses over 8 weeks and the agonistic CD137 antibody is administered every 3 weeks for a total of 3 doses over 8 weeks during an induction phase, followed by (B) administration of the anti-CS1 antibody every 2 weeks and administration of the agonistic CD137 antibody every 4 weeks during a maintenance phase, and

wherein the agonistic CD137 antibody is administered at a dose of 0.03 mg/kg body weight or 3 mg, and the anti-CS1 antibody is administered at a dose of 0.1-20 mg/kg body weight during both the induction and maintenance phases.

In another aspect, methods of treating multiple myeloma in a human patient are provided, the methods comprising administering to the patient, an effective amount of each of:

(a) an agonistic CD137 antibody comprising the CDR1, CDR2 and CDR3 domains in a heavy chain variable region comprising the sequence set forth in SEQ ID NO:4, and the CDR1, CDR2 and CDR3 domains in a light chain variable region comprising the sequence set forth in SEQ ID NO:3, and

(b) an anti-CS1 antibody comprising the CDR1, CDR2 and CDR3 domains in a heavy chain variable region comprising the sequence set forth in SEQ ID NO:2, and the CDR1, CDR2 and CDR3 domains in a light chain variable region comprising the sequence set forth in SEQ ID NO:1,

wherein (A) the anti-CS1 antibody is administered weekly for a total of 8 doses over 8 weeks and the agonistic CD137 antibody is administered every 3 weeks for a total of 3 doses over 8 weeks during an induction phase, followed by (B) administration of the anti-CS1 antibody every 2 weeks and administration of the agonistic CD137 antibody every 4 weeks during a maintenance phase, and

wherein the agonistic CD137 antibody is administered at a dose of 0.1 mg/kg body weight or 8 mg, and the anti-CS1 antibody is administered at a dose of 0.1-20 mg/kg body weight during both the induction and maintenance phases.

In another aspect, methods of treating multiple myeloma in a human patient are provided, the methods comprising administering to the patient, an effective amount of each of:

(a) an agonistic CD137 antibody comprising the CDR1, CDR2 and CDR3 domains in a heavy chain variable region comprising the sequence set forth in SEQ ID NO:4, and the CDR1, CDR2 and CDR3 domains in a light chain variable region comprising the sequence set forth in SEQ ID NO:3, and

(b) an anti-CS1 antibody comprising the CDR1, CDR2 and CDR3 domains in a heavy chain variable region comprising the sequence set forth in SEQ ID NO:2, and the CDR1, CDR2 and CDR3 domains in a light chain variable region comprising the sequence set forth in SEQ ID NO:1,

wherein (A) the anti-CS1 antibody is administered weekly for a total of 8 doses over 8 weeks and the agonistic CD137 antibody is administered every 3 weeks for a total of 3 doses over 8 weeks during an induction phase, followed by (B) administration of the anti-CS1 antibody every 2 weeks and administration of the agonistic CD137 antibody every 4 weeks during a maintenance phase, and

wherein the agonistic CD137 antibody is administered at a dose of 0.1 mg/kg body weight and the anti-CS1 antibody is administered at a dose of 0.1-20 mg/kg body weight during both the induction and maintenance phases.

In another aspect, methods of treating multiple myeloma in a human patient are provided, the methods comprising administering to the patient, an effective amount of each of:

(a) an agonistic CD137 antibody comprising the CDR1, CDR2 and CDR3 domains in a heavy chain variable region comprising the sequence set forth in SEQ ID NO:4, and the CDR1, CDR2 and CDR3 domains in a light chain variable region comprising the sequence set forth in SEQ ID NO:3, and

(b) an anti-CS1 antibody comprising the CDR1, CDR2 and CDR3 domains in a heavy chain variable region comprising the sequence set forth in SEQ ID NO:2, and the CDR1, CDR2 and CDR3 domains in a light chain variable region comprising the sequence set forth in SEQ ID NO:1,

wherein (A) the anti-CS1 antibody is administered weekly for a total of 8 doses over 8 weeks and the agonistic CD137 antibody is administered every 3 weeks for a total of 3 doses over 8 weeks during an induction phase, followed by (B) administration of the anti-CS1 antibody every 2 weeks and administration of the agonistic CD137 antibody every 4 weeks during a maintenance phase, and

wherein the agonistic CD137 antibody is administered at a dose of 0.3 mg/kg body weight and the anti-CS1 antibody is administered at a dose of 0.1-20 mg/kg body weight during both the induction and maintenance phases.

In certain embodiments, each dose of the agonistic CD137 antibody is administered at about 0.3, 0.1, 0.3, 1, 3, 6, 10 or 20 mg/kg. In preferred embodiments, each dose of the agonistic CD137 antibody is administered at 0.03 mg/kg, 0.1 mg/kg, 1 mg/kg or 3 mg/kg; or 3 mg or 8 mg. In other embodiments, each dose of the anti-CS1 antibody is administered at 0.1, 0.3, 1, 3, 6, 10 or 20 mg/kg body weight. In a preferred embodiment, each dose of the anti-CS1 antibody is administered at 10 mg/kg.

In one embodiment, the agonistic CD137 antibody and anti-CS1 antibody are administered at the following doses during either the induction or maintenance phase:

(a) 0.03 mg/kg agonistic CD137 antibody and 10 mg/kg of anti-CS1 antibody;

(b) 0.1 mg/kg agonistic CD137 antibody and 10 mg/kg of anti-CS1 antibody;

(c) 0.3 mg/kg agonistic CD137 antibody and 10 mg/kg of anti-CS1 antibody;

(d) 1 mg/kg agonistic CD137 antibody and 10 mg/kg of anti-CS1 antibody; or

(e) 3 mg/kg agonistic CD137 antibody and 10 mg/kg of anti-CS1 antibody.

In one embodiment, the agonistic CD137 antibody and anti-CS1 antibody are administered at the following doses during either the induction or maintenance phase:

(a) 0.03 mg/kg agonistic CD137 antibody and 1 mg/kg of anti-CS1 antibody;

(b) 0.1 mg/kg agonistic CD137 antibody and 1 mg/kg of anti-CS1 antibody;

(c) 0.3 mg/kg agonistic CD137 antibody and 1 mg/kg of anti-CS1 antibody;

(d) 1 mg/kg agonistic CD137 antibody and 1 mg/kg of anti-CS1 antibody; or

(e) 3 mg/kg agonistic CD137 antibody and 1 mg/kg of anti-CS1 antibody.

In certain embodiments, each dose of the agonistic CD137 antibody is administered at about 3 mg, 4 mg, 5 mg, 6 mg, 7 mg, 8 mg, 9 mg, 10 mg, 11 mg, 12 mg, 13 mg, 14 mg, 15 mg, 16 mg, 17 mg, 18 mg, 19 mg, or 20 mg. In preferred embodiments, each dose of the agonistic CD137 antibody is administered at about 3 mg or 8 mg. In other embodiments, each dose of the anti-CS1 antibody is administered at 0.1, 0.3, 1, 3, 6, 10 or 20 mg/kg body weight. In a preferred embodiment, each dose of the anti-CS1 antibody is administered at 10 mg/kg.

In one embodiment, the agonistic CD137 antibody and anti-CS1 antibody are administered at the following doses during either the induction or maintenance phase:

(a) 3 mg agonistic CD137 antibody and 10 mg/kg of anti-CS1 antibody; or

(b) 8 mg agonistic CD137 antibody and 10 mg/kg of anti-CS1 antibody.

In one embodiment, the agonistic CD137 antibody and anti-CS1 antibody are administered at the following doses during either the induction or maintenance phase:

(a) 3 mg agonistic CD137 antibody and 10 mg/kg of anti-CS1 antibody; or

(b) 8 mg agonistic CD137 antibody and 10 mg/kg of anti-CS1 antibody.

In one embodiment, the anti-CS1 antibody is administered on (1) day 1, week 1, (2) day 1, week 2, (3), day 1, week 3, (4), day 1, week 4, (5) day 1, week 5, (6) day 1, week 6, (7) day 1, week 7, and (8) day 1, week 8, of the induction phase. In another embodiment, the agonistic CD137 antibody is administered on (1) day 1, week 1, (2) day 1, week 4, and (3) day 1, week 7 of the induction phase. In another embodiment, the anti-CS1 antibody is administered on (1) day 1, week 10 and (2) day 1, week 15 of the maintenance phase. In another embodiment, the agonistic CD137 antibody is administered on (1) day 1, week 10 of the maintenance phase. In another embodiment, the maintenance phase is repeated for at least 1, 2, 3, 4, 5, 6, 7, 8, 9, 10, 11, 12, 13, 14, 15, 16, 17, 18, 19, or 20 or more cycles.

In one embodiment, the anti-CS1 antibody and agonistic CD137 antibody are administered as a first (“front”) line of treatment (e.g., the initial or first treatment). In another embodiment, the anti-CS1 antibody and agonistic CD137 antibody are administered as a second line of treatment (e.g., after initial treatment with the same or a different therapeutic, including after relapse and/or where the first treatment has failed).

The agonistic CD137 antibody and anti-CS1 antibodies can be administered to a subject by any suitable means. In one embodiment, the antibodies are formulated for intravenous administration. In another embodiment, the antibodies are administered simultaneously (e.g., in a single formulation or concurrently as separate formulations). Alternatively, in another embodiment, the antibodies are administered sequentially (e.g., as separate formulations).

The efficacy of the treatment methods provided herein can be assessed using any suitable means. In one embodiment, the treatment produces at least one therapeutic effect selected from the group consisting of complete response, very good partial response, partial response, and stable disease. In another embodiment, administration of an agonistic CD137 antibody and an anti-CS1 antibody has a synergistic effect on treatment compared to administration of either antibody alone.

Also provided are compositions comprising:

(a) an agonistic CD137 antibody comprising the CDR1, CDR2 and CDR3 domains in a heavy chain variable region comprising the sequence set forth in SEQ ID NO:4, and the CDR1, CDR2 and CDR3 domains in a light chain variable region comprising the sequence set forth in SEQ ID NO:3, and

(b) an anti-CS1 antibody comprising the CDR1, CDR2 and CDR3 domains in a heavy chain variable region comprising the sequence set forth in SEQ ID NO:2, and the CDR1, CDR2 and CDR3 domains in a light chain variable region comprising the sequence set forth in SEQ ID NO:1.

The invention further provides kits that include a pharmaceutical composition containing an agonistic CD137, such as urelumab, and an anti-CS1 antibody, such as elotuzumab, and a pharmaceutically-acceptable carrier, in a therapeutically effective amount adapted for use in the methods described herein. In one embodiment, the kit comprises:

(a) a dose of an agonistic CD137 antibody comprising the CDR1, CDR2 and CDR3 domains in a heavy chain variable region comprising the sequence set forth in SEQ ID NO:4, and the CDR1, CDR2 and CDR3 domains in a light chain variable region comprising the sequence set forth in SEQ ID NO:3, and

(b) a dose of an anti-CS1 antibody comprising the CDR1, CDR2 and CDR3 domains in a heavy chain variable region comprising the sequence set forth in SEQ ID NO:2, and the CDR1, CDR2 and CDR3 domains in a light chain variable region comprising the sequence set forth in SEQ ID NO:1; and

(c) instructions for using the agonistic CD137 antibody and anti-CS1 antibody in a method of the in the invention.

In another aspect, an agonistic CD137 antibody is provided, the agonistic CD137 antibody comprising the CDR1, CDR2 and CDR3 domains in a heavy chain variable region comprising the sequence set forth in SEQ ID NO:4, and the CDR1, CDR2 and CDR3 domains in a light chain variable region comprising the sequence set forth in SEQ ID NO:3, for co-administration with an anti-CS1 antibody comprising the CDR1, CDR2 and CDR3 domains in a heavy chain variable region comprising the sequence set forth in SEQ ID NO:2, and the CDR1, CDR2 and CDR3 domains in a light chain variable region comprising the sequence set forth in SEQ ID NO:1.

In a further aspect, an agonistic CD137 antibody is provided, the agonistic CD137 antibody comprising the CDR1, CDR2 and CDR3 domains in a heavy chain variable region comprising the sequence set forth in SEQ ID NO:4, and the CDR1, CDR2 and CDR3 domains in a light chain variable region comprising the sequence set forth in SEQ ID NO:3, for co-administration with an anti-CS1 antibody comprising the CDR1, CDR2 and CDR3 domains in a heavy chain variable region comprising the sequence set forth in SEQ ID NO:2, and the CDR1, CDR2 and CDR3 domains in a light chain variable region comprising the sequence set forth in SEQ ID NO:1,

wherein (A) the anti-CS1 antibody is administered weekly for a total of 8 doses over 8 weeks and the agonistic CD137 antibody is administered every 3 weeks for a total of 3 doses over 8 weeks during an induction phase, followed by (B) administration of the anti-CS1 antibody every 2 weeks and administration of the agonistic CD137 antibody every 4 weeks during a maintenance phase, and

wherein the agonistic CD137 antibody is administered at a dose of 0.1-20 mg/kg body weight and the anti-CS1 antibody is administered at a dose of 0.1-20 mg/kg body weight during both the induction and maintenance phases.

In a further aspect, an agonistic CD137 antibody is provided, the agonistic CD137 antibody comprising the CDR1, CDR2 and CDR3 domains in a heavy chain variable region comprising the sequence set forth in SEQ ID NO:4, and the CDR1, CDR2 and CDR3 domains in a light chain variable region comprising the sequence set forth in SEQ ID NO:3, for co-administration with an anti-CS1 antibody comprising the CDR1, CDR2 and CDR3 domains in a heavy chain variable region comprising the sequence set forth in SEQ ID NO:2, and the CDR1, CDR2 and CDR3 domains in a light chain variable region comprising the sequence set forth in SEQ ID NO:1,

wherein (A) the anti-CS1 antibody is administered weekly for a total of 8 doses over 8 weeks and the agonistic CD137 antibody is administered every 3 weeks for a total of 3 doses over 8 weeks during an induction phase, followed by (B) administration of the anti-CS1 antibody every 2 weeks and administration of the agonistic CD137 antibody every 4 weeks during a maintenance phase, and

wherein the agonistic CD137 antibody is administered at a dose of 0.03-0.1 mg/kg body weight and the anti-CS1 antibody is administered at a dose of 0.1-20 mg/kg body weight during both the induction and maintenance phases.

In a further aspect, an agonistic CD137 antibody is provided, the agonistic CD137 antibody comprising the CDR1, CDR2 and CDR3 domains in a heavy chain variable region comprising the sequence set forth in SEQ ID NO:4, and the CDR1, CDR2 and CDR3 domains in a light chain variable region comprising the sequence set forth in SEQ ID NO:3, for co-administration with an anti-CS1 antibody comprising the CDR1, CDR2 and CDR3 domains in a heavy chain variable region comprising the sequence set forth in SEQ ID NO:2, and the CDR1, CDR2 and CDR3 domains in a light chain variable region comprising the sequence set forth in SEQ ID NO:1,

wherein (A) the anti-CS1 antibody is administered weekly for a total of 8 doses over 8 weeks and the agonistic CD137 antibody is administered every 3 weeks for a total of 3 doses over 8 weeks during an induction phase, followed by (B) administration of the anti-CS1 antibody every 2 weeks and administration of the agonistic CD137 antibody every 4 weeks during a maintenance phase, and

wherein the agonistic CD137 antibody is administered at a dose of between 3 mg-8 mg and the anti-CS1 antibody is administered at a dose of 0.1-20 mg/kg body weight during both the induction and maintenance phases.

The invention further provides kits that include a pharmaceutical composition containing an agonistic CD137, such as urelumab, and an anti-CS1 antibody, such as elotuzumab, and a pharmaceutically-acceptable carrier, in a therapeutically effective amount adapted for use in the methods described herein. In one embodiment, the kit comprises:

(a) a dose of an agonistic CD137 antibody comprising the CDR1, CDR2 and CDR3 domains in a heavy chain variable region comprising the sequence set forth in SEQ ID NO:4, and the CDR1, CDR2 and CDR3 domains in a light chain variable region comprising the sequence set forth in SEQ ID NO:3, and

(b) a dose of an anti-CS1 antibody comprising the CDR1, CDR2 and CDR3 domains in a heavy chain variable region comprising the sequence set forth in SEQ ID NO:2, and the CDR1, CDR2 and CDR3 domains in a light chain variable region comprising the sequence set forth in SEQ ID NO:1, and

(c) instructions for first administering the anti-CS1 antibody followed by the agonistic CD137 antibody thereafter.

In another aspect, an agonistic CD137 antibody is provided, the agonistic CD137 antibody comprising the CDR1, CDR2 and CDR3 domains in a heavy chain variable region comprising the sequence set forth in SEQ ID NO:4, and the CDR1, CDR2 and CDR3 domains in a light chain variable region comprising the sequence set forth in SEQ ID NO:3, for sequential administration with an anti-CS1 antibody comprising the CDR1, CDR2 and CDR3 domains in a heavy chain variable region comprising the sequence set forth in SEQ ID NO:2, and the CDR1, CDR2 and CDR3 domains in a light chain variable region comprising the sequence set forth in SEQ ID NO:1, wherein the anti-CS1 antibody is administered first followed by the agonistic CD137 antibody.

### BRIEF DESCRIPTION OF THE FIGURES/DRAWINGS

FIGS. 1A-B. Antitumor Activity of Elotuzumab, CD137 antibody or their combination in the OPM-2 Tumor model following different schedules of administration. Elotuzumab was administered at either 20 (A); or 100 (B) μg/mouse on Day 8. CD137 mAb was administered at 100 μg/mouse starting on the same day, or one day before, or one day after elotuzumab administration. As shown in FIG. 1A, the combination of Elotuzumab and the CD137 antibody resulted in synergistic inhibition of tumor growth in the OPM-2 tumor model, relative to results obtained from administration of either Elotuzumab or the CD137 antibody alone. However, as shown in FIG. 1B, the combination of Elotuzumab and the CD137 antibody resulted in synergistic inhibition of tumor growth in the OPM-2 tumor model only when both were administered concurrently, but not when administered sequentially, when lower doses of Elotuzumab were used.

FIGS. 2A-F. Antitumor Activity of Elotuzumab, CD137 antibody or their combination in the OPM-2 Tumor model following different schedules of administration. Mice were administered one of the following regimens: (A) Control vehicle; (B) CD137 mAb, 100 μg/mouse; (C) Elotuzumab 100 μg/mouse; (D) Elotuzumab (100) Day 8+CD137 (100) Days 8, 15, 22; (E) Elotuzumab (100) Day 8+CD137 (100) Days 9, 16, 23; (F) CD137 (100) Days 8, 15, 22+Elotuzumab Day 9. As shown, the combination of Elotuzumab and the CD137 antibody resulted in consistent synergistic inhibition of tumor growth in the OPM-2 tumor model only when both were administered concurrently, but less consistently when administered sequentially.

FIGS. 3A-D. Effect of concurrent administration of CD137 mAb and Elotuzumab at 1 μg/mouse in the OPM-2 multiple myeloma tumor model. Mice were administered one of the following regimens: (A) Control vehicle; (B) CD137 mAb, 100 μg/mouse; (C) Elotuzumab 1 μg/mouse; (D) Elotuzumab 1 μg/mouse+CD137 mAb 100 μg/mouse. As shown, tumor growth was only modestly inhibited when lower doses of Elotuzumab were administered.

FIGS. 4A-D. Effect of concurrent administration of CD137 mAb and Elotuzumab at 10 μg/mouse in the OPM-2 multiple myeloma tumor model. Mice were administered one of the following regimens: (A) Control vehicle; (B) CD137 mAb, 100 μg/mouse; (C) Elotuzumab 10 μg/mouse; (D) Elotuzumab 10 μg/mouse+CD137 mAb 100 μg/mouse. As shown, tumor growth was significantly, and synergistically inhibited when the higher dose of Elotuzumab (10 μg) was administered in combination with the CD137 mAb.

FIGS. 5A-D. Effect of concurrent administration of CD137 mAb and Elotuzumab at 100 μg/mouse in the OPM-2 multiple myeloma tumor model. Mice were administered one of the following regimens: (A) Control vehicle; (B) CD137 mAb, 100 μg/mouse; (C) Elotuzumab 100 μg/mouse; (D) Elotuzumab 100 μg/mouse+CD137 mAb 100 μg/mouse. As shown, tumor growth was completely inhibited at synergistic levels, when the highest dose of Elotuzumab (100 μg) was administered in combination with the CD137 mAb.

FIGS. 6A-D. Effect of concurrent administration of CD137 mAb at 1 μg/mouse and Elotuzumab at 100 μg/mouse in the OPM-2 multiple myeloma tumor model. Mice were administered one of the following regimens: (A) Control vehicle; (B) Elotuzumab 100 μg/mouse; (C) CD137 mAb, 1 μg/mouse; (D) Elotuzumab 100 μg/mouse+CD137 mAb 1 μg/mouse. As shown, tumor growth was consistently inhibited when Elotuzumab (100 μg) was administered in combination with the CD137 mAb at 1 μg/mouse relative to either agent alone.

FIGS. 7A-D. Effect of concurrent administration of CD137 mAb at 10 μg/mouse and Elotuzumab at 100 μg/mouse in the OPM-2 multiple myeloma tumor model. Mice were administered one of the following regimens: (A) Control vehicle; (B) Elotuzumab 100 μg/mouse; (C) CD137 mAb, 10 μg/mouse; (D) Elotuzumab 100 μg/mouse+CD137 mAb 10 μg/mouse. As shown, tumor growth was significantly inhibited when Elotuzumab (100 μg) was administered in combination with the CD137 mAb at 10 μg/mouse relative to either agent alone.

FIGS. 8A-D. Effect of concurrent administration of CD137 mAb at 100 μg/mouse and Elotuzumab at 100 μg/mouse in the OPM-2 multiple myeloma tumor model. Mice were administered one of the following regimens: (A) Control vehicle; (B) Elotuzumab 100 μg/mouse; (C) CD137 mAb, 100 μg/mouse; (D) Elotuzumab 100 μg/mouse+CD137 mAb 100 μg/mouse. As shown, tumor growth was significantly inhibited when Elotuzumab (100 μg) was administered in combination with the CD137 mAb at 100 μg/mouse relative to either agent alone.

FIG. 9 is a schematic depicting a study design for a phase I trial. Elotuzumab is depicted as an “E”, while urelumab is depicted as a “U”.

### DETAILED DESCRIPTION OF THE INVENTION

The present invention is based on data from preclinical studies conducted in female SCID mice (6-8 weeks old) that were implanted SC (subcutaneous implantation) with the multiple myeloma cell line OPM-2 which were treated with Elotuzumab IP (intraperitoneal administration) alone, or treated with CD137 mAb (BMS-469492—a monoclonal antibody directed against mouse CD137) alone or concurrently or sequentially in combination with each other. The results demonstrated for the first time that the combination of elotuzumab and CD137 mAb resulted in higher number of mice exhibiting complete tumor responses compared to elotuzumab or CD137 mAb alone. In particular, independent of the dose of elotuzumab administered (20 μg or 100 μg), when CD137 mAb and elotuzumab were administered on the same day, complete regressions were observed in ≧50% mice (4 out of 8 mice, and 6 out of 8 mice with the combination of CD137 mAb (100 μg/mouse) plus elotuzumab at 20 μg/mouse or 100 μg/mouse respectively). In addition, greater numbers of mice with complete regressions were observed in the combination therapy groups with elotuzumab administered at the highest dose (100 μg/mouse) following any of the schedules tested. Based on these results, concurrent dosing of both therapeutic agents was selected for further studies exploring various dose levels.

The present invention is also based on data from preclinical studies designed to evaluate the efficacy of concurrent administration of CD137 mAb (100 μg/mouse) in combination with elotuzumab administered at various dose levels (1, 10, 100 μg/mouse) in the OPM-2 multiple myeloma tumor model. Elotuzumab as single agent demonstrated a dose-dependent effect with enhanced antitumor activity at 100 μg/mouse, while CD137 agonist antibody did not elicit significant antitumor activity. Consistent with the prior experiments disclosed herein, combination therapy demonstrated greater activity with higher dose levels of elotuzumab. Marked increases in the number of mice with complete regressions were observed in the experimental groups that received CD137 mAb plus elotuzumab at 10 and 100 μg/mouse compared to elotuzumab or CD137 mAb alone.

The present invention is also based on data from preclinical studies designed to evaluate the effect of combination therapy of elotuzumab (100 μg/mouse) with CD137 mAb at 1, 10, and 100 μg/mouse. No significant antitumor effect was observed with CD137 mAb alone at any dose level; elotuzumab at 100 μg/mouse alone demonstrated a delay in tumor growth but no tumor regressions (70% TGI). Conversely, a greater number of mice with complete regressions was observed in the combination groups treated with CD137 mAb at 10 and 100 μg/mouse plus elotuzumab compared to elotuzumab or CD137 mAb alone.

The teachings of the present invention are believed to be the first association between the concurrent administration of an anti-CS1 agent in combination with an agonist CD137 agent with increased, and in some cases synergistic, outcomes in terms of efficacy, safety, and tolerability.

The teachings of the present invention are believed to be the first association between the concurrent administration of an anti-CS1 agent in combination with an agonist CD137 agent with increased, and in some cases synergistic, outcomes in terms of efficacy, safety, and tolerability, particularly when the anti-CS1 agent is administered at a dose between about 1-10 mg/kg, and the agonist CD137 agent is administered at a dose between about 0.1-1 mg/kg, in other embodiments, administered at a dose between about 0.03 mg/kg-0.1 mg/kg, and in other embodiments, administered at a dose between about 3 mg-8 mg.

The teachings of the present invention are believed to be the first association between the sequential administration of an anti-CS1 agent in combination with an agonist CD137 agent with increased, and in some cases synergistic, outcomes in terms of efficacy, safety, and tolerability.

The teachings of the present invention are believed to be the first association between the sequential administration of an anti-CS1 agent in combination with an agonist CD137 agent with increased, and in some cases synergistic, outcomes in terms of efficacy, safety, and tolerability, particularly when the anti-CS-1 agent is administered first followed by an agonist CD137 agent.

In one aspect of the present invention, the sequential administration of one or more cycles of an anti-CS1 agent followed by one or more cycles comprising an agonist CD137 agent, may optionally comprise an “Intervening Period”, defined as a time period beginning from the end of the last anti-CS1 agent cycle up until the beginning of the agonist CD137 agent cycle. In another aspect of the present invention, the sequential administration of one or more cycles of an agonist CD137 agent followed by one or more cycles comprising an anti-CS1 agent, may optionally comprise an “Intervening Period”, defined as a time period beginning from the end of the last anti-CS1 agent cycle up until the beginning of the agonist CD137 agent cycle. The Intervening Period may be about 24 weeks. In another embodiment of the present invention, the Intervening Period may be about 20 weeks. In another embodiment of the present invention, the Intervening Period may be about 18 weeks. In another embodiment of the present invention, the Intervening Period may be about 15 weeks. In another embodiment of the present invention, the Intervening Period may be about 12 weeks. In another embodiment of the present invention, the Intervening Period may be about 11 weeks. In another embodiment of the present invention, the Intervening Period may be about 10 weeks. In another embodiment of the present invention, the Intervening Period may be about 9 weeks. In another embodiment of the present invention, the Intervening Period may be about 8 weeks. In another embodiment of the present invention, the Intervening Period may be about 7 weeks. In another embodiment of the present invention, the Intervening Period may be about 6 weeks. In another embodiment of the present invention, the Intervening Period may be about 5 weeks. In another embodiment of the present invention, the Intervening Period may be about 4 weeks. In another embodiment of the present invention, the Intervening Period may be about 3 weeks. In another embodiment of the present invention, the Intervening Period may be about 2 weeks. In another embodiment of the present invention, the Intervening Period may be about 1 week. In another embodiment of the present invention, the Intervening Period may be about 1, 2, 3, 4, 5, 6, or 7 days. In this context, the term “about” shall be construed to mean±1, 2, 3, 4, 5, 6, or 7 days more or less than the stated Intervening Period.

In one embodiment of the present invention, the Intervening Period is between 2 to 8 weeks. In another embodiment of the present invention, the Intervening Period is between 3 to 6 weeks.

In one embodiment of the present invention, the Intervening Period is one day.

In another embodiment of the present invention, the Intervening Period may be less than 0 days such that the anti-CS1 agent is administered concurrently with the agonist CD137 agent.

The phrase “an agonist CD137 cycle” or “cycle of an agonist CD137 agent” or is meant to encompass either one or more dosing cycle(s) of an agonist CD137 agent, or one or more dosing cycle(s) of a combination comprising one or more agonist CD137 agent(s).

The phrase “an anti-CS1 cycle” or “cycle of an anti-CS1 agent” or “cycles of a therapeutically effective amount of an anti-CS1 antibody” is meant to encompass either one or more dosing cycle(s) of an anti-CS1 agent, or one or more dosing cycle(s) of a combination comprising one or more anti-CS1 agent(s).

For the purposes of the present invention, “one or more cycles of an agonist CD137 cycle” and/or “one or more cycles of an agonist CD137 agent” means at least 1, at least 2, at least 3, at least 4, at least 5, at least 6, at least 7, at least 8, at least 9, or at least 10 cycles of primary treatment with either agent(s), followed by one or more optional maintenance cycles of either agent(s). The maintenance cycle(s) may follow a similar number of cycles as outlined for the primary therapy, or may be significantly longer or shorter in terms of cycle number, depending upon the patient's disease and/or severity.

For the purposes of the present invention, “one or more cycles of an anti-CS1 cycle” and/or “one or more cycles of an anti-CS1 agent” means at least 1, at least 2, at least 3, at least 4, at least 5, at least 6, at least 7, at least 8, at least 9, or at least 10 cycles of primary treatment with either agent(s), followed by one or more optional maintenance cycles of either agent(s). The maintenance cycle(s) may follow a similar number of cycles as outlined for the primary therapy, or may be significantly longer or shorter in terms of cycle number, depending upon the patient's disease and/or severity.

In another aspect of the present invention, the sequential dosing regimen may comprise a “hybrid cycle” in which the patient is administered one or more anti-CS1 agent cycles, followed by one or more agonist CD137 agent cycles, followed by one or more anti-CS1 agent cycles and/or one or more agonist CD137 agent cycles.

The phrase “sequential dosing regimen”, generally refers to treating a patient with at least two agents in a specific order, wherein one cycle of a first agent is administered after the cycle of other agent. In addition, the phrase “sequential dosing regimen” also encompasses the phrase “phased dosing regimen” as it is traditionally referred to in the pharmaceutical arts. In one context, “sequential dosing regimen” refers to not only the order in which the cycles are administered, but also to the entire treatment regimen for the patient. For example, “sequential dosing regimen” may include the complete dosing regimen for the patient including one or more cycles of an anti-CS1 agent, followed by one or more cycles of either an agonist CD137 agent or a combination comprising an agonist CD137 agent and one or more anti-CS1 agent.

For the purposes of the present invention, the concurrent administration of an anti-CS1 agent with an agonist CD137 agent, or the sequential administration of an anti-CS1 agent followed by an agonist CD137 agent, may be administered after a sufficient period of time after a patients prior therapy has passed, which may be at least about 3 weeks, about 4 weeks, about 5 weeks, about 6 weeks, about 7 weeks, about 8 weeks, about 9 weeks, about 10 weeks, about 11 weeks, about 12 weeks, or more weeks after the patients prior therapy has ended and/or after the physician has determined the prior therapy had failed.

The phrase “clinical benefit” or “benefit” refers to a condition where a patient achieves a complete response; partial response; stable disease; or as otherwise described herein.

In another aspect of the present invention, the concurrent administration of an anti-CS1 agent with an agonist CD137 agent, or the sequential administration of an anti-CS1 agent followed by an agonist CD137 agent, may be administered in further combination with one or more immunomodulatory agents, co-stimulatory pathway modulators.

The phrase “immunomodulatory agent” generally refers to an agent that either increases or decreases the function of the immune system, and/or as defined elsewhere herein, and includes co-stimulatory pathway modulators, Ipilimumab; ORENCIA®; Belatacept; CD28 antagonists, CD80 antagonists, CD86 antagonists, PD1 antagonists, PDL1 antagonists, CTLA-4 antagonists, and KIR antagonists, among others disclosed herein.

The phrase “co-stimulatory pathway modulator”, generally refers to an agent that functions by increasing or decreasing the function of the immune system by modulating the co-stimulatory pathway. In one aspect of the present invention, a co-stimulatory pathway modulator is an immunostimulant or T-cell activator, and may also encompass any agent that is capable of disrupting the ability of CD28 antigen to bind to its cognate ligand, to inhibit the ability of CTLA-4 to bind to its cognate ligand, to augment T cell responses via the co-stimulatory pathway, to disrupt the ability of B7 to bind to CD28 and/or CTLA-4, to disrupt the ability of B7 to activate the co-stimulatory pathway, to disrupt the ability of CD80 to bind to CD28 and/or CTLA-4, to disrupt the ability of CD80 to activate the co-stimulatory pathway, to disrupt the ability of CD86 to bind to CD28 and/or CTLA-4, to disrupt the ability of CD86 to activate the co-stimulatory pathway, and to disrupt the co-stimulatory pathway, in general from being activated. This necessarily includes small molecule inhibitors of CD28, CD80, CD86, CTLA-4, among other members of the co-stimulatory pathway; antibodies directed to CD28, CD80, CD86, CTLA-4, among other members of the co-stimulatory pathway; antisense molecules directed against CD28, CD80, CD86, CTLA-4, among other members of the co-stimulatory pathway; adnectins directed against CD28, CD80, CD86, CTLA-4, among other members of the co-stimulatory pathway, RNAi inhibitors (both single and double stranded) of CD28, CD80, CD86, CTLA-4, among other members of the co-stimulatory pathway, among other anti-CTLA-4 antagonists.

Anti-CTLA-4 antagonist agents for use in the methods of the invention, include, without limitation, anti-CTLA-4 antibodies, human anti-CTLA-4 antibodies, mouse anti-CTLA-4 antibodies, mammalian anti-CTLA-4 antibodies, humanized anti-CTLA-4 antibodies, monoclonal anti-CTLA-4 antibodies, polyclonal anti-CTLA-4 antibodies, chimeric anti-CTLA-4 antibodies, MDX-010 (Ipilimumab), tremelimumab, anti-CD28 antibodies, anti-CTLA-4 adnectins, anti-CTLA-4 domain antibodies, single chain anti-CTLA-4 fragments, heavy chain anti-CTLA-4 fragments, light chain anti-CTLA-4 fragments, modulators of the co-stimulatory pathway, the antibodies disclosed in PCT Publication No. WO 2001/014424, the antibodies disclosed in PCT Publication No. WO 2004/035607, the antibodies disclosed in U.S. Publication No. 2005/0201994, and the antibodies disclosed in granted European Patent No. EP 1212422 B1. Additional CTLA-4 antibodies are described in U.S. Pat. Nos. 5,811,097, 5,855,887, 6,051,227, and 6,984,720; in PCT Publication Nos. WO 01/14424 and WO 00/37504; and in U.S. Publication Nos. 2002/0039581 and 2002/086014. Other anti-CTLA-4 antibodies that can be used in a method of the present invention include, for example, those disclosed in: PCT Publication No. WO 98/42752; U.S. Pat. Nos. 6,682,736 and 6,207,156; Hurwitz et al., Proc. Natl. Acad. Sci. USA, 95(17):10067-10071 (1998); Camacho et al., J. Clin. Oncology, 22(145): Abstract No. 2505 (2004) (antibody CP-675206); Mokyr et al., Cancer Res., 58:5301-5304 (1998), and U.S. Pat. Nos. 5,977,318, 6,682,736, 7,109,003, and 7,132,281. Each of these references is specifically incorporated herein by reference for purposes of description of CTLA-4 antibodies. A preferred clinical CTLA-4 antibody is human monoclonal antibody 10D1 (also referred to as MDX-010 and Ipilimumab and available from Medarex, Inc., Bloomsbury, N.J.), disclosed in PCT Publication No. WO 01/14424.

As is known in the art, Elotuzumab refers to an anti-CS1 antibody, and is a humanized antibody anti-CS1 monoclonal antibody that enhances natural killer cell mediated antibody dependent cellular cytotoxicity of CS1 expressing myeloma cells. Elotuzumab can also be referred to as BMS-901608, or by its CAS Registry No. 915296-00-3, and is disclosed as antibody HuLuc63 in PCT Publication No. WO 2004/100898, incorporated herein by reference in its entirety and for all purposes. Specifically, Elotuzumab describes a humanized monoclonal antibody or antigen-binding portion thereof that specifically binds to CS-1, comprising a light chain variable region and a heavy chain variable region having a light chain variable region comprised of SEQ ID NO:1, and comprising a heavy chain region comprised of SEQ ID NO:2, or antigen binding fragments and variants thereof. Elotuzumab may also be described as an antibody comprising a heavy chain CDR1 having amino acids 31-35 of SEQ ID NO:2: a heavy chain CDR2 having amino acids 50-66 of SEQ ID NO:2; and a heavy chain CDR3 having amino acids 99-108 of SEQ ID NO:2; in addition to a light chain CDR1 having amino acids 24-34 of SEQ ID NO:1; a light chain CDR2 having amino acids 50-56 of SEQ ID NO:1; and a light chain CDR3 having amino acids 89-97 of SEQ ID NO:1. Pharmaceutical compositions of Elotuzumab include all pharmaceutically acceptable compositions comprising Elotuzumab and one or more diluents, vehicles and/or excipients. Elotuzumab may be administered by I.V. at a dose of about 1 mg/kg, 10 mg/kg, about 20 mg/kg, or between about 10 to about 20 mg/kg.

Light chain variable region for Elotuzumab:
(SEQ ID NO: 1)
DIQMTQSPSSLSASVGDRVTITCKASQDVGIAVAWYQQKPGKVPKLLIYW
ASTRHTGVPDRFSGSGSGTDFTLTISSLQPEDVATYYCQQYSSYPYTFGQ
GTKVEIK
Heavy chain variable region for Elotuzumab:
(SEQ ID NO: 2)
EVQLVESGGGLVQPGGSLRLSCAASGFDFSRYWMSWVRQAPGKGLEWIGE
INPDSSTINYAPSLKDKFIISRDNAKNSLYLQMNSLRAEDTAVYYCARPD
GNYWYFDVWGQGTLVTVSS

As is known in the art, Urelumab refers to an anti-CD137 antibody, and is a fully human IgG₄ antibody derived from transgenic mice having human genes encoding heavy and light chains to generate a functional human repertoire. Urelumab can also be referred to as BMS-663513, or by its CAS Registry No. 934823-49-1, and is disclosed as antibody 1007 in U.S. Pat. No. 7,288,638, incorporated herein by reference in its entirety and for all purposes. Specifically, BMS-663513 describes a human monoclonal antibody or antigen-binding portion thereof that specifically binds to 4-1BB, comprising a light chain variable region provided as SEQ ID NO:3, and a heavy chain variable region provided as SEQ ID NO:4, or antigen binding fragments and variants thereof. Urelumab may also be described as an antibody comprising a light chain CDR1 having amino acids 44-54 of SEQ ID NO:3, a light chain CDR2 having amino acids 70-76 of SEQ ID NO:3, and a light chain CDR3 having amino acids 109-119 of SEQ ID NO:3; and comprising a heavy chain CDR1 having amino acids 50-54 of SEQ ID NO:4, a heavy chain CDR2 having amino acids 69-84 of SEQ ID NO:4, and a heavy chain CDR3 having amino acids 117-129 of SEQ ID NO:4. Pharmaceutical compositions of BMS-663513 include all pharmaceutically acceptable compositions comprising BMS-663513 and one or more diluents, vehicles and/or excipients. BMS-663513 may be administered by I.V.

Light chain variable region for Urelumab:
(SEQ ID NO: 3)
MEAPAQLLFLLLLWLPDTTGEIVLTQSPATLSLSPGERATLSCRASQSVS
SYLAWYQQKPGQAPRLLIYDASNRATGIPARFSGSGSGTDFTLTISSLEP
EDFAVYYCQQRSNWPPALTFGGGTKVEIKRTVAAPSVFIFPPSDEQLKSG
TASVVCLLNNFYPREAKVQWKVDNALQSGNSQESVTEQDSKDSTYSLSST
LTLSKADYEKHKVYACEVTHQGLSSPVTKSFNRGEC
Heavy chain variable region for Urelumab:
(SEQ ID NO: 4)
MKHLWFFLLLVAAPRWVLSQVQLQQWGAGLLKPSETLSLTCAVYGGSFSG
YYWSWIRQSPEKGLEWIGEINHGGYVTYNPSLESRVTISVDTSKNQFSLK
LSSVTAADTAVYYCARDYGPGNYDWYFDLWGRGTLVTVSSASTKGPSVFP
LAPCSRSTSESTAALGCLVKDYFPEPVTVSWNSGALTSGVHTFPAVLQSS
GLYSLSSVVTVPSSSLGTKTYTCNVDHKPSNTKVDKRVESKYGPPCPPCP
APEFLGGPSVFLFPPKPKDTLMISRTPEVTCVVVDVSQEDPEVQFNWYVD
GVEVHNAKTKPREEQFNSTYRVVSVLTVLHQDWLNGKEYKCKVSNKGLPS
SIEKTISKAKGQPREPQVYTLPPSQEEMTKNQVSLTCLVKGFYPSDIAVE
WESNGQPENNYKTTPPVLDSDGSFFLYSRLTVDKSRWQEGNVFSCSVMHE
ALHNHYTQKSLSLSLGK

As noted elsewhere herein, the administration of an anti-CS1 agent and/or an agonist CD137 agent, may be administered either alone or in combination with a peptide antigen (e.g., gp100). A non-limiting example of a peptide antigen would be a gp100 peptide comprising, or alternatively consisting of, the sequence selected from the group consisting of: IMDQVPFSV (SEQ ID NO:5), and YLEPGPVTV (SEQ ID NO:6). Such a peptide may be administered orally, or preferably at 1 mg emulsified in incomplete Freund's adjuvant (IFA) injected s.c. in one extremity, and 1 mg of either the same or a different peptide emulsified in IFA may be injected in another extremity.

Disorders for which the concurrent and/or sequential dosing regimens of the present invention may be useful in treating include, but are not limited to: multiple myeloma, melanoma, primary melanoma, unresectable stage III or IV malignant melanoma, lung cancer, non-small cell lung cancer, small cell lung cancer, prostate cancer; solid tumors, pancreatic cancer, prostatic neoplasms, breast cancer, neuroblastoma, kidney cancer, ovarian cancer, sarcoma, bone cancer, testicular cancer, hematopoietic cancers, leukemia, lymphoma, multiple myeloma, and myelodysplastic syndromes.

Additional disorders for which the concurrent and/or sequential dosing of the present invention may be useful in treating include, but are not limited to the following: glioma, gastrointestinal cancer, renal cancer, ovarian cancer, liver cancer, colorectal cancer, endometrial cancer, kidney cancer, thyroid cancer, neuroblastoma, pancreatic cancer, glioblastoma multiforme, cervical cancer, stomach cancer, bladder cancer, hepatoma, breast cancer, colon carcinoma, and head and neck cancer, gastric cancer, germ cell tumor, bone cancer, bone tumors, adult malignant fibrous histiocytoma of bone; childhood malignant fibrous histiocytoma of bone, sarcoma, pediatric sarcoma, sinonasal natural killer, neoplasms, plasma cell neoplasm; myelodysplastic syndromes; neuroblastoma; testicular germ cell tumor, intraocular melanoma, myelodysplastic syndromes; myelodysplastic/myeloproliferative diseases, synovial sarcoma, chronic myeloid leukemia, acute lymphoblastic leukemia, Philadelphia chromosome positive acute lymphoblastic leukemia (Ph+ALL), multiple myeloma, acute myelogenous leukemia, chronic lymphocytic leukemia, mastocytosis and any symptom associated with mastocytosis, and any metastasis thereof. In addition, disorders include urticaria pigmentosa, mastocytosises such as diffuse cutaneous mastocytosis, solitary mastocytoma in human, as well as dog mastocytoma and some rare subtypes like bullous, erythrodermic and teleangiectatic mastocytosis, mastocytosis with an associated hematological disorder, such as a myeloproliferative or myelodysplastic syndrome, or acute leukemia, myeloproliferative disorder associated with mastocytosis, mast cell leukemia, in addition to other cancers. Other cancers are also included within the scope of disorders including, but are not limited to, the following: carcinoma, including that of the bladder, urothelial carcinoma, breast, colon, kidney, liver, lung, ovary, pancreas, stomach, cervix, thyroid, testis, particularly testicular seminomas, and skin; including squamous cell carcinoma; gastrointestinal stromal tumors (“GIST”); hematopoietic tumors of lymphoid lineage, including leukemia, acute lymphocytic leukemia, acute lymphoblastic leukemia, B-cell lymphoma, T-cell lymphoma, Hodgkins lymphoma, non-Hodgkins lymphoma, hairy cell lymphoma and Burkitt's lymphoma; hematopoietic tumors of myeloid lineage, including acute and chronic myelogenous leukemias and promyelocytic leukemia; tumors of mesenchymal origin, including fibrosarcoma and rhabdomyosarcoma; other tumors, including melanoma, seminoma, tetratocarcinoma, neuroblastoma and glioma; tumors of the central and peripheral nervous system, including astrocytoma, neuroblastoma, glioma, and schwannomas; tumors of mesenchymal origin, including fibrosarcoma, rhabdomyosarcoma, and osteosarcoma; and other tumors, including melanoma, xenoderma pigmentosum, keratoactanthoma, seminoma, thyroid follicular cancer, teratocarcinoma, chemotherapy refractory non-seminomatous germ-cell tumors, and Kaposi's sarcoma, and any metastasis thereof.

The terms “treating”, “treatment” and “therapy” as used herein refer to curative therapy, prophylactic therapy, preventative therapy, and mitigating disease therapy.

The phrase “more aggressive dosing regimen” or “increased dosing frequency regimen”, as used herein refers to a dosing regimen that necessarily exceeds the basal and/or prescribed dosing regimen of either the anti-CS1 agent arm of the dosing regimen and/or the agonist CD137 arm of the dosing regimen, either due to an increased dosing frequency (about once a week, about bi-weekly, about once daily, about twice daily, etc.), increased or escalated dose (in the case of the anti-CS1 antibody: about 11, about 12, about 13, about 14, about 15, about 16, about 17, about 18, about 19, about 20, about 21, about 22, about 23, about 24, about 25, about 26, about 27, about 28, about 29, about 30, about 35, about 40 mg/kg; or in the case of the anti-CD137 antibody: about 0.01 mg/kg, about 0.02 mg/kg, about 0.03 mg/kg, about 0.05 mg/kg, about 0.075 mg/kg, about 0.1 mg/kg, about 0.2 mg/kg, about 0.3 mg/kg, about 0.4 mg/kg, about 0.5 mg/kg, about 0.6 mg/kg, about 0.7 mg/kg, about 0.8 mg/kg, about 0.9 mg/kg, about 1 mg/kg, about 1.2 mg/kg, about 1.4 mg/kg, about 1.6 mg/kg, about 1.8 mg/kg, or about 2.0 mg/kg; or about 1 mg, about 2 mg, about 3 mg, about 4 mg, about 5 mg, about 6 mg, about 7 mg, about 8 mg, about 9, about 10 mg, about 11 mg, about 12 mg, about 13 mg, about 14 mg, about 15 mg, or about 16 mg), or by changing the route of administration which may result in an increased, bio-available level of said anti-CS1 agent and/or said the agonist CD137 agent.

It is to be understood this invention is not limited to particular methods, reagents, compounds, compositions, or biological systems, which can, of course, vary. It is also to be understood that the terminology used herein is for the purpose of describing particular aspects only, and is not intended to be limiting.

As used in this specification and the appended claims, the singular forms “a”, “an”, and “the” include plural referents unless the content clearly dictates otherwise. Thus, for example, reference to “a peptide” includes a combination of two or more peptides, and the like.

“About” as used herein when referring to a measurable value such as an amount, a temporal duration, and the like, is meant to encompass variations of ±20% or ±10%, preferably ±5%, or ±1%, or as little as ±0.1% from the specified value, as such variations are appropriate to perform the disclosed methods, unless otherwise specified herein.

As used herein; the terms CS1, SLAMF7, SLAM Family Member 7, CD2 Subset, CRACC, CD2-Like Receptor-Activating Cytotoxic Cells, 19A24 Protein, 19A, CD2-Like Receptor Activating Cytotoxic Cells, CD319, Novel LY9 (Lymphocyte Antigen 9) Like Protein, Membrane Protein FOAP-12, CD319 Antigen, Protein 19A, APEX-1, FOAP12, and Novel Ly93 are used interchangeably, and include variants, isoforms, species homologs of human CS1, and analogs having at least one common epitope with CS1.

CS1 is a cell surface glycoprotein that is highly expressed on Multiple Myeloma cells. CS1 is characterized by two extracellular immunoglobulin (Ig)-like domains and an intracellular signaling domain with immune receptor tyrosine-based switch motifs (Tai, Y.-T. et al., Blood, 113(18):4309-4318 (Apr. 30, 2009); Bhat, R. et al., J. Leukoc. Biol., 79:417-424 (2006); Fischer, A. et al., Curr. Opin. Immunol., 19:348-353 (2007); Boles, K. S. et al., Immunogenetics, 52:302-307 (2001); Lee, J. K. et al., J. Immunol., 179:4672-4678 (2007); and Veillette, A., Immunol. Rev., 214:22-34 (2006)). CS1 is expressed at high levels in normal and malignant plasma cells, but not normal organs, solid tumors, or CD34⁺ stem cells. Only a small subset of resting lymphocytes, including NK cells and a subset of CD8⁺ T cells, express detectable but low levels of CS1 (His, E. D. et al., Clin. Cancer Res., 14:2775-2784 (2008) and Murphy, J. J. et al., Biochem. J., 361:431-436 (2002)).

CS1 was isolated and cloned by Boles et al. (Immunogenetics, 52(3-4):302-307 (2001)). The complete CS1 sequence can be found under GENBANK® Accession No. NM_021181.3 and is as follows:

(SEQ ID NO: 7)
MAGSPTCLTLIYILWQLTGSAASGPVKELVGSVGGAVTFPLKSKVKQVDS
IVWTFNTTPLVTIQPEGGTIIVTQNRNRERVDFPDGGYSLKLSKLKKNDS
GIYYVGIYSSSLQQPSTQEYVLHVYEHLSKPKVTMGLQSNKNGTCVTNLT
CCMEHGEEDVIYTWKALGQAANESHNGSILPISWRWGESDMTFICVARNP
VSRNFSSPILARKLCEGAADDPDSSMVLLCLLLVPLLLSLFVLGLFLWFL
KRERQEEYIEEKKRVDICRETPNICPHSGENTEYDTIPHTNRTILKEDPA
NTVYSTVEIPKI(MENPHSLLTMPDTPRLFAYENV

As used herein, the terms CD137, 41-BB, Ly63; CD137 antigen; TNFR9, TNFRSF9, and tumor necrosis factor receptor superfamily member 9, are used interchangeably, and include variants, isoforms, species homologs of human CD137, and analogs having at least one common epitope with CD137.

CD137 is a member of the TNF-receptor superfamily. This receptor contributes to the clonal expansion, survival, and development of T cells. It can also induce proliferation in peripheral monocytes, enhance T cell apoptosis induced by TCR/CD3 triggered activation, and regulate CD28 co-stimulation to promote Th1 cell responses. The expression of this receptor is induced by lymphocyte activation. TRAF adaptor proteins have been shown to bind to this receptor and transduce the signals leading to activation of NF-κB.

CD137, also known as an inducible T cell surface molecule, is a 30 kDa glycoprotein of the tumor necrosis factor (TNF) receptor superfamily. Its alternative names are TNF receptor superfamily member 9 (TNFRSF9), and 4-1BB, and it is induced by lymphocyte activation. It is mainly expressed on activated CD4+ and CD8+ T cells, activated B cells, and natural killer (NK) cells but can also be found on resting monocytes and dendritic cells. As a costimulatory molecule, CD137 is involved in the activation and survival of CD4+, CD8+, and NK cells. Its engagement with anti-CD137 monoclonal antibody enhances the expansion of T cells and activates them to secrete cytokines.

Treatment of tumor-bearing mice with immune costimulatory anti-CD137 has been found to significantly reduce the tumor burden. Cell-depletion studies have shown that the antitumor effects of anti-CD137 depend on CD8+ T cells and not on CD4+ T cells or NK cells. Further analysis revealed that this tumor regression was correlated with increased numbers of lymphocytes in the mice spleens and tumor-draining lymph nodes, and increased numbers of apoptotic cells and TILs. Furthermore, mice that received this combination therapy rapidly rejected tumors when rechallenged, suggesting that long-lasting tumor antigen-specific memory had been established (Miller et al., J. Immunol., 169(4):1792-1800 (Aug. 15, 2002); Palazon et al., Cancer Res., 71(3):801-811 (Feb. 1, 2011); Ju et al., Int. J. Cancer, 122(12):2784-2790 (2008)).

The complete CD137 sequence can be found under GENBANK® Accession No. NP_001552 and is as follows:

(SEQ ID NO: 8)
MGNSCYNIVATLLLVLNFERTRSLQDPCSNCPAGTFCDNNRNQICSPCPP
NSFSSAGGQRTCDICRQCKGVFRTRKECSSTSNAECDCTPGFHCLGAGCS
MCEQDCKQGQELTKKGCKDCCFGTFNDQKRGICRPWTNCSLDGKSVLVNG
TKERDVVCGPSPADLSPGASSVTPPAPAREPGHSPQIISFFLALTSTALL
FLLFFLTLRFSVVKRGRKKLLYIFKQPFMRPVQTTQEEDGCSCRFPEEEE
GGCEL

Specific concurrent and/or sequential dosing regimens for any given patient may be established based upon the specific disease for which the patient has been diagnosed, or in conjunction with the stage of the patient's disease. For example, if a patient is diagnosed with a less-aggressive cancer, or a cancer that is in its early stages, the patient may have an increased likelihood of achieving a clinical benefit and/or immune-related response to a concurrent administration of an anti-CS1 agent followed by an agonist CD137 agent and/or a sequential administration of an anti-CS1 agent followed by an agonist CD137 agent. Alternatively, if a patient is diagnosed with a more-aggressive cancer, or a cancer that is in its later stages, the patient may have a decreased likelihood of achieving a clinical benefit and/or immune-related response to said concurrent and/or sequential administration, and thus may suggest that either higher doses of said anti-CS1 agent and/or said agonist CD137 agent therapy should be administered or more aggressive dosing regimens or either agent or combination therapy may be warranted. In one aspect, an increased dosing level of a anti-CS1, such as Ipilimumab, would be about 10, 20, 30, 40, 50, 60, 70, 80, 90, or 95% more than the typical anti-CS1 agent dose for a particular indication or individual (e.g., about 0.3 mg/kg, about 1 mg/kg, about 3 mg/kg, about 10 mg/kg, about 15 mg/kg, about 20 mg/kg, about 25 mg/kg, about 30 mg/kg), or about 1.5×, 2×, 2.5×, 3×, 3.5×, 4×, 4.5×, 5×, 6×, 7×, 8×, 9×, or 10× more anti-CS1 agent than the typical dose for a particular indication or for individual. In another aspect, an increased dosing level of an agonist CD137 agent would be about 10, 20, 30, 40, 50, 60, 70, 80, 90, or 95% more than the typical agonist CD137 agent dose for a particular indication or individual (e.g., about 0.03 mg/kg, 0.1 mg/kg, 0.3 mg/kg, about 3 mg/kg, about 10 mg/kg, about 15 mg/kg, about 20 mg/kg, about 25 mg/kg, about 30 mg/kg; or about 3 mg, about 4 mg, about 5 mg, about 6 mg, about 7 mg, about 8 mg, about 9 mg, about 10 mg, about 11 mg, about 12 mg, about 13 mg, about 14 mg, about 15 mg or about 16 mg), or about 1.5×, 2×, 2.5×, 3×, 3.5×, 4×, 4.5×, 5×, 6×, 7×, 8×, 9×, or 10× more agonist CD137 agent than the typical dose for a particular indication or for individual.

A therapeutically effective amount of an anti-CS1 agent and/or an agonist CD137 agent, can be orally administered if it is a small molecule modulator, for example, or preferably injected into the patient, for example if it is a biologic agent. The actual dosage employed can be varied depending upon the requirements of the patient and the severity of the condition being treated. Determination of the proper starting dosage for a particular situation is within the skill of the art, though the assignment of a treatment regimen will benefit from taking into consideration the indication and the stage of the disease. Nonetheless, it will be understood that the specific dose level and frequency of dosing for any particular patient can be varied and will depend upon a variety of factors including the activity of the specific compound employed, the metabolic stability and length of action of that compound, the species, age, body weight, general health, sex and diet of the patient, the mode and time of administration, rate of excretion, drug combination, and severity of the particular condition. Preferred patients for treatment include animals, most preferably mammalian species such as humans, and domestic animals such as dogs, cats, and the like, patient to cancer.

As used herein, the terms “induction” and “induction phase” are used interchangeably and refer to the first phase of treatment in the clinical trial. For example, during induction, subjects may receive intravenous doses of an agonistic CD137 antibody in combination with an anti-CS1 antibody.

As used herein, the terms “maintenance” and “maintenance phase” are used interchangeably and refer to the second phase of treatment in the clinical trial. For example, during maintenance, subjects may receive an agonistic CD137 in combination with an anti-CS1 antibody. In certain embodiments, treatment is continued as long as clinical benefit is observed or until unmanageable toxicity or disease progression occurs.

As used herein, the terms “fixed dose”, “flat dose” and “flat-fixed dose” are used interchangeably and refer to a dose that is administered to a patient without regard for the weight or body surface area (BSA) of the patient. The fixed or flat dose is therefore not provided as a mg/kg dose, but rather as an absolute amount of the agent (e.g., the agonistic CD137 antibody and/or anti-CS1 antibody).

As used herein, a “body surface area (BSA)-based dose” refers to a dose (e.g., of the agonistic CD137 antibody and/or anti-CS1 antibody) that is adjusted to the body-surface area (BSA) of the individual patient. A BSA-based dose may be provided as mg/kg body weight. Various calculations have been published to arrive at the BSA without direct measurement, the most widely used of which is the Du Bois formula (see Du Bois, D. et al., Archives of Internal Medicine, 17(6):863-871 (June 1916); and Verbraecken, J. et al., Metabolism—Clinical and Experimental, 55(4):515-4 (April 2006)). Other exemplary BSA formulas include the Mosteller formula (Mosteller, R. D., N. Engl. J. Med., 317:1098 (1987)), the Haycock formula (Haycock, G. B. et al., J. Pediatr., 93:62-66 (1978)), the Gehan and George formula (Gehan, E. A. et al., Cancer Chemother. Rep., 54:225-235 (1970)), the Boyd formula (Current, J. D., The Internet Journal of Anesthesiology, 2(2) (1998); and Boyd, E., University of Minnesota, The Institute of Child Welfare, Monograph Series, No. 10., Oxford University Press, London (1935)), the Fujimoto formula (Fujimoto, S. et al., Nippon Eiseigaku Zasshi, 5:443-450 (1968)), the Takahira formula (Fujimoto, S. et al., Nippon Eiseigaku Zasshi, 5:443-450 (1968)), and the Schlich formula (Schlich, E. et al., Ernährungs Umschau, 57:178-183 (2010)).

The terms “combination” and “combinations” as used herein refer to either the concurrent administration of an anti-CS1 agent and an agonist CD137 agent; or to the sequential administration of an anti-CS1 agent with an agonist CD137 agent; or to the sequential administration of an agonist CD137 with an anti-CS1 agent; or to a more complex, combination, which may include for example, the combination of either an anti-CS1 agent and/or an agonist CD137 agent with another agent, such as an immunotherapeutic agent or co-stimulatory pathway modulator, preferably an agonist (i.e., immunostimulant), PROVENGE®, a tubulin stabilizing agent (e.g., pacitaxol, epothilone, taxane, etc.), Bevacizumab, IXEMPRA®, Dacarbazine, PARAPLATIN®, Docetaxel, one or more peptide vaccines, MDX-1379 Melanoma Peptide Vaccine, one or more gp100 peptide vaccine, fowlpox-PSA-TRICOM™ vaccine, vaccinia-PSA-TRICOM™ vaccine, MART-1 antigen, sargramostim, ticilimumab, Combination Androgen Ablative Therapy; the combination with a co-stimulatory pathway modulator; the combination with a tubulin stabilizing agent (e.g., pacitaxol, epothilone, taxane, etc.); the combination with IXEMPRA®, the combination with Dacarbazine, the combination with PARAPLATIN®, the combination of Ipilimumab with Docetaxel, the combination with one or more peptide vaccines, the combination with MDX-1379 Melanoma Peptide Vaccine, the combination with one or more gp100 peptide vaccine, the combination with fowlpox-PSA-TRICOM™ vaccine, the combination with vaccinia-PSA-TRICOM™ vaccine, the combination with MART-1 antigen, the combination with sargramostim, the combination with ticilimumab, and/or the combination with Combination Androgen Ablative Therapy. The combinations of the present invention may also be used in conjunction with other well known therapies that are selected for their particular usefulness against the condition that is being treated. Such combinations may provide therapeutic options to those patients who present with more aggressive indications.

In another embodiment of the present invention, the combination between an agonist CD137 agent and/or anti-CS1 agent, and at least one other agent may comprise the following: agatolimod, belatacept, blinatumomab, CD40 ligand, anti-B7-1 antibody, anti-B7-2 antibody, anti-B7-H4 antibody, AG4263, eritoran, anti-CD137 monoclonal antibodies, anti-OX40 antibody, ISF-154, and SGN-70.

In another embodiment of the present invention, the combination between an agonist CD137 agent and/or anti-CS1 agent, and at least one other agent may comprise a chemotherapeutic agent.

A variety of chemotherapeutics are known in the art, some of which are described herein. One type of chemotherapeutic is referred to as a metal coordination complex. It is believed this type of chemotherapeutic forms predominantly inter-strand DNA cross links in the nuclei of cells, thereby preventing cellular replication. As a result, tumor growth is initially repressed, and then reversed. Another type of chemotherapeutic is referred to as an alkylating agent. These compounds function by inserting foreign compositions or molecules into the DNA of dividing cancer cells. As a result of these foreign moieties, the normal functions of cancer cells are disrupted and proliferation is prevented. Another type of chemotherapeutic is an antineoplastic agent. This type of agent prevents, kills, or blocks the growth and spread of cancer cells. Still other types of anticancer agents include nonsteroidal aromatase inhibitors, bifunctional alkylating agents, etc.

In another embodiment of the present invention, the chemotherapeutic agent may comprise microtubule-stabilizing agents, such as ixabepilone (IXEMPRA®) and paclitaxel (TAXOL®), which commonly are used for the treatment of many types of cancer and represent an attractive class of agents to combine with CTLA-4 blockade.

The phrase “microtubulin modulating agent” is meant to refer to agents that either stabilize microtubulin or destabilize microtubulin synthesis and/or polymerization.

One microtubulin modulating agent is paclitaxel (marketed as TAXOL®), which is known to cause mitotic abnormalities and arrest, and promotes microtubule assembly into calcium-stable aggregated structures resulting in inhibition of cell replication.

Epothilones mimic the biological effects of TAXOL®, (Bollag et al., Cancer Res., 55:2325-2333 (1995), and in competition studies act as competitive inhibitors of TAXOL® binding to microtubules. However, epothilones enjoy a significant advantage over TAXOL® in that epothilones exhibit a much lower drop in potency compared to TAXOL® against a multiple drug-resistant cell line (Bollag et al. (1995)). Furthermore, epothilones are considerably less efficiently exported from the cells by P-glycoprotein than is TAXOL® (Gerth et al. (1996)). Additional examples of epothilones are provided in co-owned, PCT Application No. PCT/US2009/030291, filed Jan. 7, 2009, which is hereby incorporated by reference herein in its entirety for all purposes.

Ixabepilone is a semi-synthetic lactam analogue of patupilone that binds to tubulin and promotes tubulin polymerization and microtubule stabilization, thereby arresting cells in the G2/M phase of the cell cycle and inducing tumor cell apoptosis.

Additional examples of microtubule modulating agents useful in combination with immunotherapy include, but are not limited to, allocolchicine (NSC 406042), Halichondrin B (NSC 609395), colchicine (NSC 757), colchicine derivatives (e.g., NSC 33410), dolastatin 10 (NSC 376128), maytansine (NSC 153858), rhizoxin (NSC 332598), paclitaxel (TAXOL®, NSC 125973), TAXOL® derivatives (e.g., derivatives (e.g., NSC 608832), thiocolchicine NSC 361792), trityl cysteine (NSC 83265), vinblastine sulfate (NSC 49842), vincristine sulfate (NSC 67574), natural and synthetic epothilones including but not limited to epothilone A, epothilone B, epothilone C, epothilone D, desoxyepothilone A, desoxyepothilone B, [1S-[1R*,3R*(E),7R*,10 S*,11R*,12R*,16S*]]-7-11-dihydroxy-8,8,10,12,16-pentamethyl-3-[1-methyl-2-(2-methyl-4-thiazolyl)ethenyl]-4-aza-17 oxabicyclo [14.1.0]heptadecane-5,9-dione (disclosed in U.S. Pat. No. 6,262,094, issued Jul. 17, 2001), [1S-[1R*,3R*(E),7R*,10S*,11R*,12R*,16S*]]-3-[2-[2-(aminomethyl)-4-thiazolyl]-1-methylethenyl]-7,11-dihydroxy-8,8,10,12,16-pentamethyl-4-17-dioxabicyclo[14.1.0]-heptadecane-5,9-dione (disclosed in U.S. patent application Ser. No. 09/506,481 filed on Feb. 17, 2000, and Examples 7 and 8 herein), and derivatives thereof; and other microtubule-disruptor agents. Additional antineoplastic agents include, discodermolide (see Service, Science, 274:2009 (1996)) estramustine, nocodazole, MAP4, and the like. Examples of such agents are also described in the scientific and patent literature, see, e.g., Bulinski, J. Cell Sci., 110:3055-3064 (1997); Panda, Proc. Natl. Acad. Sci. USA, 94:10560-10564 (1997); Muhlradt, Cancer Res., 57:3344-3346 (1997); Nicolaou, Nature, 387:268-272 (1997); Vasquez, Mol. Biol. Cell., 8:973-985 (1997); and Panda, J. Biol. Chem., 271:29807-29812 (1996).

The following sets forth preferred therapeutic combinations and exemplary dosages for use in the methods of the present invention.

| Concurrent Therapeutic | Dosage | |
|---|---|---|
| Combination(s) | mg/kg (per dose) | |
| Anti-CS1 antibody + | 1-10 | mg/kg |
| Agonist-CD137 Antibody | 0.1-1 | mg/kg |
| Anti-CS1 antibody + | 0.1-1 | mg/kg |
| Agonist-CD137 Antibody | 0.1-1 | mg/kg |
| Anti-CS1 antibody + | 0.1-1 | mg/kg |
| Agonist-CD137 Antibody | 1-10 | mg/kg |
| Anti-CS1 antibody + | 1-10 | mg/kg |
| Agonist-CD137 Antibody | 0.3 | mg/kg |
| Anti-CS1 antibody + | 1-10 | mg/kg |
| Agonist-CD137 Antibody | 1 | mg/kg |
| Anti-CS1 antibody + | 1 | mg/kg |
| Agonist-CD137 Antibody | 1-10 | mg/kg |
| Anti-CS1 antibody + | 1 | mg/kg |
| Agonist-CD137 Antibody | 0.1 | mg/kg |
| Anti-CS1 antibody + | 1 | mg/kg |
| Agonist-CD137 Antibody | 0.3 | mg/kg |
| Anti-CS1 antibody + | 1 | mg/kg |
| Agonist-CD137 Antibody | 1 | mg/kg |
| Anti-CS1 antibody + | 1 | mg/kg |
| Agonist-CD137 Antibody | 0.03 | mg/kg |
| Anti-CS1 antibody + | 10 | mg/kg |
| Agonist-CD137 Antibody | 0.03 | mg/kg |
| Anti-CS1 antibody + | 1 | mg/kg |
| Agonist-CD137 Antibody | 3 | mg |
| Anti-CS1 antibody + | 10 | mg/kg |
| Agonist-CD137 Antibody | 3 | mg |
| Anti-CS1 antibody + | 1 | mg/kg |
| Agonist-CD137 Antibody | 8 | mg |
| Anti-CS1 antibody + | 10 | mg/kg |
| Agonist-CD137 Antibody | 8 | mg |

| Sequential Therapeutic | Dosage | |
|---|---|---|
| Combination(s) | mg/m² (per dose) | |
| Anti-CS1 antibody + | 1-10 | mg/kg |
| Agonist-CD137 Antibody | 0.1-1 | mg/kg |
| Anti-CS1 antibody + | 0.1-1 | mg/kg |
| Agonist-CD137 Antibody | 0.1-1 | mg/kg |
| Anti-CS1 antibody + | 0.1-1 | mg/kg |
| Agonist-CD137 Antibody | 1-10 | mg/kg |
| Anti-CS1 antibody + | 10 | mg/kg |
| Agonist-CD137 Antibody | 0.1 | mg/kg |
| Anti-CS1 antibody + | 10 | mg/kg |
| Agonist-CD137 Antibody | 0.3 | mg/kg |
| Anti-CS1 antibody + | 10 | mg/kg |
| Agonist-CD137 Antibody | 1 | |
| Agonist-CD137 Antibody + | 1-10 | mg/kg |
| Anti-CS1 antibody | 0.1-1 | mg/kg |
| Agonist-CD137 Antibody + | 0.1-1 | mg/kg |
| Anti-CS1 antibody | 0.1-1 | mg/kg |
| Agonist-CD137 Antibody + | 0.1-1 | mg/kg |
| Anti-CS1 antibody | | |
| Agonist-CD137 Antibody + | 0.1 | mg/kg |
| Anti-CS1 antibody | 10 | mg/kg |
| Agonist-CD137 Antibody + | 0.3 | mg/kg |
| Anti-CS1 antibody | 10 | mg/kg |
| Agonist-CD137 Antibody + | 1 | mg/kg |
| Anti-CS1 antibody | 10 | mg/kg |
| Anti-CS1 antibody + | 1 | mg/kg |
| Agonist-CD137 Antibody | 0.03 | mg/kg |
| Anti-CS1 antibody + | 10 | mg/kg |
| Agonist-CD137 Antibody | 0.03 | mg/kg |
| Anti-CS1 antibody + | 1 | mg/kg |
| Agonist-CD137 Antibody | 3 | mg |
| Anti-CS1 antibody + | 10 | mg/kg |
| Agonist-CD137 Antibody | 3 | mg |
| Anti-CS1 antibody + | 1 | mg/kg |
| Agonist-CD137 Antibody | 8 | mg |
| Anti-CS1 antibody + | 10 | mg/kg |
| Agonist-CD137 Antibody | 8 | mg |

While this table provides exemplary dosage ranges of the anti-CS1 and agonistic CD137 antibodies, when formulating the pharmaceutical compositions of the invention the clinician may utilize preferred dosages as warranted by the condition of the patient being treated. For example, elotuzumab may preferably be administered at about 10 mg/kg every 3 weeks. Urelumab may preferably be administered at about 0.03, 0.1, 0.1-10 mg/kg, or 3 or 8 kg, every three weeks.

The anti-CS1 antibody may preferably be administered at about 0.1-20 mg/kg, or the maximum tolerated dose. In an embodiment of the invention, a dosage of anti-CS1 antibody is administered about every three weeks. Alternatively, the anti-CS1 antibody may be administered by an escalating dosage regimen including administering a first dosage of anti-CS1 antibody at about 1 mg/kg, a second dosage of anti-CS1 antibody at about 3 mg/kg, and a third dosage of anti-CS1 antibody at about 10 mg/kg.

In another specific embodiment, the escalating dosage regimen includes administering a first dosage of anti-CS1 antibody at about 3 mg/kg and a second dosage of anti-CS1 antibody at about 10 mg/kg.

The agonistic CD137 antibody may preferably be administered at about 0.03, 0.1-20 mg/kg, or the maximum tolerated dose. In an embodiment of the invention, a dosage of agonistic CD137 antibody is administered about every three weeks. Alternatively, the agonistic CD137 antibody may be administered by an escalating dosage regimen including administering a first dosage of agonistic CD137 antibody at about 0.1 mg/kg, a second dosage of agonistic CD137 antibody at about 0.3 mg/kg, and a third dosage of agonistic CD137 antibody at about 1 mg/kg. Alternatively, the agonistic CD137 antibody may be administered by an escalating dosage regimen including administering a first dosage of agonistic CD137 antibody at about 0.03 mg/kg, a second dosage of agonistic CD137 antibody at about 0.1 mg/kg, and a third dosage of agonistic CD137 antibody at about 0.3 mg/kg.

In another specific embodiment, the escalating dosage regimen includes administering a first dosage of agonistic CD137 antibody at about 1 mg/kg and a second dosage of agonistic CD137 antibody at about 3 mg/kg.

In another specific embodiment, the escalating dosage regimen includes administering a first dosage of agonistic CD137 antibody at about 3 mg and a second dosage of agonistic CD137 antibody at about 8 mg.

Further, the present invention provides an escalating dosage regimen, which includes administering an increasing dosage of anti-CS1 antibody about every six weeks.

In one embodiment, the anti-CS1 antibody is administered on (1) day 1, week 1, (2) day 1, week 2, (3), day 1, week 3, (4), day 1, week 4, (5) day 1, week 5, (6) day 1, week 6, (7) day 1, week 7, and (8) day 1, week 8, of the induction phase. In another embodiment, the agonistic CD137 antibody is administered on (1) day 1, week 1, (2) day 1, week 4, and (3) day 1, week 7 of the induction phase. In another embodiment, the anti-CS1 antibody is administered on (1) day 1, week 10 and (2) day 1, week 15 of the maintenance phase. In another embodiment, the agonistic CD137 antibody is administered on (1) day 1, week 10 of the maintenance phase. In another embodiment, the maintenance phase is repeated for at least 1, 2, 3, 4, 5, 6, 7, 8, 9, 10, 11, 12, 13, 14, 15, 16, 17, 18, 19, or 20 or more cycles.

The actual dosage employed may be varied depending upon the requirements of the patient and the severity of the condition being treated. Generally, treatment is initiated with smaller dosages which are less than the optimum dose of the compound. Thereafter, the dosage is increased by small amounts until the optimum effect under the circumstances is reached. For convenience, the total daily dosage may be divided and administered in portions during the day if desired. Intermittent therapy (e.g., one week out of three weeks or three out of four weeks) may also be used.

In practicing the many aspects of the invention herein, biological samples can be selected preferably from blood, blood cells (red blood cells or white blood cells). Cells from a sample can be used, or a lysate of a cell sample can be used. In certain embodiments, the biological sample comprises blood cells.

Pharmaceutical compositions for use in the present invention can include compositions comprising one or a combination of co-stimulatory pathway modulators in an effective amount to achieve the intended purpose. A therapeutically effective dose refers to that amount of active ingredient which ameliorates the symptoms or condition. Therapeutic efficacy and toxicity in humans can be predicted by standard pharmaceutical procedures in cell cultures or experimental animals, for example the ED50 (the dose therapeutically effective in 50% of the population) and LD50 (the dose lethal to 50% of the population).

A “therapeutically effective amount” of either an agonist CD137 agent or an anti-CS1 agent may range anywhere from 1 to 14 fold or more higher than the typical dose depending upon the patients indication and severity of disease. Accordingly, therapeutically relevant doses of an agonist CD137 agent or an anti-CS1 agent for any disorder disclosed herein can be, for example, about 1, 2, 3, 4, 5, 6, 7, 8, 9, 10, 11, 12, 13, 14, 15, 16, 17, 18, 19, 20, 25, 30, 35, 40, 50, 60, 70, 80, 90, 100, 125, 150, 175, 200, 225, 250, or 300 fold higher than the prescribed or standard dose. Alternatively, therapeutically relevant doses of an agonist CD137 agent or an anti-CS1 agent can be, for example, about 1.0×, about 0.9×, 0.8×, 0.7×, 0.6×, 0.5×, 0.4×, 0.3×, 0.2×, 0.1×, 0.09×, 0.08×, 0.07×, 0.06×, 0.05×, 0.04×, 0.03×, 0.02×, or 0.01×.

Disorders for which the sequential dosing regimen may be useful in treating includes one or more of the following disorders: melanoma, prostate cancer, and lung cancer, for example, also include leukemias, including, for example, chronic myeloid leukemia (CML), acute lymphoblastic leukemia, and Philadelphia chromosome positive acute lymphoblastic leukemia (Ph+ALL), squamous cell carcinoma, small-cell lung cancer, non-small cell lung cancer, glioma, gastrointestinal cancer, renal cancer, ovarian cancer, liver cancer, colorectal cancer, endometrial cancer, kidney cancer, prostate cancer, thyroid cancer, neuroblastoma, pancreatic cancer, glioblastoma multiforme, cervical cancer, stomach cancer, bladder cancer, hepatoma, breast cancer, colon carcinoma, and head and neck cancer, gastric cancer, germ cell tumor, pediatric sarcoma, sinonasal natural killer, multiple myeloma, acute myelogenous leukemia, chronic lymphocytic leukemia, mastocytosis and any symptom associated with mastocytosis. In addition, disorders include urticaria pigmentosa, mastocytosises such as diffuse cutaneous mastocytosis, solitary mastocytoma in human, as well as dog mastocytoma and some rare subtypes like bullous, erythrodermic and teleangiectatic mastocytosis, mastocytosis with an associated hematological disorder, such as a myeloproliferative or myelodysplastic syndrome, or acute leukemia, myeloproliferative disorder associated with mastocytosis, and mast cell leukemia. Various additional cancers are also included within the scope of protein tyrosine kinase-associated disorders including, for example, the following: carcinoma, including that of the bladder, breast, colon, kidney, liver, lung, ovary, pancreas, stomach, cervix, thyroid, testis, particularly testicular seminomas, and skin; including squamous cell carcinoma; gastrointestinal stromal tumors (“GIST”); hematopoietic tumors of lymphoid lineage, including leukemia, acute lymphocytic leukemia, acute lymphoblastic leukemia, B-cell lymphoma, T-cell lymphoma, Hodgkins lymphoma, non-Hodgkins lymphoma, hairy cell lymphoma and Burkitt's lymphoma; hematopoietic tumors of myeloid lineage, including acute and chronic myelogenous leukemias and promyelocytic leukemia; tumors of mesenchymal origin, including fibrosarcoma and rhabdomyosarcoma; other tumors, including melanoma, seminoma, tetratocarcinoma, neuroblastoma and glioma; tumors of the central and peripheral nervous system, including astrocytoma, neuroblastoma, glioma, and schwannomas; tumors of mesenchymal origin, including fibrosarcoma, rhabdomyosarcoma, and osteosarcoma; and other tumors, including melanoma, xenoderma pigmentosum, keratoactanthoma, seminoma, thyroid follicular cancer, teratocarcinoma, chemotherapy refractory non-seminomatous germ-cell tumors, and Kaposi's sarcoma. In certain preferred embodiments, the disorder is leukemia, breast cancer, prostate cancer, lung cancer, colon cancer, melanoma, or solid tumors. In certain preferred embodiments, the leukemia is chronic myeloid leukemia (CML), Ph+ALL, AML, imatinib-resistant CML, imatinib-intolerant CML, accelerated CML, lymphoid blast phase CML.

The terms “cancer”, “cancerous”, or “malignant” refer to or describe the physiological condition in mammals, or other organisms, that is typically characterized by unregulated cell growth. Examples of cancer include, for example, solid tumors, melanoma, leukemia, lymphoma, blastoma, carcinoma and sarcoma. More particular examples of such cancers include chronic myeloid leukemia, acute lymphoblastic leukemia, Philadelphia chromosome positive acute lymphoblastic leukemia (Ph+ALL), squamous cell carcinoma, small-cell lung cancer, non-small cell lung cancer, glioma, gastrointestinal cancer, renal cancer, ovarian cancer, liver cancer, colorectal cancer, endometrial cancer, kidney cancer, prostate cancer, thyroid cancer, neuroblastoma, pancreatic cancer, glioblastoma multiforme, cervical cancer, stomach cancer, bladder cancer, hepatoma, breast cancer, colon carcinoma, and head and neck cancer, gastric cancer, germ cell tumor, pediatric sarcoma, sinonasal natural killer, multiple myeloma, acute myelogenous leukemia (AML), and chronic lymphocytic leukemia (CML).

A “solid tumor” includes, for example, sarcoma, melanoma, colon carcinoma, breast carcinoma, prostate carcinoma, or other solid tumor cancer.

“Leukemia” refers to progressive, malignant diseases of the blood-forming organs and is generally characterized by a distorted proliferation and development of leukocytes and their precursors in the blood and bone marrow. Leukemia is generally clinically classified on the basis of (1) the duration and character of the disease—acute or chronic; (2) the type of cell involved; myeloid (myelogenous), lymphoid (lymphogenous), or monocytic; and (3) the increase or non-increase in the number of abnormal cells in the blood—leukemic or aleukemic (subleukemic). Leukemia includes, for example, acute nonlymphocytic leukemia, chronic lymphocytic leukemia, acute granulocytic leukemia, chronic granulocytic leukemia, acute promyelocytic leukemia, adult T-cell leukemia, aleukemic leukemia, a leukocythemic leukemia, basophylic leukemia, blast cell leukemia, bovine leukemia, chronic myelocytic leukemia, leukemia cutis, embryonal leukemia, eosinophilic leukemia, Gross' leukemia, hairy-cell leukemia, hemoblastic leukemia, hemocytoblastic leukemia, histiocytic leukemia, stem cell leukemia, acute monocytic leukemia, leukopenic leukemia, lymphatic leukemia, lymphoblastic leukemia, lymphocytic leukemia, lymphogenous leukemia, lymphoid leukemia, lympho sarcoma cell leukemia, mast cell leukemia, megakaryocytic leukemia, micromyeloblastic leukemia, monocytic leukemia, myeloblastic leukemia, myelocytic leukemia, myeloid granulocytic leukemia, myelomonocytic leukemia, Naegeli leukemia, plasma cell leukemia, plasmacytic leukemia, promyelocytic leukemia, Rieder cell leukemia, Schilling's leukemia, stem cell leukemia, subleukemic leukemia, and undifferentiated cell leukemia. In certain aspects, the present invention provides treatment for chronic myeloid leukemia, acute lymphoblastic leukemia, and/or Philadelphia chromosome positive acute lymphoblastic leukemia (Ph+ALL).

Provided herein are methods for treating cancer (e.g., hematological cancers, including Multiple Myeloma) in a patient comprising administering to the patient an anti-CS1 antibody and an agonistic CD137. Preferably, the combination therapy exhibits therapeutic synergy.

“Therapeutic synergy” refers to a phenomenon where treatment of patients with a combination of therapeutic agents manifests a therapeutically superior outcome to the outcome achieved by each individual constituent of the combination used at its optimum dose (Corbett, T. H. et al., Cancer Treatment Reports, 66:1187 (1982)). For example, a therapeutically superior outcome is one in which the patients either a) exhibit fewer incidences of adverse events while receiving a therapeutic benefit that is equal to or greater than that where individual constituents of the combination are each administered as monotherapy at the same dose as in the combination, or b) do not exhibit dose-limiting toxicities while receiving a therapeutic benefit that is greater than that of treatment with each individual constituent of the combination when each constituent is administered in at the same doses in the combination(s) as is administered as individual components. Accordingly, in one embodiment, administration of the agonistic CD137 and anti-CS1 antibodies has a synergistic effect on treatment compared to administration of either antibody alone.

Alternatively, the combination therapy of an anti-CS1 antibody and an agonistic CD137 may have an additive or superadditive effect on suppressing cancer (e.g., Multiple Myeloma), as compared to monotherapy with either antibody alone. By “additive” is meant a result that is greater in extent than the best separate result achieved by monotherapy with each individual component, while “superadditive” is used to indicate a result that exceeds in extent the sum of such separate results. In one embodiment, the additive effect is measured as, e.g., reduction in paraproteins, reduction of plasmacytosis, reduction of bone lesions over time, increase in overall response rate, or increase in median or overall survival.

Multiple Myeloma disease response or progression, in particular, is typically measured according to the size of reduction (or rise) in paraproteins. However, the degree of plasmacytosis in the bone marrow (increase in percentage of plasma cells in the bone marrow), progression of bone lesions, and the existence of soft tissue plasmacytomas (a malignant plasma cell tumor growing within soft tissue) are also considered (Smith, D. et al., BMJ, 346:13863 (Jun. 26, 2013)). Responses to therapy may include:

Complete Response
No detectable paraprotein and disappearance of any
soft tissue plasmacytomas and
<5% plasma cells in bone marrow.
Very Good Partial Response
Greater than 90% reduction in paraproteins or
paraproteins detectable but too low to
measure.
Partial Response
Greater than 50% reduction in paraproteins.
No Change or Stable Disease
Not meeting criteria for disease response or progression.
Progressive Disease
At least a 25% increase in paraproteins (increase of at
least 5 g/L), development of new
bone lesions or plasmacytomas, or hypercalcaemia.
(corrected serum calcium >2.65 mmol/L)

Patients treated according to the methods disclosed herein preferably experience improvement in at least one sign of Multiple Myeloma. In one embodiment, the patient treated exhibits a complete response (CR), a very good partial response (VGPR), a partial response (PR), or stable disease (SD).

In one embodiment, improvement is measured by a reduction in paraprotein and/or decrease or disappearance of soft tissue plasmacytomas. In another embodiment, lesions can be measured by radiography. In another embodiment, cytology or histology can be used to evaluate responsiveness to a therapy.

In other embodiments, administration of effective amounts of the agonistic CD137 and anti-CS1 antibody according to any of the methods provided herein produces at least one therapeutic effect selected from the group consisting of reduction in paraprotein, decrease or disappearance of soft tissue plasmacytomas, CR, VGPR, PR, or SD. In still other embodiments, the methods of treatment produce a comparable clinical benefit rate (CBR=CR+PR+SD≧6 months) better than that achieved by an agonistic CD137 or anti-CS1 antibody alone. In other embodiments, the improvement of clinical benefit rate is about 20% 20%, 30%, 40%, 50%, 60%, 70%, 80% or more compared to an agonistic CD137 or anti-CS1 antibody alone.

### Antibodies

The term “antibody” describes polypeptides comprising at least one antibody derived antigen binding site (e.g., VH/VL region or Fv, or CDR). Antibodies include known forms of antibodies. For example, the antibody can be a human antibody, a humanized antibody, a bispecific antibody, or a chimeric antibody. The antibody also can be a Fab, Fab′2, ScFv, SMIP, AFFIBODY®, nanobody, or a domain antibody. The antibody also can be of any of the following isotypes: IgG1, IgG2, IgG3, IgG4, IgM, IgA1, IgA2, IgAsec, IgD, and IgE. The antibody may be a naturally occurring antibody or may be an antibody that has been altered (e.g., by mutation, deletion, substitution, conjugation to a non-antibody moiety). For example, an antibody may include one or more variant amino acids (compared to a naturally occurring antibody) which changes a property (e.g., a functional property) of the antibody. For example, numerous such alterations are known in the art which affect, e.g., half-life, effector function, and/or immune responses to the antibody in a patient. The term antibody also includes artificial polypeptide constructs which comprise at least one antibody-derived antigen binding site.

Antibodies also include known forms of antibodies. For example, the antibody can be a human antibody, a humanized antibody, a bispecific antibody, or a chimeric antibody. The antibody also can be a Fab, Fab′2, ScFv, SMIP, AFFIBODY®, nanobody, or a domain antibody. The antibody also can be of any of the following isotypes: IgG1, IgG2, IgG3, IgG4, IgM, IgA1, IgA2, IgAsec, IgD, and IgE. The antibody may be a naturally occurring antibody or may be an antibody that has been altered (e.g., by mutation, deletion, substitution, conjugation to a non-antibody moiety). For example, an antibody may include one or more variant amino acids (compared to a naturally occurring antibody) which changes a property (e.g., a functional property) of the antibody. For example, numerous such alterations are known in the art which affect, e.g., half-life, effector function, and/or immune responses to the antibody in a patient. The term antibody also includes artificial polypeptide constructs which comprise at least one antibody-derived antigen binding site.

The concurrent dosing regimen of the present invention may include the use of antibodies as one component of the combination. For example, antibodies that specifically bind to CS-1 polypeptides, preferably Elotuzumab, or CD137, preferably Urelumab.

Alternatively, the sequential dosing regimen of the present invention may include the use of antibodies as one component of the combination. For example, antibodies that specifically bind to CS-1 polypeptides, preferably Elotuzumab, or CD137, preferably Urelumab.

The term “antibody” is also used in the broadest sense and specifically covers monoclonal antibodies, polyclonal antibodies, antibody compositions with polyepitopic specificity, bispecific antibodies, diabodies, chimeric, single-chain, and humanized antibodies, as well as antibody fragments (e.g., Fab, F(ab′)₂, and Fv), so long as they exhibit the desired biological activity. Antibodies can be labeled for use in biological assays (e.g., radioisotope labels, fluorescent labels) to aid in detection of the antibody.

Antibodies can be prepared using, for example, intact polypeptides or fragments containing small peptides of interest, which can be prepared recombinantly for use as the immunizing antigen. The polypeptide or oligopeptide used to immunize an animal can be derived from the translation of RNA or synthesized chemically, and can be conjugated to a carrier protein, if desired. Commonly used carriers that are chemically coupled to peptides include, for example, bovine serum albumin (BSA), keyhole limpet hemocyanin (KLH), and thyroglobulin. The coupled peptide is then used to immunize the animal (e.g., a mouse, a rat, or a rabbit).

The term “antigenic determinant” refers to that portion of a molecule that makes contact with a particular antibody (i.e., an epitope). When a protein or fragment of a protein is used to immunize a host animal, numerous regions of the protein can induce the production of antibodies that bind specifically to a given region or three-dimensional structure on the protein; each of these regions or structures is referred to as an antigenic determinant. An antigenic determinant can compete with the intact antigen (i.e., the immunogen used to elicit the immune response) for binding to an antibody.

The phrase “specifically binds to” refers to a binding reaction that is determinative of the presence of a target in the presence of a heterogeneous population of other biologics. Thus, under designated assay conditions, the specified binding region binds preferentially to a particular target and does not bind in a significant amount to other components present in a test sample. Specific binding to a target under such conditions can require a binding moiety that is selected for its specificity for a particular target. A variety of assay formats can be used to select binding regions that are specifically reactive with a particular analyte. Typically a specific or selective reaction will be at least twice background signal or noise and more typically more than 10 times background.

### Anti-CS1 Antibodies

Anti-human-CS1 antibodies (or VH and/or VL domains derived therefrom) suitable for use in the invention can be generated using methods well known in the art. Alternatively, art recognized anti-CS1 antibodies can be used. For example, the monoclonal antibody mAb 162 described in Bouchon et al., J. Immunol., 167:5517-5521 (2001) can be used, the teachings of which are hereby incorporated by reference herein in their entirety, and in particular, those portions directly related to this antibody. Another known CS1 antibody includes the anti-CS1 antibody described in Matthew et al. (U.S. Pat. No. 7,041,499), the teachings of which are hereby incorporated by reference herein in their entirety, and in particular, those portions directly related to this antibody. Other known CS1 antibodies include the anti-CS1 antibody, Luc 63 and other antibodies that share the same epitope, including Luc 4, Luc 12, Luc 23, Luc 29, Luc 32 and Luc 37, the anti-CS1 antibody Luc 90 and other antibodies that share the same epitope, including Luc 34, Luc 69 and Luc X, and the anti-CS1 antibodies Luc2, Luc3, Luc15, Luc22, Luc35, Luc38, Luc39, Luc56, Luc60, LucX.1, LucX.2, and PDL-241, described in Williams et al. (U.S. Pat. No. 7,709,610), the teachings of which are hereby incorporated by reference herein in their entirety, and in particular, those portions directly related to these antibodies. Antibodies that compete with any of these art-recognized antibodies for binding to CS1 also can be used.

An exemplary anti-CS1 antibody is elotuzumab (also referred to as BMS-901608 and HuLuc63) comprising heavy and light chains having the sequences shown in SEQ ID NOs:17 and 18, respectively, or antigen binding fragments and variants thereof. Elotuzumab is a humanized IgG anti-CS-1 monoclonal antibody described in PCT Publication Nos. WO 2004/100898, WO 2005/10238, WO 2008/019376, WO 2008/019378, WO 2008/019379, WO 2010/051391, WO 2011/053321, and WO 2011/053322, the teachings of which are hereby incorporated by reference. Elotuzumab is known to mediate ADCC through NK cells (van Rhee, F. et al., Mol. Cancer Ther., 8(9):2616-2624 (2009)).

In other embodiments, the antibody comprises the heavy and light chain CDRs or variable regions of elotuzumab. Accordingly, in one embodiment, the antibody comprises the CDR1, CDR2, and CDR3 domains of the VH of elotuzumab having the sequence set forth in SEQ ID NO:2, and the CDR1, CDR2 and CDR3 domains of the VL of elotuzumab having the sequences set forth in SEQ ID NO:1. In another embodiment, the antibody comprises heavy chain CDR1 having amino acids 31-35 of SEQ ID NO:2: a heavy chain CDR2 having amino acids 50-66 of SEQ ID NO:2; and a heavy chain CDR3 having amino acids 99-108 of SEQ ID NO:2; in addition to a light chain CDR1 having amino acids 24-34 of SEQ ID NO:1; a light chain CDR2 having amino acids 50-56 of SEQ ID NO:1; and a light chain CDR3 having amino acids 89-97 of SEQ ID NO:1. In another embodiment, the antibody comprises VH and/or VL regions having the amino acid sequences set forth in SEQ ID NO: 2 and/or SEQ ID NO: 1, respectively. In another embodiment, the antibody competes for binding with and/or binds to the same epitope on CS1 as the above-mentioned antibodies. In another embodiment, the antibody has at least about 90% variable region amino acid sequence identity with the above-mentioned antibodies (e.g., at least about 90%, 95% or 99% variable region identity with SEQ ID NO:2 or SEQ ID NO:1).

### Anti-CD137 Antibodies

Anti-human-CD137 antibodies (or VH and/or VL domains derived therefrom) suitable for use in the invention can be generated using methods well known in the art. Alternatively, art recognized anti-CD137 antibodies can be used. For example, Suitable CD137 agonistic agents for use in the methods of the invention, include, without limitation, anti-CD137 antibodies, human anti-CD137 antibodies, mouse anti-CD137 antibodies, mammalian anti-CD137 antibodies, humanized anti-anti-CD137 antibodies, monoclonal anti-CD137 antibodies, polyclonal anti-CD137 antibodies, chimeric anti-CD137 antibodies, anti-4-1BB antibodies, anti-CD137 adnectins, anti-CD137 domain antibodies, single chain anti-CD137 fragments, heavy chain anti-CD137 fragments, light chain anti-CD137 fragments, the antibodies disclosed in U.S. Publication No. 2005/0095244, the antibodies disclosed in issued U.S. Pat. No. 7,288,638 (such as 20H4.9-IgG4 [10C7 or BMS-663513] or 20H4.9-IgG1 [BMS-663031]); the antibodies disclosed in issued U.S. Pat. No. 6,887,673 [4E9 or BMS-554271]; the antibodies disclosed in issued U.S. Pat. No. 7,214,493; the antibodies disclosed in issued U.S. Pat. No. 6,303,121; the antibodies disclosed in issued U.S. Pat. No. 6,569,997; the antibodies disclosed in issued U.S. Pat. No. 6,905,685; the antibodies disclosed in issued U.S. Pat. No. 6,355,476; the antibodies disclosed in issued U.S. Pat. No. 6,362,325 [1D8 or BMS-469492; 3H3 or BMS-469497; or 3E1]; the antibodies disclosed in issued U.S. Pat. No. 6,974,863 (such as 53A2); or the antibodies disclosed in issued U.S. Pat. No. 6,210,669 (such as 1D8, 3B8, or 3E1), and the CD137 agonistic antibodies described in U.S. Pat. Nos. 5,928,893, 6,303,121 and 6,569,997, the teachings of which are hereby incorporated by reference herein in their entirety, and in particular, those portions directly related to these antibodies. Antibodies that compete with any of these art-recognized antibodies for binding to CS1 also can be used.

An exemplary anti-CD137 antibody is urelumab (also referred to as BMS-663513) comprising heavy and light chains having the sequences shown in SEQ ID NOs:4 and 3, respectively, or antigen binding fragments and variants thereof. Urelumab is a fully human IgG4 anti-CD137 monoclonal antibody disclosed as antibody 1007 in U.S. Pat. No. 7,288,638, the teachings of which are hereby incorporated by reference. Urelumab is known to augment cellular immune responses against tumors (Melero, I. et al., Trends Pharmacol. Sci., 29(8):383-390 (2008)).

In other embodiments, the antibody comprises the heavy and light chain CDRs or variable regions of urelumab. Accordingly, in one embodiment, the antibody comprises the CDR1, CDR2, and CDR3 domains of the VH of urelumab having the sequence set forth in SEQ ID NO:4, and the CDR1, CDR2 and CDR3 domains of the VL of urelumab having the sequences set forth in SEQ ID NO:3 urelumab. In another embodiment, the antibody comprises a light chain CDR1 having amino acids 44-54 of SEQ ID NO:3, a light chain CDR2 having amino acids 70-76 of SEQ ID NO:3, and a light chain CDR3 having amino acids 109-119 of SEQ ID NO:3; and comprising a heavy chain CDR1 having amino acids 50-54 of SEQ ID NO:4, a heavy chain CDR2 having amino acids 69-84 of SEQ ID NO:4, and a heavy chain CDR3 having amino acids 117-129 of SEQ ID NO:4. In another embodiment, the antibody comprises VH and/or VL regions having the amino acid sequences set forth in SEQ ID NO: 4 and/or SEQ ID NO: 3, respectively. In another embodiment, the antibody competes for binding with and/or binds to the same epitope on CD137 as the above-mentioned antibodies. In another embodiment, the antibody has at least about 90% variable region amino acid sequence identity with the above-mentioned antibodies (e.g., at least about 90%, 95% or 99% variable region identity with SEQ ID NO:4 or SEQ ID NO:3).

### Kits

For use in the diagnostic and therapeutic applications described or suggested above, kits are also provided by the invention. Such kits can, for example, comprise a carrier means being compartmentalized to receive in close confinement one or more container means such as vials, tubes, and the like, each of the container means comprising one of the separate elements to be used in the method. For example, one of the container means can comprise one or more vials containing a pharmaceutically acceptable amount of an anti-CS1 antibody, and/or an agonistic CD137 antibody.

The kit of the invention will typically comprise the container described above and one or more other containers comprising materials desirable from a commercial and user standpoint, including buffers, diluents, filters, needles, syringes, and package inserts with instructions for use. A label can be present on the container to indicate that the composition is used for a specific therapy or non-therapeutic application, and can also indicate directions for either in vivo or in vitro use, such as those described above.

In addition, the kits can include instructional materials containing directions (i.e., protocols) for the practice of the methods of this invention. While the instructional materials typically comprise written or printed materials they are not limited to such. Any medium capable of storing such instructions and communicating them to an end user is contemplated by this invention. Such media include, but are not limited to electronic storage media (e.g., magnetic discs, tapes, cartridges, chips, and the like), optical media (e.g., CD ROM), and the like. Such media can include addresses to internet sites that provide such instructional materials.

The kit can also comprise, for example, a means for obtaining a biological sample from an individual. Means for obtaining biological samples from individuals are well known in the art, e.g., catheters, syringes, and the like, and are not discussed herein in detail.

Also provided herein are kits which include a pharmaceutical composition containing an agonistic CD antibody, such as urelumab, and an anti-CS1 antibody, such as elotuzumab, and a pharmaceutically-acceptable carrier, in a therapeutically effective amount adapted for use in the preceding methods. The kits optionally also can include instructions, e.g., comprising administration schedules, to allow a practitioner (e.g., a physician, nurse, or patient) to administer the composition contained therein to administer the composition to a patient having cancer (e.g., a hematological cancer, such as Multiple Myeloma). The kit also can include a syringe.

Optionally, the kits include multiple packages of the single-dose pharmaceutical compositions each containing an effective amount of the agonistic CD137 or anti-CS1 antibody for a single administration in accordance with the methods provided above. Instruments or devices necessary for administering the pharmaceutical composition(s) also may be included in the kits. For instance, a kit may provide one or more pre-filled syringes containing an amount of the agonistic CD137 or anti-CS1 antibody.

In one embodiment, the present invention provides a kit for treating a cancer (e.g., a hematological cancer, such as Multiple Myeloma) in a human patient, the kit comprising:

(a) a dose of an agonistic CD137 antibody comprising the CDR1, CDR2 and CDR3 domains in a heavy chain variable region comprising the sequence set forth in SEQ ID NO:3, and the CDR1, CDR2 and CDR3 domains in a light chain variable region comprising the sequence set forth in SEQ ID NO:3;

(b) a dose of an anti-CS1 antibody comprising the CDR1, CDR2 and CDR3 domains in a heavy chain variable region comprising the sequence set forth in SEQ ID NO:2, and the CDR1, CDR2 and CDR3 domains in a light chain variable region comprising the sequence set forth in SEQ ID NO:11; and

(c) instructions for using the agonistic CD137 antibody and anti-CS1 antibody in the methods described herein.

The present invention is not to be limited in scope by the embodiments disclosed herein, which are intended as single illustrations of individual aspects of the invention, and any that are functionally equivalent are within the scope of the invention. Various modifications to the models and methods of the invention, in addition to those described herein, will become apparent to those skilled in the art from the foregoing description and teachings, and are similarly intended to fall within the scope of the invention. Such modifications or other embodiments can be practiced without departing from the true scope and spirit of the invention.

The following representative Examples contain important additional information, exemplification and guidance which can be adapted to the practice of this invention in its various embodiments and the equivalents thereof. These examples are intended to help illustrate the invention, and are not intended to, nor should they be construed to, limit its scope.

### REFERENCES

1) Houot, R. et al., “Boosting antibody-dependant cellular cytotoxicity against tumor cells with a CD137 stimulatory antibody”, Oncoimmunology, 1(6):957-958 (Sep. 1, 2012).
2) Kohrt, H. E. et al., “Stimulation of natural killer cells with a CD137-specific antibody enhances trastuzumab efficacy in xenotransplant models of breast cancer”, J. Clin. Invest., 122(3):1066-75 (Mar. 1, 2012), doi: 10.1172/JCI61226.
3) Kohrt, H. E. et al., “CD137 stimulation enhances the antilymphoma activity of anti-CD20 antibodies”, Blood, 117(8):2423-2432 (Feb. 24, (2011)), doi: 10.1182/blood-2010-08-301945.

### EXAMPLES

### Example 1

### Methods for Assessing the Therapeutic Effect of Combining Elotuzumab with CD137 Agonistic mAb in an OMP-2 Multiple Myeloma Tumor Mouse Model

It has previously been shown that engagement of human immunoglobulins of the G1 isotype with FcγR expressed on NK cells or macrophages result in upregulation of CD137. Exposure of these cells to an agonist CD137 mAb such as BMS-663513, results in increased antibody dependent cellular cytotoxicity (ADCC) of target cells and enhanced efficacy in preclinical mouse models. (Houot et al., Oncoimmunology, 1(6):957-958 (2012); Kohrt et al., J. Clin. Invest., 122(3):1066-1075 (2012), doi: 10.1172/JCI61226; and Kohrt et al., Blood, 117(8):2423-32 (2011), doi: 10.1182/blood-2010-08-301945).

Elotuzumab is an antibody that selectively binds the CS1 antigen commonly expressed in human myeloma. The Fc portion of Elotuzumab, a human IgG1 antibody, has the ability to bind to Fc gamma receptors (FcγR) expressed on effector cells (NK cell and macrophages). CD137 agonism has been shown to enhance ADCC activity elicited by anti-tumor antibodies of the IgG1 isotype. The present inventors hypothesized that similar effects may be observed in combination with elotuzumab (human IgG1) against multiple myeloma which may translate into increased efficacy in preclinical tumor models.

In addition, the studies presented here evaluated various schedules of administration and dose levels to identify the best schedule(s) and dose level(s) for further evaluation in proposed human clinical trials.

### Methods

Study Design: Preclinical studies were conducted in female SCID mice (6-8 weeks old) implanted SC (subcutaneous implantation) with the multiple myeloma cell line OPM-2 (1×10⁶ cells per mouse, mixed 1:1 with MATRIGEL®). On day 8, mice were randomized into 10 experimental groups of 8 mice with a mean tumor volume of 50-60 mm³. Elotuzumab was administered IP (intraperitoneal administration) at 20 or 100 micrograms (μg) per mouse (1 or 5 mg/kg) on Day 8 (single dose, QD); CD137 mAb (BMS-469492) was administered at 100 μg per mouse, IP, every 7 days for 3 doses (Q7D×3), starting on the same day as elotuzumab, or 1 day before or 1 day after elotuzumab treatment. BMS-469492 is an anti-mouse CD137 specific antibody and was used as a surrogate for the anti-human CD137 specific monoclonal antibody (BMS-663513).

### Results

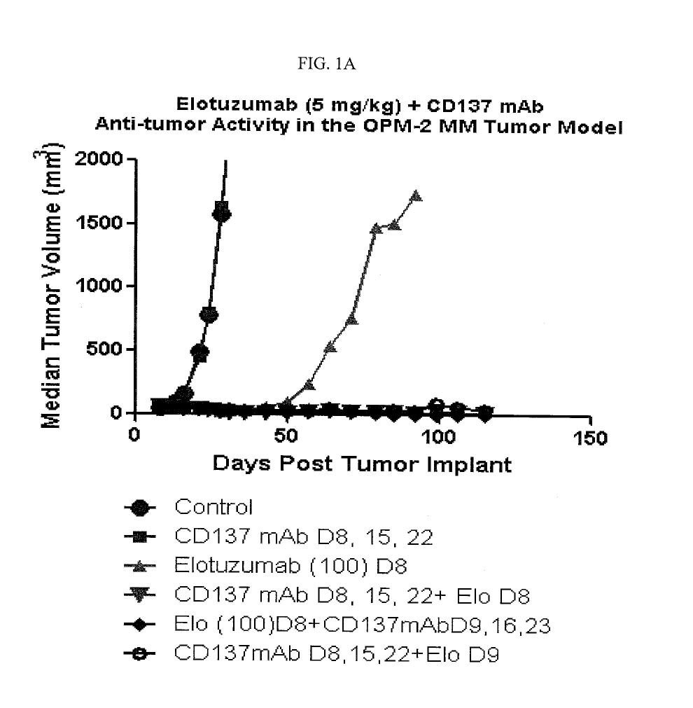

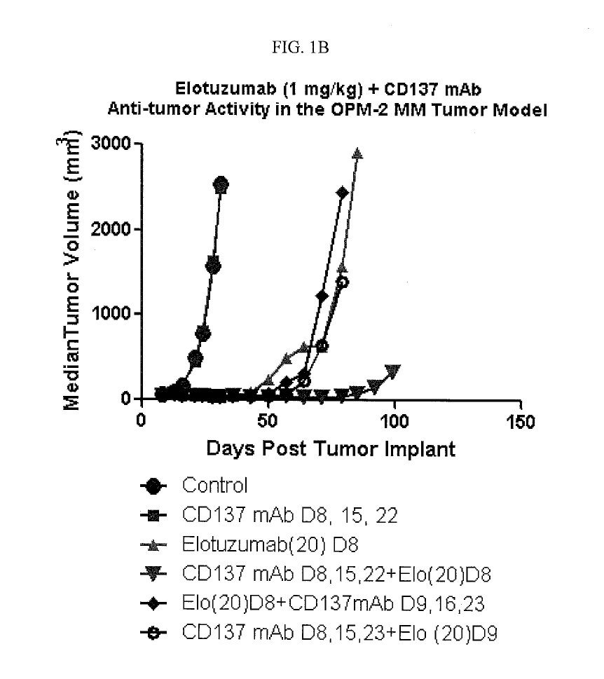

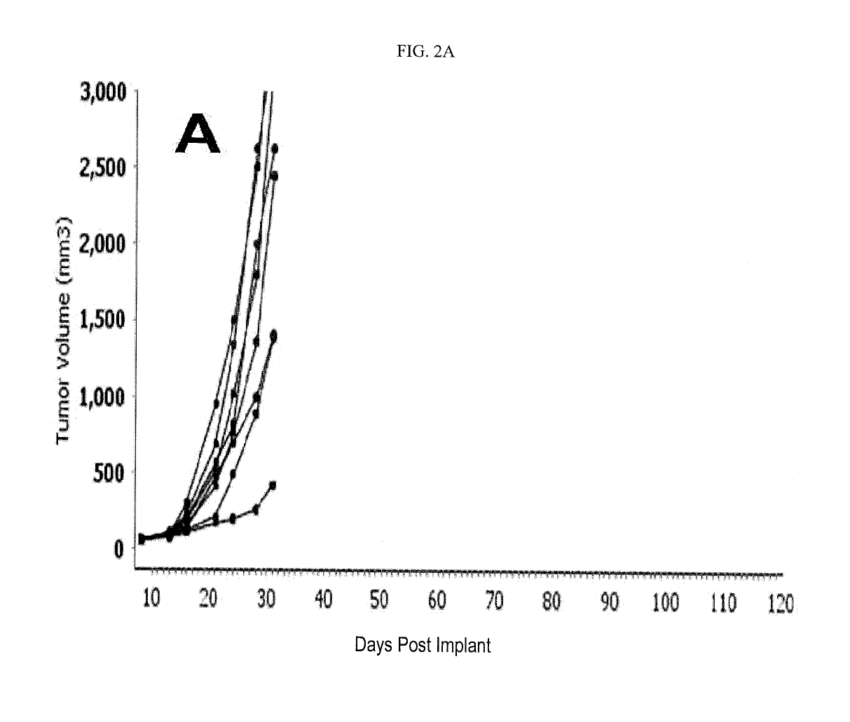

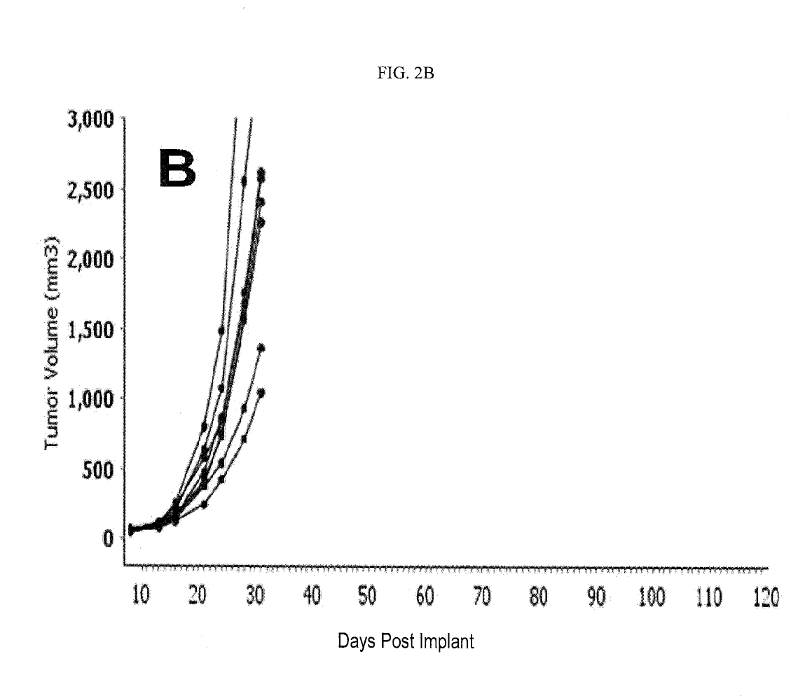

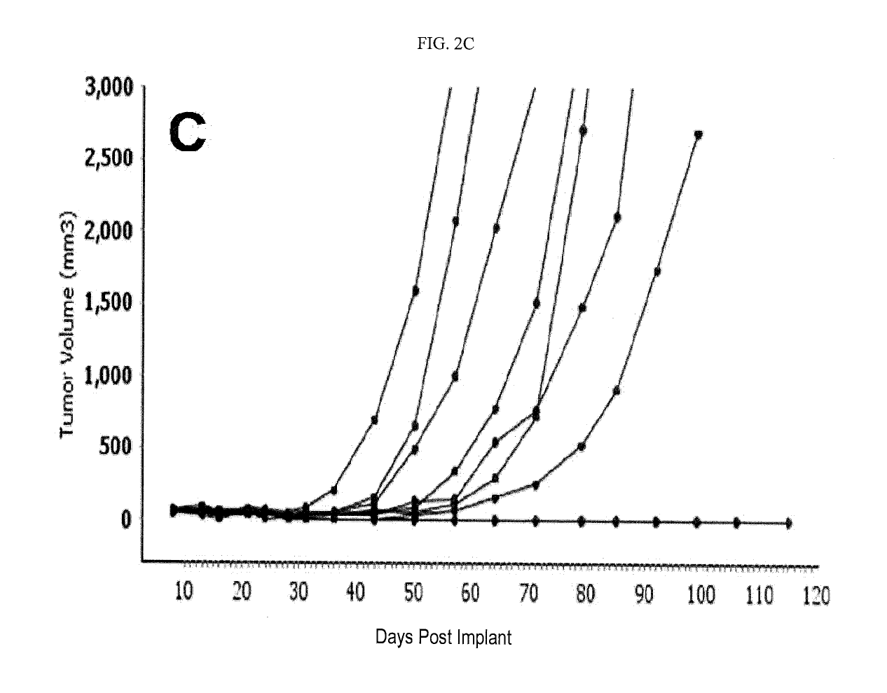

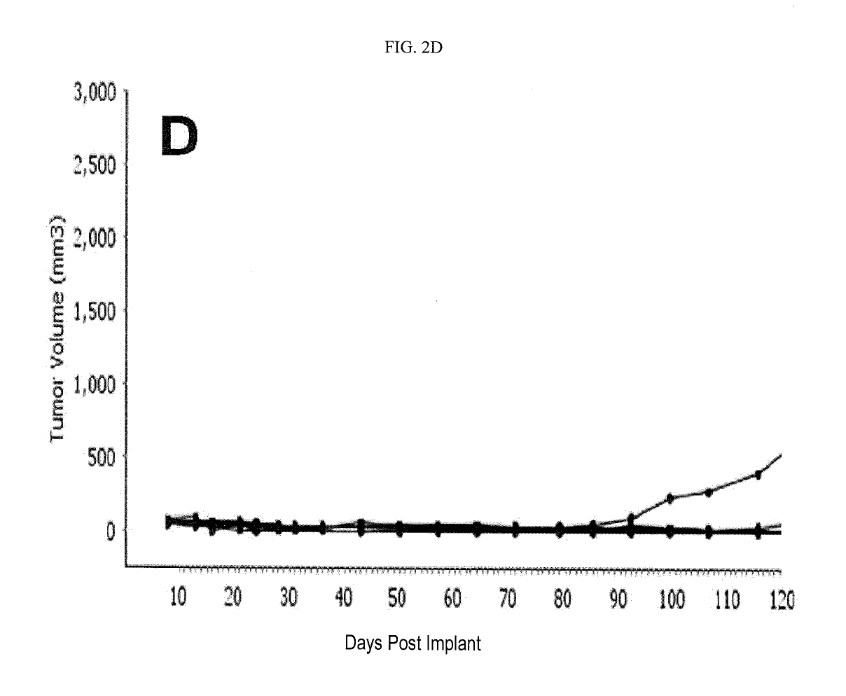

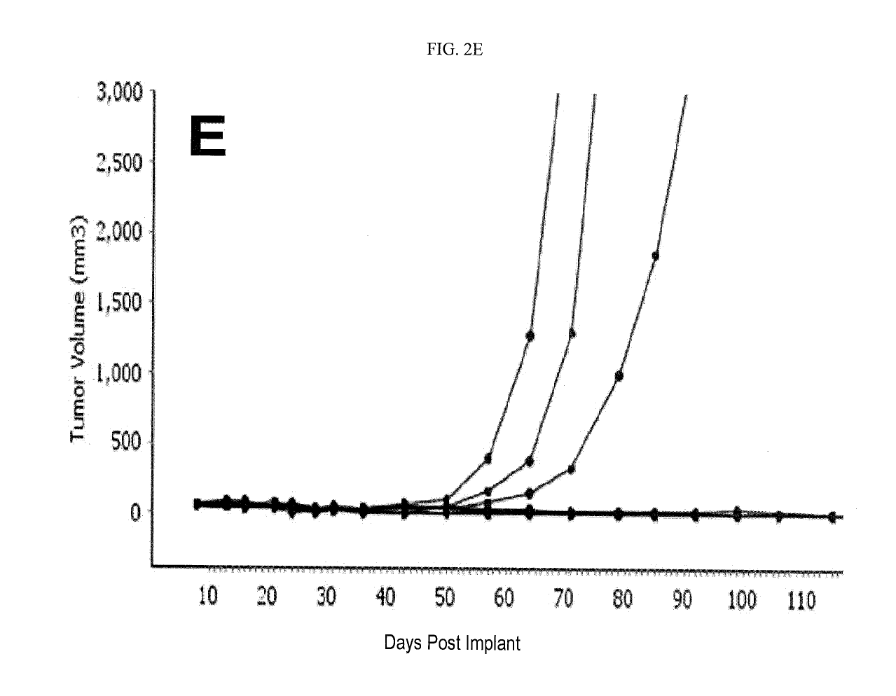

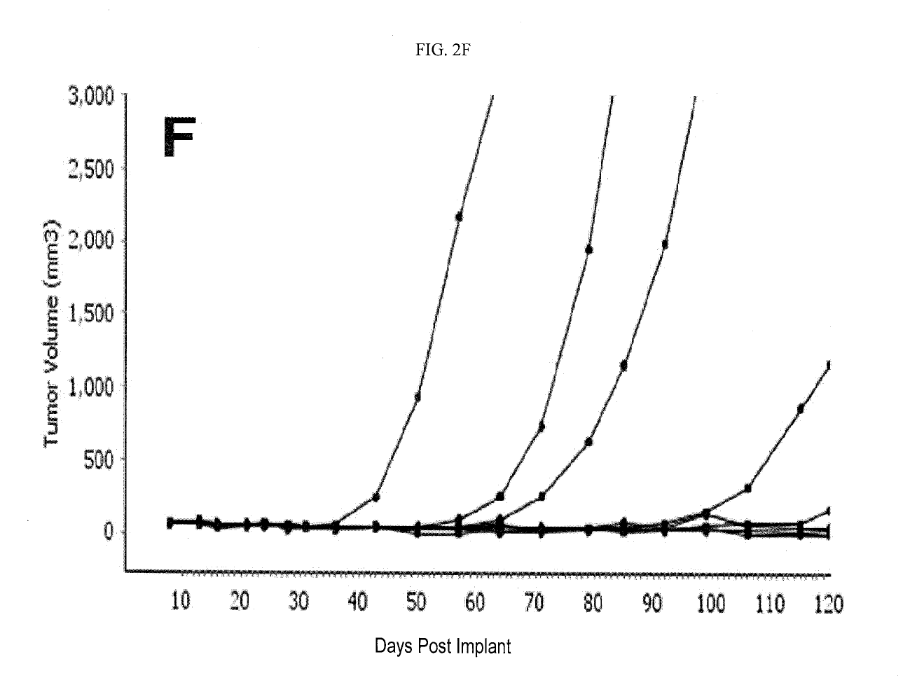

As shown in Table 1, and in FIGS. 1A-B, combination of elotuzumab and CD137 mAb resulted in higher number of mice exhibiting complete tumor responses compared to elotuzumab or CD137 mAb alone. In particular, independent of the dose of elotuzumab administered, when CD137 mAb and elotuzumab were administered on the same day (concurrent administration), complete regressions were observed in ≧50% mice (4 out of 8 mice, and 6 out of 8 mice with the combination of CD137 mAb (100 μg/mouse) plus elotuzumab at 20 μg/mouse or 100 μg/mouse respectively). In addition, greater numbers of mice with complete regressions were observed in the combination therapy groups with elotuzumab administered at the highest dose (100 μg/mouse) following any of the schedules tested. Based on these results, concurrent dosing of both therapeutic agents was selected for further studies exploring various dose levels.

TABLE 1
ANTITUMOR ACTIVITY OF ELOTUZUMAB AT TWO DOSE LEVELS
IN COMBINATION WITH CD137 MAB FOLLOWING
VARIOUS SCHEDULES

| | Dose | Schedule | Complete Regressions |
|---|---|---|---|
| Treatment | (μg/mouse) | (Study Day) | (n/Total # of Mice) |
| Control Vehicle | 0.2 mL/mouse | Day 8 | 0/8 |
| CD137 mAb | 100 | Day 8, 15, 22 | 0/8 |
| Elotuzumab | 20 | Day 8 | 0/8 |
| CD137 mAb | 100 | Day 8, 15, 22 | 4/8 |
| + | + | + | |
| Elotuzumab | 20 | Day 8 | |
| Elotuzumab | 20 | Day 8 | 1/8 |
| + | + | + | |
| CD137 mAb | 100 | Day 9, 16, 23 | |
| CD137 mAb | 100 | Day 8, 15, 22 | 2/8 |
| + | + | + | |
| Elotuzumab | 20 | Day 9 | |
| Elotuzumab | 100 | Day 8 | 1/8 |
| CD137 mAb | 100 | Day 8, 15, 22 | 6/8 |
| + | + | + | |
| Elotuzumab | 100 | Day 8 | |
| Elotuzumab | 100 | Day 8 | 5/8 |
| + | + | + | |
| CD137 mAb | 100 | Day 9, 16, 23 | |
| CD137 mAb | 100 | Day 8, 15, 22 | 3/8 |
| + | + | + | |
| Elotuzumab | 100 | Day 9 | |

### Example 2

### Methods for Assessing the Therapeutic Effect of Concurrent Administration of CD137 mAb and Elotuzumab at Various Dose Levels (1, 10, 100 μg/mouse) in the OPM-2 Multiple Myeloma Tumor Model

### Methods

Study Design: Preclinical studies were conducted to evaluate the efficacy of concurrent administration of CD137 mAb (100 μg/mouse) in combination with elotuzumab administered at various dose levels (1, 10, 100 μg/mouse) in the OPM-2 multiple myeloma tumor model. Female SCID mice (5-7 weeks old) were implanted SC with OPM-2 cells (1×10⁶ cells per mouse, mixed 1:1 with MATRIGEL®). On day 8 post-tumor cell implantation, mice were randomized into 8 experimental groups of 8 mice with a mean tumor volume of 90-95 mm³ and treatments were initiated.

### Results

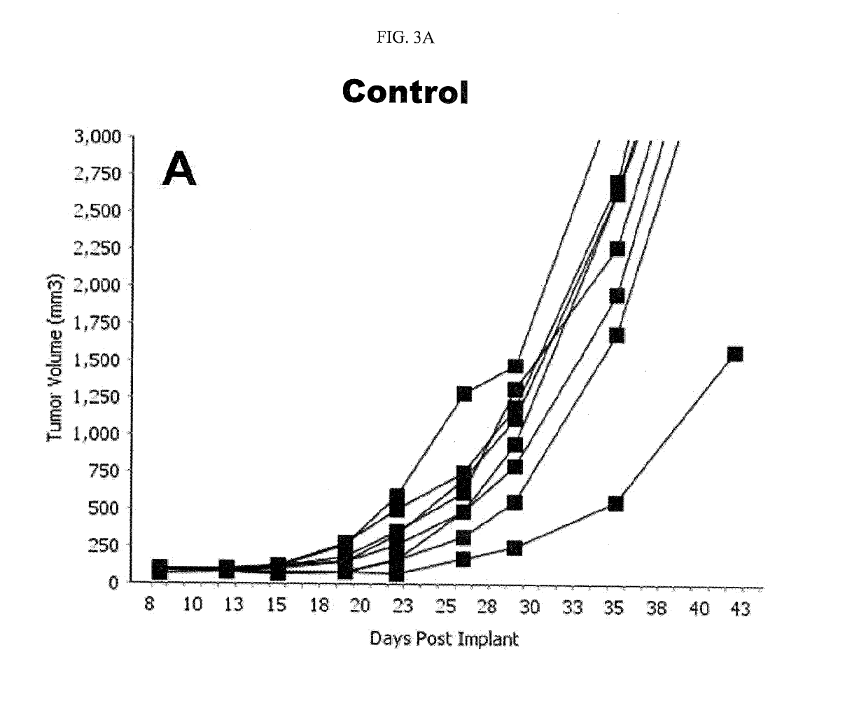

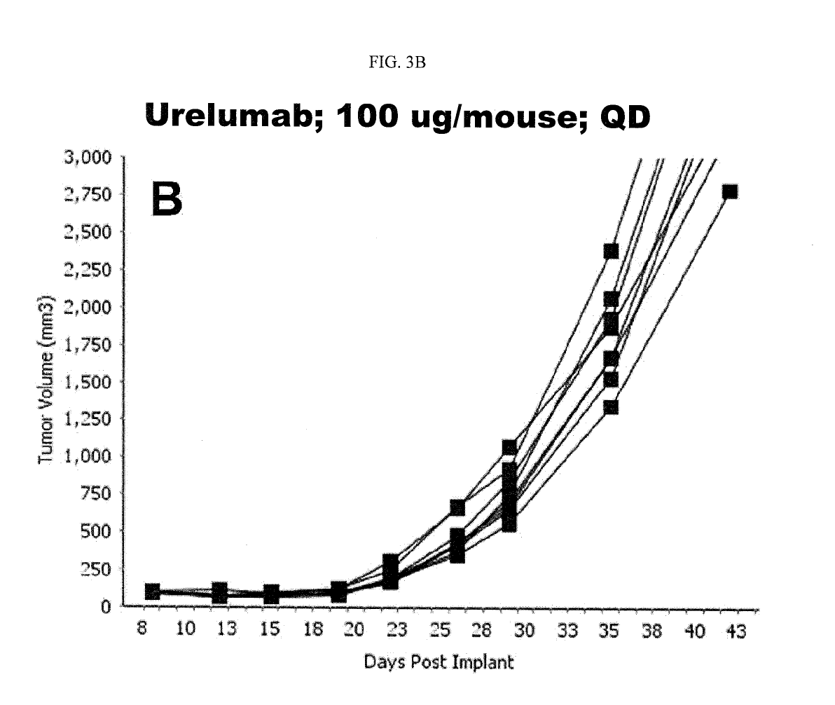

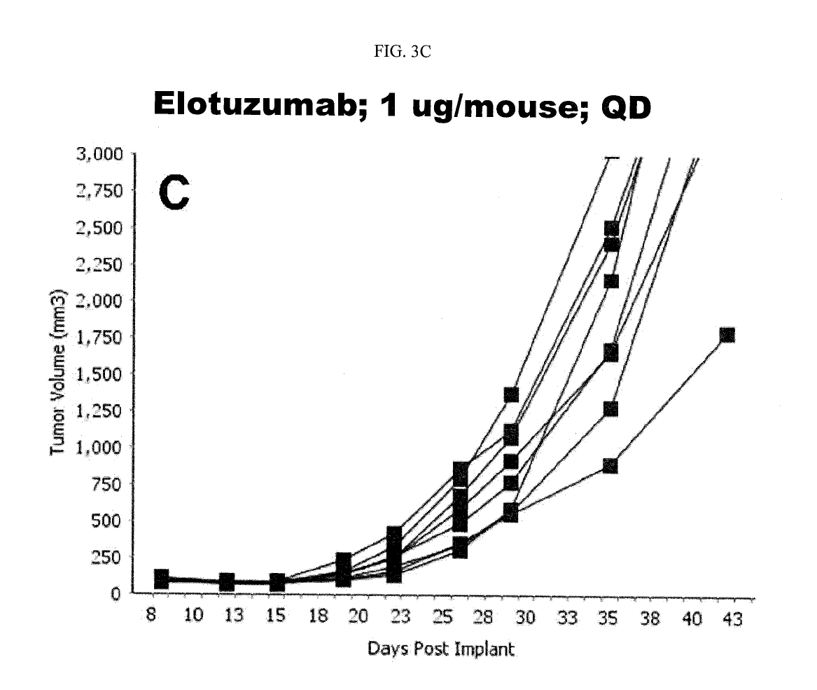

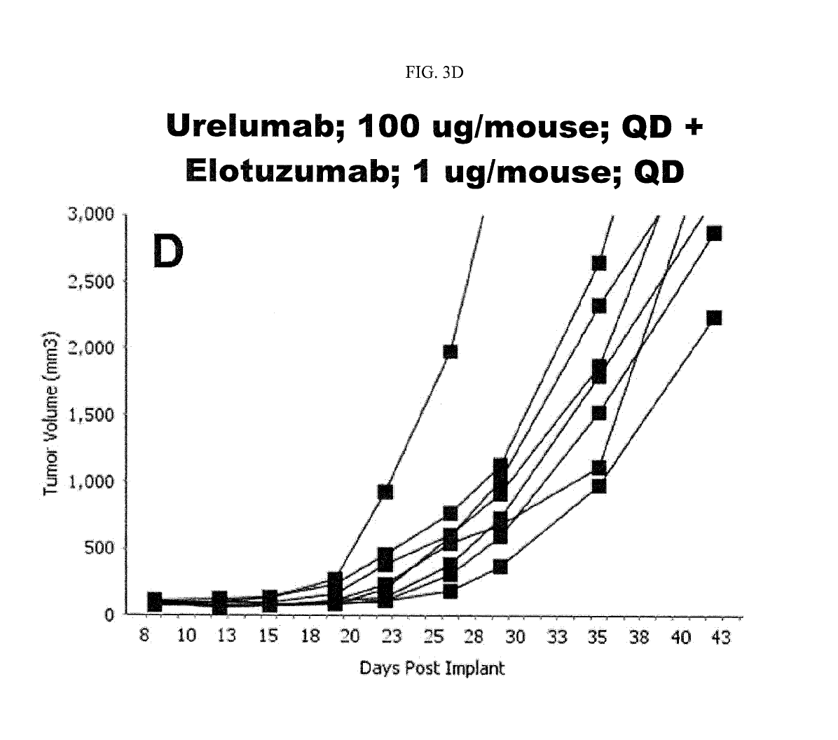

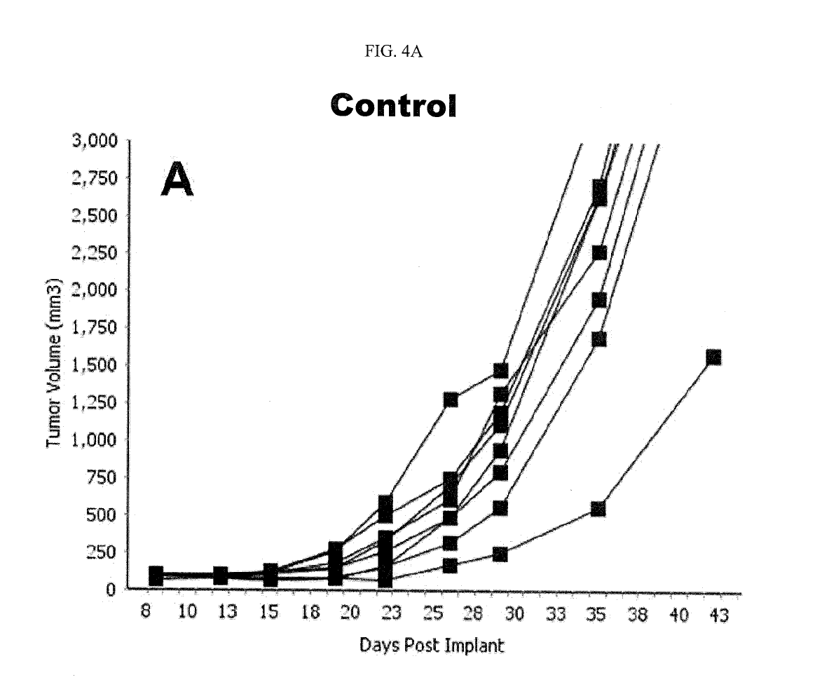

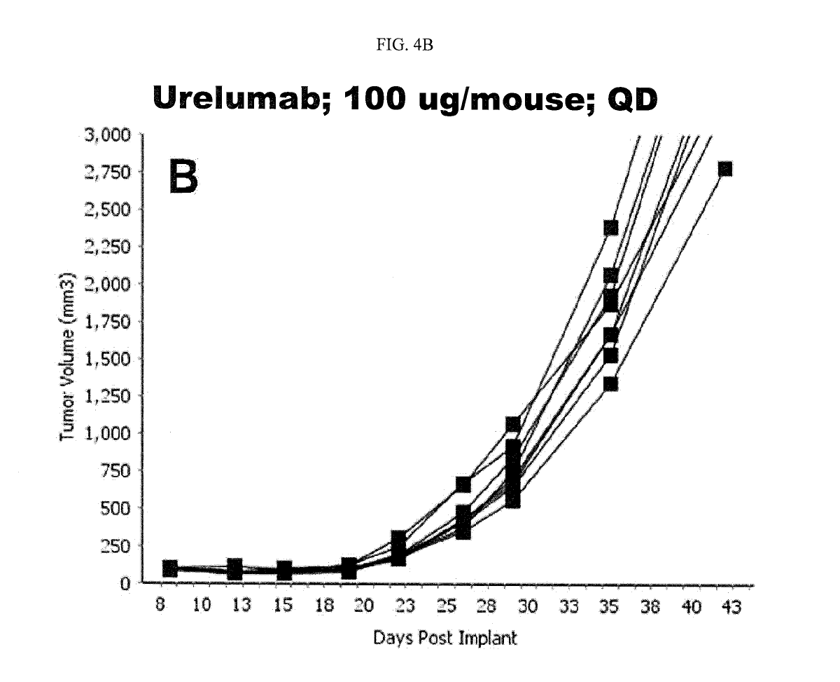

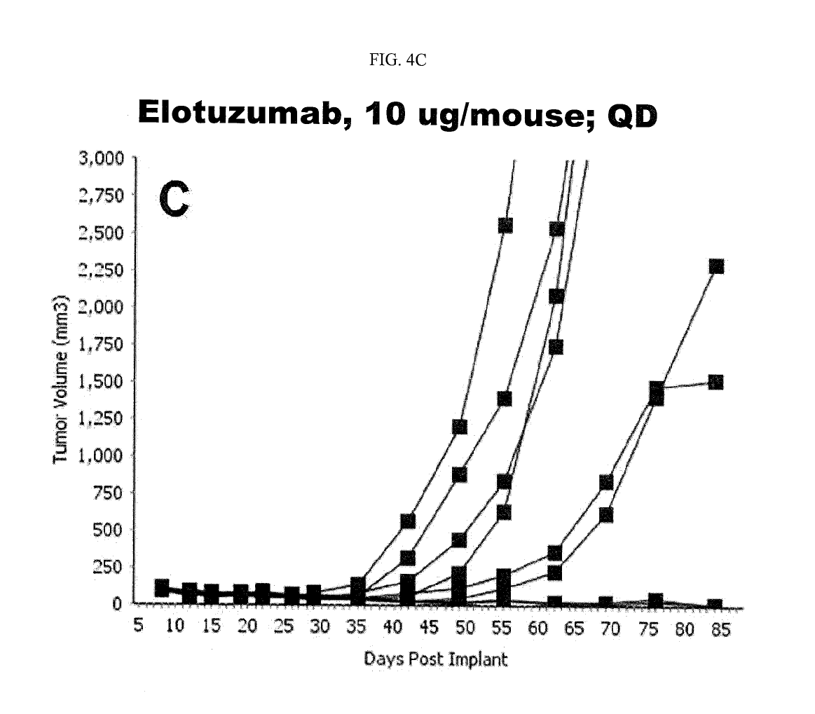

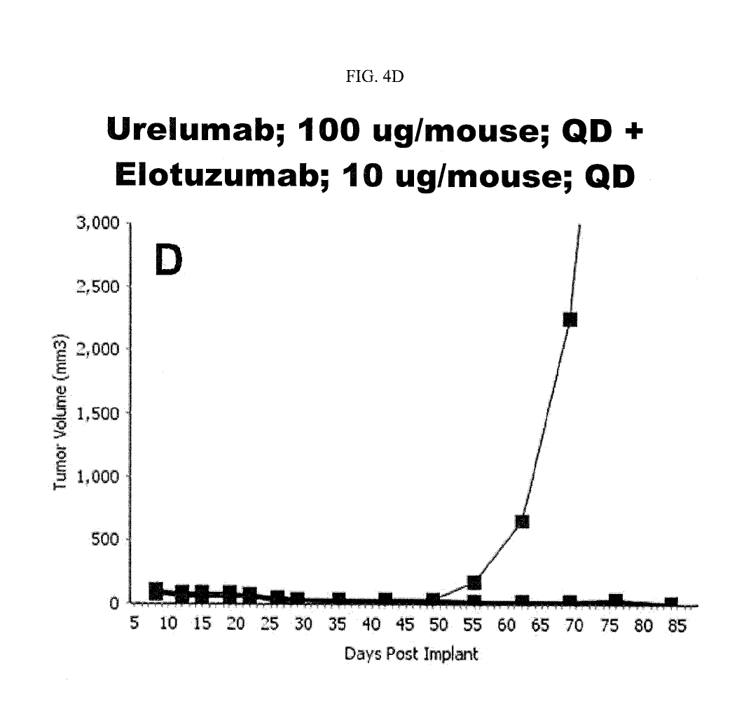

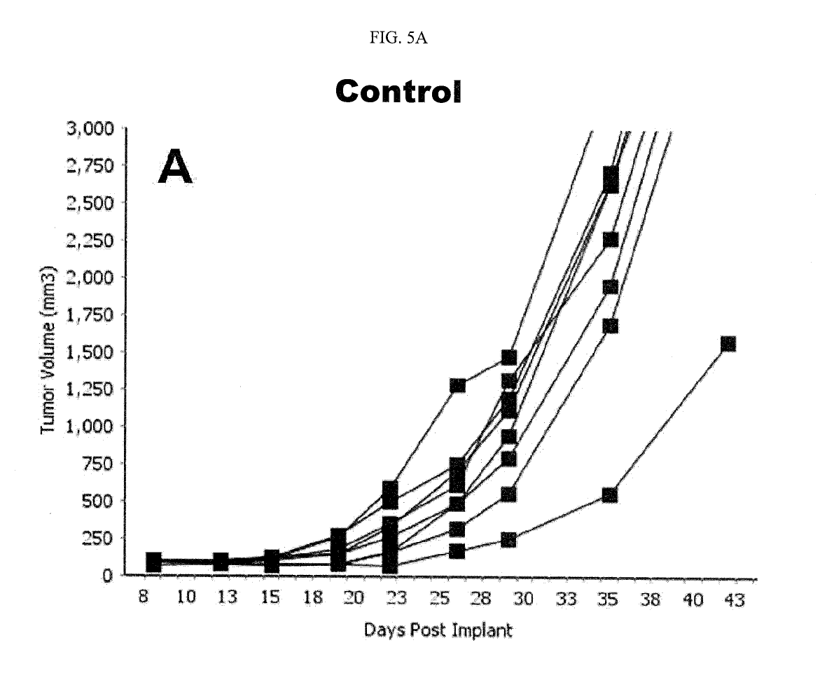

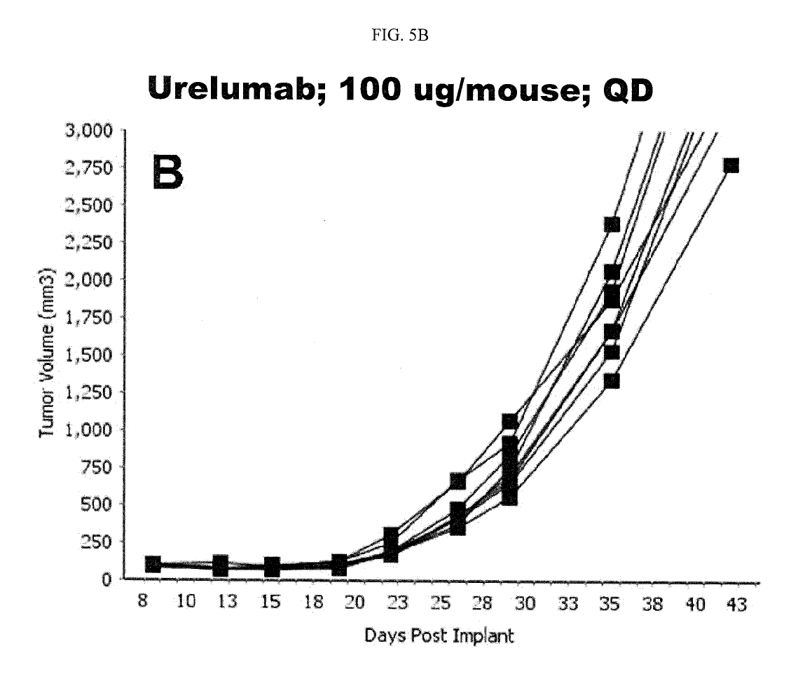

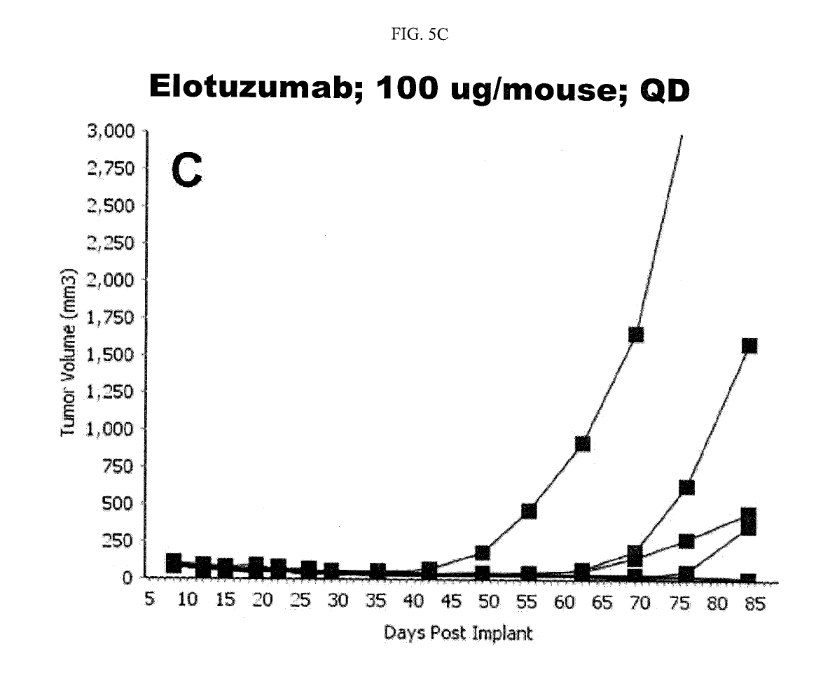

Elotuzumab as single agent demonstrated a dose-dependent effect with enhanced antitumor activity at 100 μg/mouse, while CD137 agonist antibody did not elicit significant antitumor activity. Combination therapy demonstrated greater activity with higher dose levels of elotuzumab. Marked increases in the number of mice with complete regressions were observed in the experimental groups that received CD137 mAb plus elotuzumab at 10 and 100 μg/mouse compared to elotuzumab or CD137 mAb alone as shown in Table 2, FIGS. 3A-D, 4A-D, and 5A-D.

TABLE 2
ANTITUMOR ACTIVITY OF CONCURRENT DOSING OF CD137
ANTIBODY AND ELOTUZUMAB AT VARIOUS DOSE LEVELS

| | Dose | Schedule | Complete Regressions |
|---|---|---|---|
| Treatment | (μg/mouse) | (Study Day) | (n/Total # of Mice) |
| Control Vehicle | 0.2 mL/mouse | Day 8 | |
| CD137 mAb | 100 | Day 8 | 0/8 |
| Elotuzumab | 1 | Day 8 | 0/8 |
| Elotuzumab | 10 | Day 8 | 1/8 |
| Elotuzumab | 100 | Day 8 | 2/8 |
| Elotuzumab | 1 | Day 8 | 0/8 |
| + | + | + | |
| CD137 mAb | 100 | Day 8 | |
| Elotuzumab | 10 | Day 8 | 6/8 |
| + | + | + | |
| CD137 mAb | 100 | Day 8 | |
| Elotuzumab | 100 | Day 8 | 6/8 |
| + | + | + | (2 Partial |
| CD137 mAb | 100 | Day 8 | Regressions) |

### Example 3

### Methods for Assessing the Therapeutic Effect of Concurrent Therapy with Elotuzumab in Combination with Various Dose Levels of CD137 mAb

### Methods

Study Design: Preclinical studies were conducted to study the effect of combination therapy of elotuzumab with CD137 mAb at 1, 10, and 100 μg/mouse (approximately 0.05, 0.5 and 5 mg/kg). Female SCID mice (7-8 weeks old) were implanted SC with OPM-2 cells (1×10⁶ cells per mouse, mixed 1:1 with MATRIGEL®). On day 12 post-tumor cell implantation, mice were randomized into 8 experimental groups of 8 mice with a mean tumor volume of 80-85 mm³ and treatments were initiated.

### Results

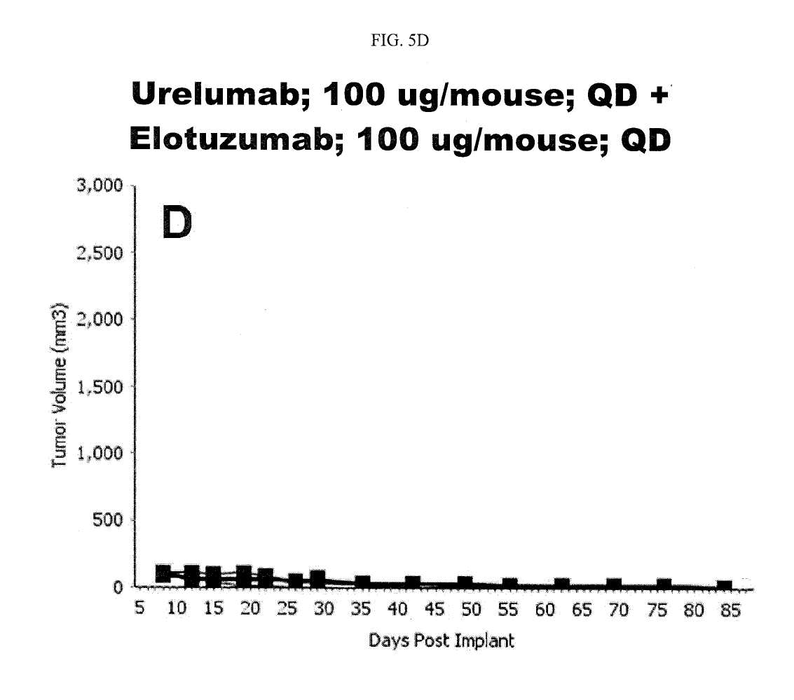

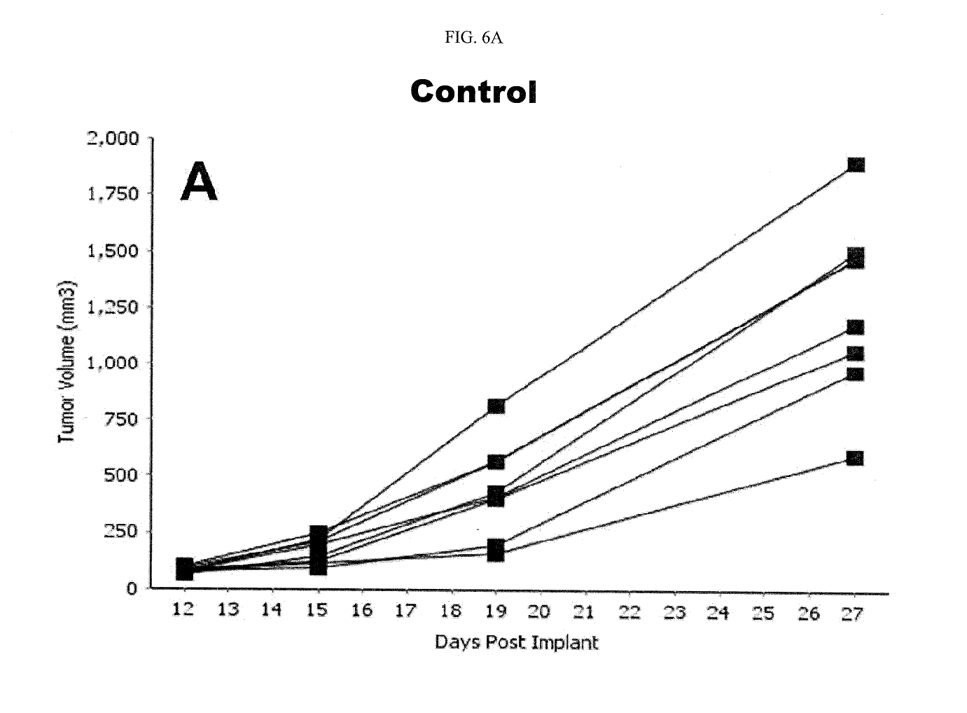

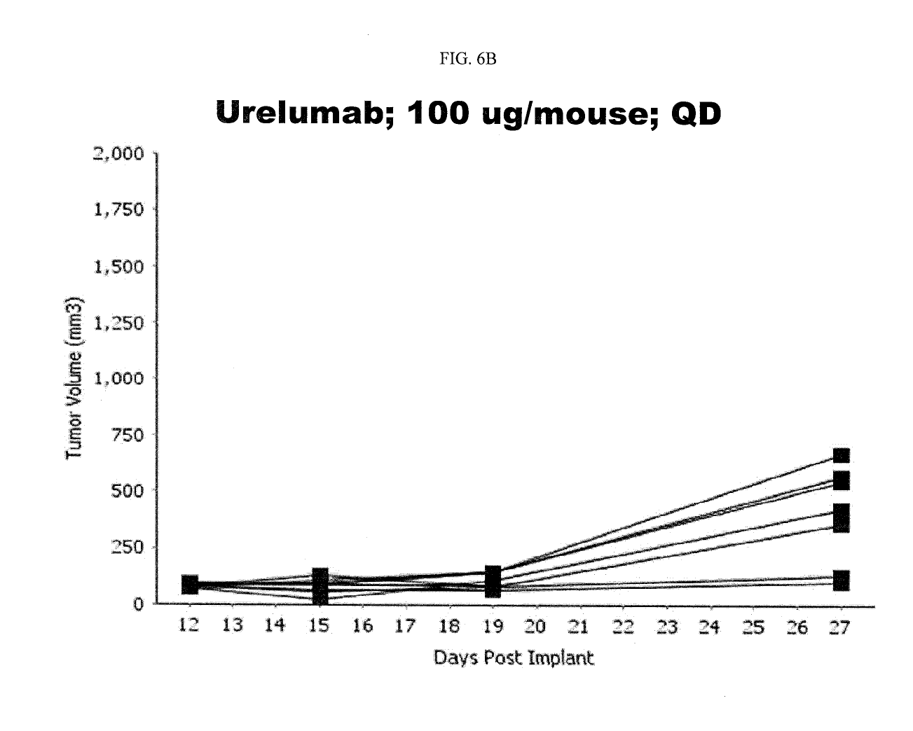

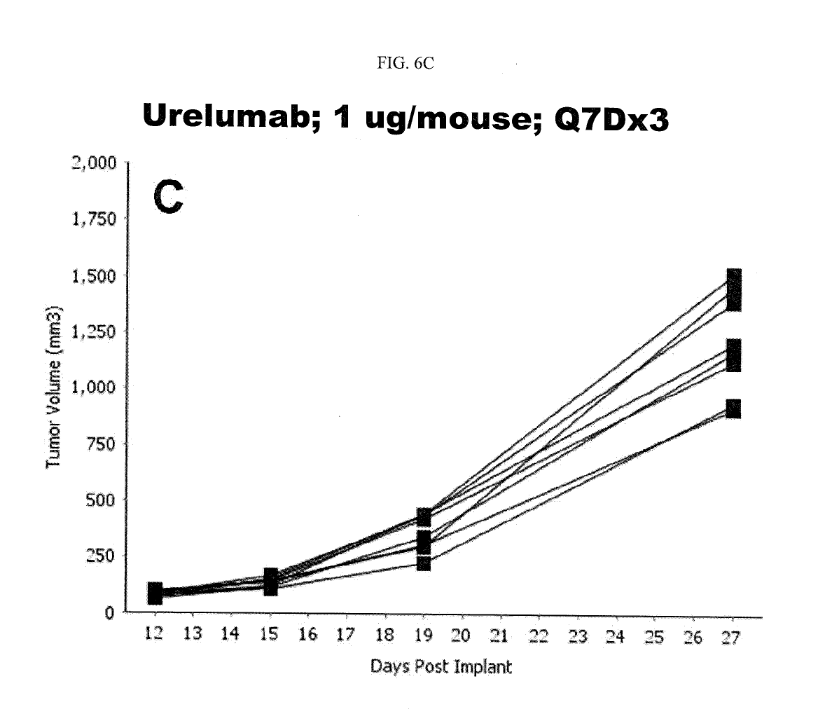

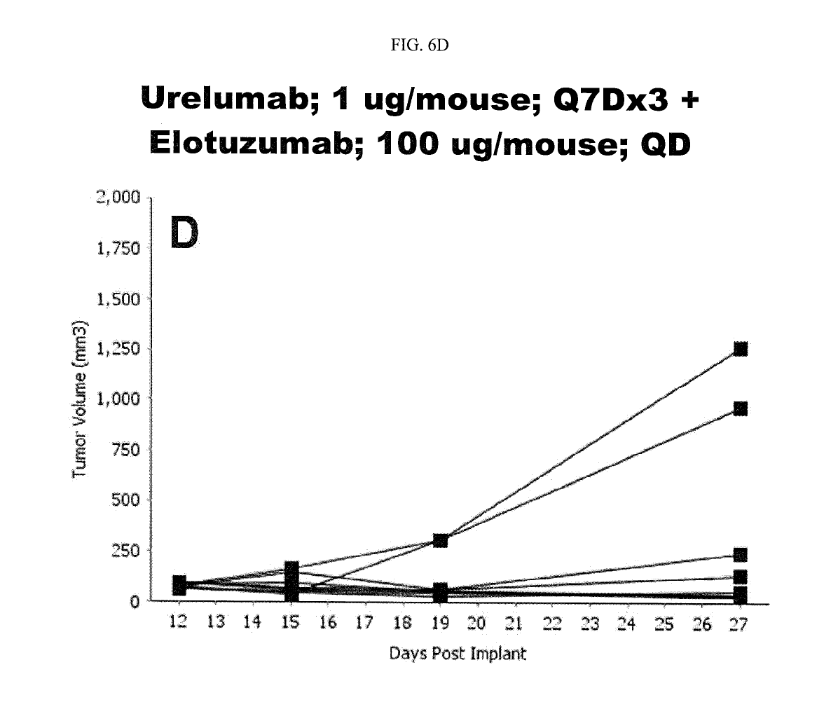

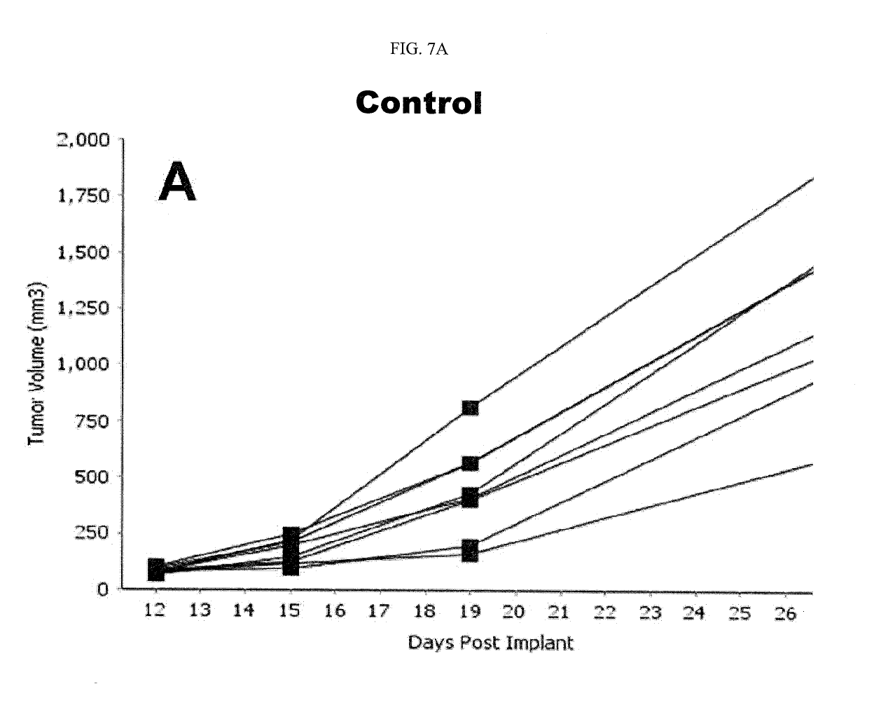

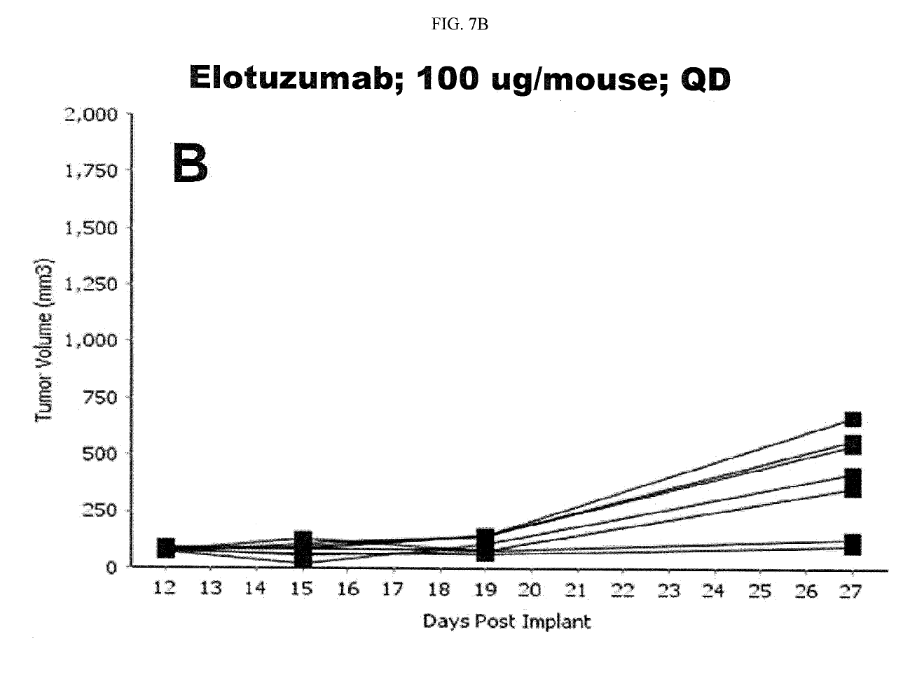

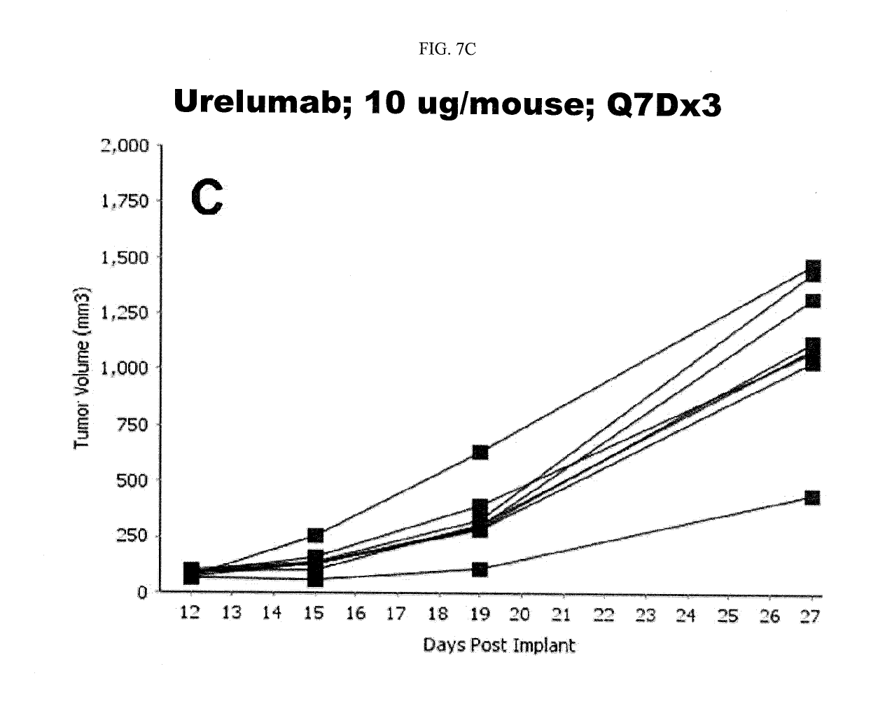

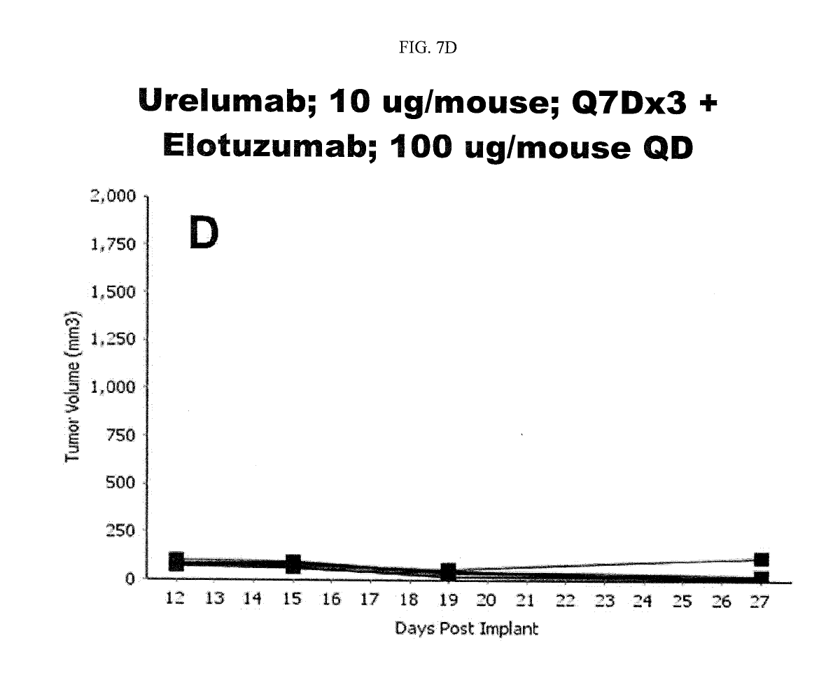

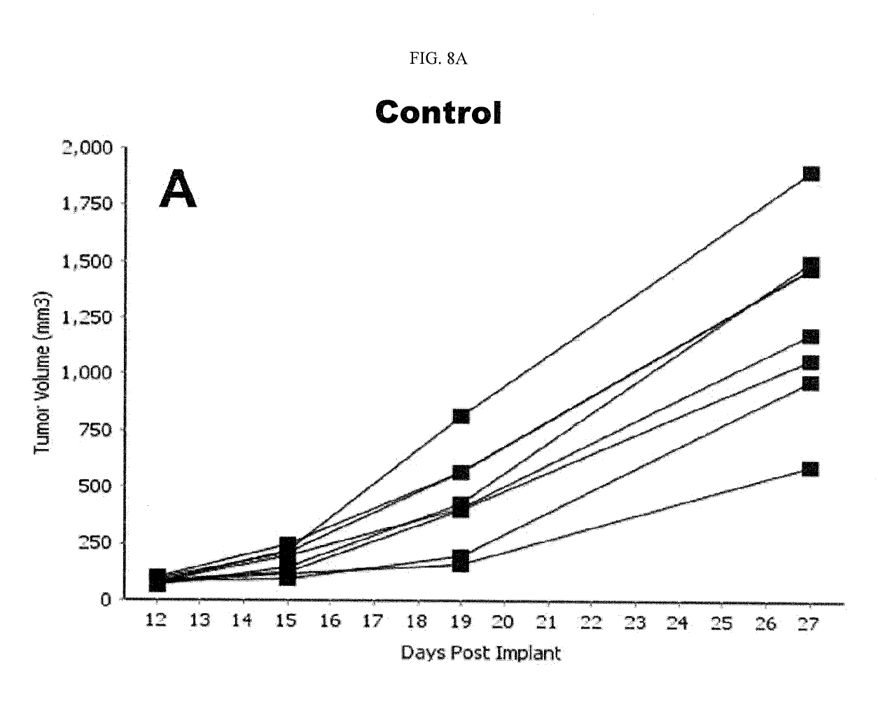

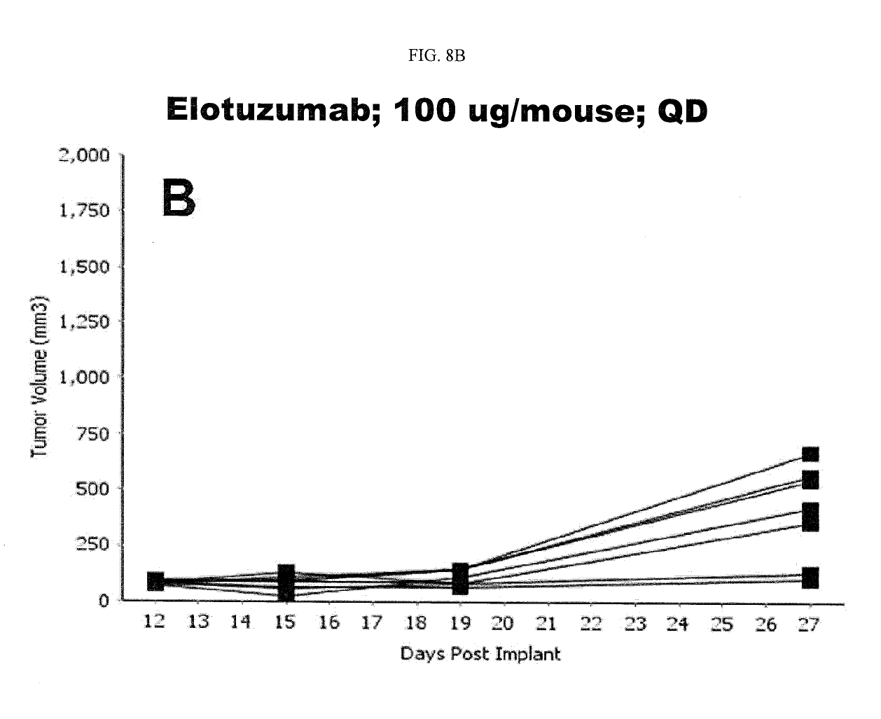

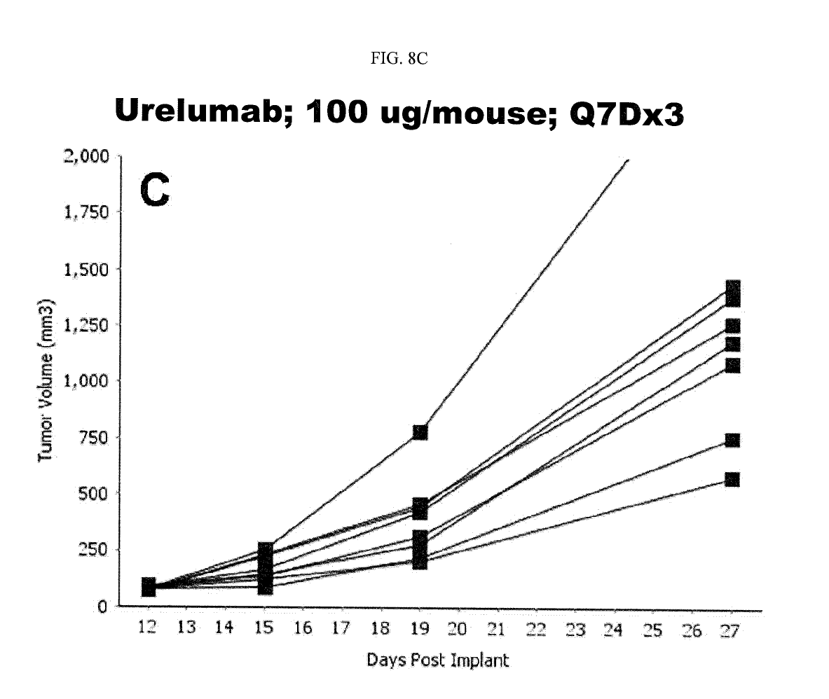

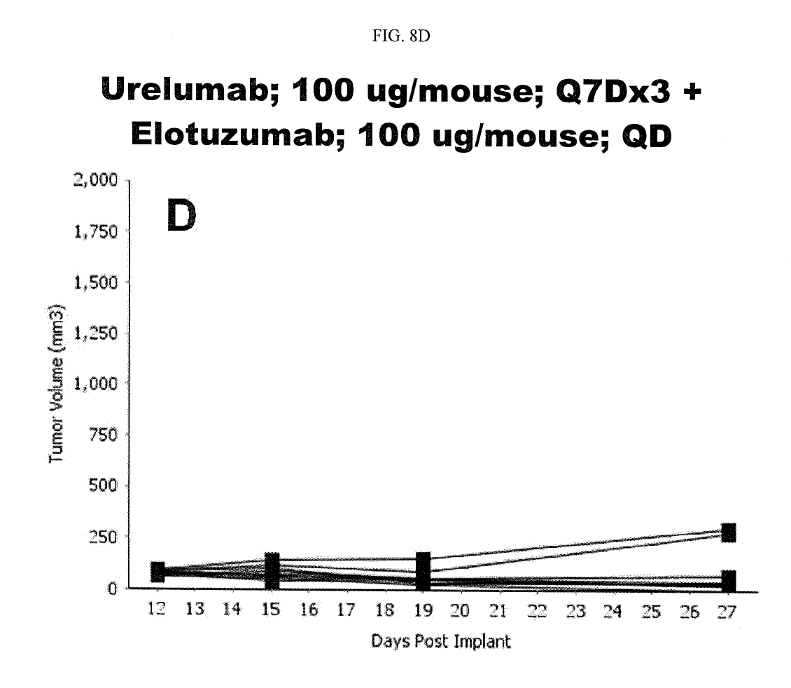

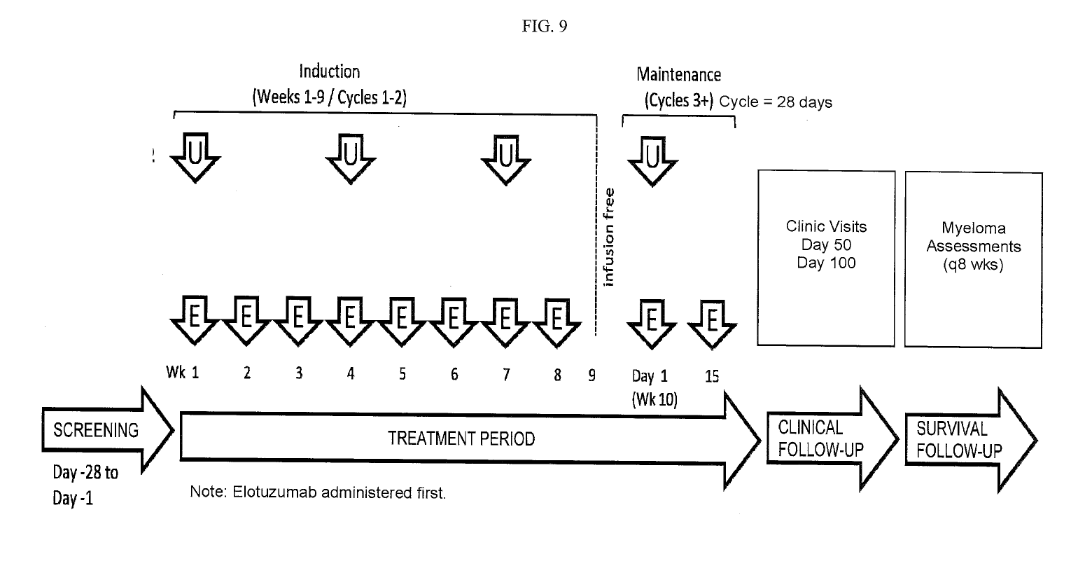

The results demonstrated that no significant antitumor effect was observed with CD137 mAb alone at any dose level. In mice treated with only elotuzumab at 100 μg/mouse, tumor growth delay was observed, but no tumor regressions (70% TGI). Conversely, a greater number of mice with complete regressions were observed in the combination groups treated with CD137 mAb at 10 and 100 μg/mouse plus elotuzumab compared to elotuzumab or CD137 mAb alone as shown in Table 3, FIGS. 6A-D, FIGS. 7A-D, and FIGS. 8A-D.

TABLE 30
ANTITUMOR ACTIVITY OF CONCURRENT THERAPY WITH
ELOTUZUMAB IN COMBINATION WITH CD137
ANTIBODY AT VARIOUS DOSE LEVELS

| | Dose | Schedule | Complete Regressions |
|---|---|---|---|
| Treatment | (μg/mouse) | (Study Day) | (n/Total # of Mice) |
| Control Vehicle | 0.2 mL/mouse | Day 8 | |
| Elotuzumab | 100 | Day 8 | 0/7 |
| CD137 mAb | 1 | Day 8 | 0/8 |
| CD137 mAb | 10 | Day 8 | 0/8 |
| CD137 mAb | 100 | Day 8 | 0/8 |
| Elotuzumab | 100 | Day 8 | 0/8 |
| + | + | + | |
| CD137 mAb | 1 | Day 8 | |
| Elotuzumab | 100 | Day 8 | 4/8 |
| + | + | + | |
| CD137 mAb | 10 | Day 8 | |
| Elotuzumab | 100 | Day 8 | 3/8 |
| + | + | + | |
| CD137 mAb | 100 | Day 8 | |

### Conclusion

In view of the foregoing results, the combination of elotuzumab with CD137 agonist antibody demonstrated synergistic results when administered concurrently, particularly at doses ranging from about 1-10 mg/kg Elotuzumab and CD137 mAb at doses ranging from about 0.5-5 mg/kg.

### Example 4

### Phase I Trial in Patients with Multiple Myeloma for Investigating Therapy with Elotuzumab in Combination with Various Dose Levels of CD137 mAb

A phase 1 trial of Agonistic CD137 (urelumab) and Anti-CS1 Antibody (elotuzumab) is conducted in patients having Multiple Myeloma to demonstrate the efficacy of administering these two therapeutics as a combination treatment.

The trial consists of two segments. Segment 1 includes dose escalation of elotuzumab in combination with urelumab in subjects with multiple myeloma. Segment 2 follows Segment 1 and includes cohort expansion of elotuzumab in combination with urelumab in subjects with relapsed/refractory multiple myeloma and subjects with post autologous transplant and have achieved very good partial response (VGPR) or complete response (CR) with minimal residual disease (MRD). In both segments, subjects receive elotuzumab and urelumab in two stages (Induction and Maintenance). During Induction, subjects are administered intravenous (IV) doses of elotuzumab weekly for 8 doses and IV doses of urelumab every 3 weeks for 3 doses. During Maintenance, subjects are administered IV doses of elotuzumab every 2 weeks and urelumab every 4 weeks, for up to 26 weeks of study therapy.

Subjects will be assigned to Segment 1 or Segment 2 for both dose escalation and cohort expansion. In cohort expansion, the allocation of subjects will be performed in a randomized manner within the disease populations of interest, to be defined as Disease Groups A or B. Disease Group A will consist of subjects with relapsed/refractory multiple myeloma, and Disease Group B will consist of subjects who are post autologous transplant and have achieved very good partial response (VGPR) or complete response (CR) with minimal residual disease (MRD) detected by multiparameter flow cytometry (MFC). If either Treatment is not enrolling (i.e., DLT assessment period or terminated), eligible subjects will be allocated into the enrolling Treatment Arm.

### 1. Objectives

The primary objective of this study is to assess the safety and tolerability of elotuzumab administered in combination with urelumab and to identify dose limiting toxicities (DLTs) and the maximally tolerated dose (MTD) of the combination, in subjects with multiple myeloma.

Secondary objectives include assessing the preliminary anti-tumor activity of the combination, characterizing the pharmacokinetics (PK) of the combination, monitoring immunogenicity of the combination, and assessing the pharmacodynamic effects of the combination on cell number and function of bone marrow plasma cells and natural killer cells in each disease group.

Exploratory Objectives include assessing the pharmacodynamic effects of the combination on peripheral natural killer and T cell function and phenotype, explore the association of plasma cells and NK and T cell phenotype and clinical outcome, exploring the relationship of safety and efficacy with changes in plasma cells and natural killer and T cell function, assess treatment dependent changes in NK and T cell function, assess treatment dependent changes in soluble cytokines and chemokines, and assessing the landmark overall survival at three years following the start of therapy with the combination.

### 2. Overall Objectives

This is a randomized phase I, open label study that will enroll the following select subjects with multiple myeloma: subjects with relapsed and or refractory disease, subjects who are post autologous transplant and have achieved very good partial response (VGPR) or complete response (CR). This study is performed in two segments: dose escalation and cohort expansion. Dose escalation is performed to characterize the safety and tolerability of elotuzumab administered in combination with urelumab in subjects with multiple myeloma, and is followed by a cohort expansion in the distinct disease groups: Group A or B. Cohort expansion groups establish expanded safety experience with the combination and enable characterization of the immunoregulatory (biomarker) activity and preliminary antitumor efficacy of elotuzumab with urelumab. Study treatment in both segments is divided into two distinct parts: Induction and Maintenance.

In both segments, subjects complete up to four periods of the study: Screening (up to 28 days), Treatment (Induction and Maintenance), and Clinical Follow-up (100 days). Myeloma disease assessments may continue beyond this period for subject with clinical benefit, as specified for the Response Follow-up period.

Study Treatment—Induction Phase (Week 1 Through Week 9):

Subjects receive intravenous (IV) doses of elotuzumab weekly for 8 doses and urelumab every 3 weeks for 3 doses. Week 9 is an infusion-free week.

Study Treatment—Maintenance Phase:

Subjects receive IV doses of elotuzumab every 2 weeks and urelumab every 4 weeks beginning at week 10, for up to 26 weeks of study therapy (additional study treatment beyond 26 weeks will be assessed on a case-by-case basis upon risk-benefit ratio).

Subjects in the cohort expansion segment are treated at the maximally tolerated dose (MTD), the maximally administered dose (MAD), or at an alternative dose.

The decision to treat a subject with additional cycles of study therapy is based on disease assessment. Subjects with an overall response of CR unconfirmed, PR, SD, or PD-unconfirmed continue therapy until they develop PD-confirmed, CR-confirmed, experience clinical deterioration, develop adverse events requiring discontinuation, withdraw consent, or complete both Induction and Maintenance.

The decision to treat a subject with additional cycles of study therapy will be based on disease assessment. Treatment decisions related to subject management will be based on International Myeloma Working Group (IMWG) criteria (see Appendix 1 for definitions) and MRD detection by multiparameter flow cytometry (MFC) for Group B in cohort expansion. Subjects with an overall response of CR unconfirmed, VGPR, PR, SD, PD-unconfirmed or MRD positive (Group B in cohort expansion) will continue therapy until they develop CR-confirmed, stringent CR (sCR), PD-confirmed, MRD negative (Group B in expansion cohort) confirmed, experience clinical deterioration, develop adverse events requiring discontinuation, withdraw consent, or maximum of 26 weeks of treatment.

Subjects who: (1) have CR, sCR, MRD negative (Group B in expansion cohort)-confirmed, (2) complete Induction and Maintenance (26 weeks) or (3) develop toxicity requiring discontinuation of the study therapies will enter the Clinical Follow-up period to evaluate for any new adverse event with Follow-up visits at 50 and 100 days after the end of treatment visits. These subjects will be followed in the Response Follow-up period. These subjects will undergo multiple myeloma disease assessments (per IMWG criteria) every 8 weeks after stopping study drug, until progression, starting a new treatment, lost to follow up, or death, whichever comes first.

Subjects completing 26 weeks of treatment with ongoing disease control (CR, sCR, MRD negative, VGPR, PR or SD) may be eligible for continued treatment after carefully evaluated by the BMS Medical Monitor on a case-by-case basis to determine whether the risk/benefit ratio supports administration of further study therapy.

Retreatment: Subjects completing 26 weeks of treatment and entering Follow-up Period, with ongoing disease control (CR, sCR, MRD negative, VGPR, PR or SD) with documentation of subsequent confirmed disease progression within 12 months of the last dose of study drug may be eligible for retreatment. Each subject's eligibility for retreatment will be carefully evaluated by the BMS Medical Monitor on a case-by-case basis to determine whether the risk/benefit ratio supports administration of further study therapy. Subjects meeting criteria for retreatment may be treated up to an additional 26 weeks at the same dose and schedule administrated or the next lower dose if the original dose and schedule were determined to exceed the MTD. Subjects who have PD-confirmed on study therapy will enter Clinical Follow-up to continue monitoring for adverse events.

At each Clinical Follow-up visit, assessments will include physical examinations, adverse event assessment, safety laboratory testing, and disease assessment. If an adverse event has not resolved by the end of the Clinical Follow-up period, the subject may continue follow-up until the AE has resolved to grade≦1 or baseline, or is deemed irreversible by the investigator.

### 3. Dose Escalation

A total number of 6 or 9 subjects will be treated during the dose escalation phase of elotuzumab given in combination with urelumab for a given dose level. The Dose Limiting Toxicity (DLT) observation period will last 6 weeks (42 days) from initiation of study therapy. Although safety data from at least 6 subjects at a given dose level of elotuzumab given in combination with urelumab would be needed for safety assessment, the eligible multiple myeloma subjects will be enrolled in increments of 3 (up to total of 9) to avoid exposure to 6 subjects at once. Initially 3 eligible multiple myeloma subjects will be treated at 10 mg/kg elotuzumab in combination with 8 mg of urelumab. Up to 9 subjects, in increments of 3, may be added to the same dose level, and hence a decision to stay at the same dose level to expand to next phase, or to consider the next lower dose level (dose −1; 10 mg/kg elotuzumab in combination with 3 mg urelumab), will be guided by the number of subjects with DLTs observed during the dose escalation phase (see Table 4 below).

A dose level of 10 mg/kg of elotuzumab in combination with 3 mg of urelumab (Dose −1) may be considered if the safety and tolerability profile for 10 mg/kg elotuzumab in combination with 8 mg urelumab is evaluated as not acceptable, after consultation and agreement between the Investigator(s) and the sponsor as well as review of the existing clinical safety database from earlier studies. Following a similar procedure, if the dose level of 3 mg urelumab in combination with Elotuzumab is evaluated as not acceptable as well, the findings will be discussed between the Investigator(s) and the Sponsor and an agreement will be reached as to whether a lower dose of urelumab should be examined.

No intra-subject dose escalation or reduction is allowed. Subjects who withdraw from the study during the DLT period for reasons other than toxicity may be replaced within the same dose cohort. Subjects in dose escalation will be monitored continuously beyond the DLT period as well, to evaluate safety beyond the DLT period.

All available clinical and laboratory data, and the nature, time of onset, and time to resolution of DLTs observed during dose escalation will be reviewed to determine whether an alternate dose schedule should be examined in consultation between the investigators and the Sponsor, if needed. If agreed upon, the alternate schedule will be identified by a protocol amendment. If the MTD is exceeded in the first cohort, the evaluation of alternate doses and schedules of urelumab may be investigated. The dose selection table is provided below:

TABLE 4
SCHEDULE OF PLANNED DOSE ESCALATION

| | Urelumab | Elotuzumab | Number of |
|---|---|---|---|
| Dose Cohort | mg | mg/kg | Subjects |
| 1 | 8 | 10 | approximately 6-9 |
| −1 | 3 | 10 | approximately 6-9 |

### 4. Cohort Expansion

Cohort expansion is initiated at the MTD, the maximum administered dose (MAD), or an alternate dose, if recommended. Subjects are randomized to receive elotuzumab with urelumab. Enrollment is limited to one of three specified patient populations with multiple myeloma; Treatment Group A) subjects with relapsed and/or refractory disease, Treatment Group B) subjects who are post an autologous transplant and have achieved VGPR or CR with MRD detected by multiparameter flow cytometry.

The sample size for each arm will be guided by Gehan design (Therapeutics, 13(4):346-353 (1961)). In order to determine whether a 25% response is likely, 9 subjects will be treated at first (Stage I) in each of the two disease groups. In a disease group within each arm for which no response is observed, it will be concluded that the true response rate is unlikely to be greater than or equal to 25%, and no more subjects will be enrolled. Otherwise, in a group for which at least one response among the first 9 patients is observed, between 0 and 16 additional subjects will be treated for a total of 9 to 25 per group. At Stage 1, approximately 18 subjects will be randomly assigned to (9 subjects) in Groups A and B. The enrollment of subjects in Stage II would be guided by the number of responders observed in Stage 1.

Treatment Groups A and B:

At Stage I, 18 subjects in each group will randomly be assigned to receive study drug in Induction to be followed by Maintenance. Additionally, all subjects will be required to undergo bone marrow aspirate and biopsy prior to the initiation of study therapy (Screening), and at designated time points.

Clinical safety monitoring of subjects enrolled during the cohort expansion segment of the study is identical to that conducted during the dose escalation segment of the study. As enrollment proceeds during cohort expansion, if the combined incidence of study drug related DLTs requiring dose modification exceeds 33% of treated subjects, further enrollment to that cohort is interrupted and the findings are be discussed. An agreement is reached whether a lower dose or an alternate dose or dose schedule of the combination is examined, or whether any additional treatment guidelines are to be implemented prior to enrollment of additional subjects.

### 5. Dose Limiting Toxicity

For the purpose of guiding dose escalation decision making, DLTs are determined based on the incidence and severity of study drug-related adverse events (AE) occurring within 6 weeks (42 days) of initiation of study therapy. Adverse events are graded according to the National Cancer Institute (NCI) Common Terminology Criteria for Adverse Events version 4.0 (CTCAEv4). For the purposes of subject management, DLTs generally lead to dose interruptions regardless of the cycle in which a DLT occurs.

### 6. Duration of Study

The Screening Period will last up to 28 days. The Treatment Period (Induction and Maintenance) will last up to 26 weeks for Arm 2. The Clinical Follow-up Period will last 100 days. The Response Follow-up Period will consist of myeloma assessments every 8 weeks or until disease progression, start of new treatment, lost to follow-up, or death, whichever comes first. The total time on study for any individual subject is estimated to be approximately 3 years. The total duration of the study is estimated to be 3.5 years from the time of the first visit of the first subject to the required follow-up of the last subject enrolled. The approximate number of subjects dosed for the entire study will be 48 to 136 subjects (approximately 12 to 36 subjects during dose escalation and approximately 36 to 100 subjects during cohort expansion).

### 7. Study Population

Female and male subjects, ages 18 years or older who meet all eligibility criteria will be eligible to participate in the study. Subjects must have histological confirmation of multiple myeloma with measurable disease (per IMWG criteria). Part 1 (dose escalation) will include subjects with relapsed/refractory multiple myeloma and Part 2 (cohort expansion) will include relapsed/refractory or post autologous transplant and have achieved very good partial response (VGPR) or complete response (CR) with minimal residual disease (MRD) detected by multiparameter flow cytometry (MFC).

### 8. Study Assessments

### Safety Outcome Measures:

Adverse events will be assessed continuously during the study and for 100 days after the last treatment. Adverse events will be coded using the most current version of MedDRA and reviewed for potential significance and importance. Adverse events will be evaluated according to the NCI CTCAE Version 4.0. Subjects should be followed until all treatment related adverse events have recovered to grade≦1 or baseline, or are deemed irreversible by the investigator. Safety assessments will be based on medical review of adverse event reports and the results of vital sign measurements, ECGs, physical examinations, and clinical laboratory tests.

### Efficacy Assessments:

Disease assessment with serum and urine myeloma lab tests, bone marrow assessment and computed tomography (CT) and/or magnetic resonance imaging (MRI), as appropriate, will be performed at baseline. From the start of maintenance, serum and urine myeloma lab tests will be performed at week 10 and every 8 weeks prior to dose administration. For subjects in expansion Group B (post autologous transplant), additional bone marrow aspirate for MRD assessment by multiparameter flow cytometry will be performed at screening, cycle 4 (week 14), cycle 7 (week 26) and then every 6 cycles thereafter until disease progression. CT or MRI or both will be performed, if appropriate, every 12 weeks for the assessment of plasmacytomas. Disease assessments will continue until there is confirmed disease progression, at the completion of follow-up, or until subjects withdraw from the study. Myeloma responses will be based on investigator assessment and determined as defined by IMWG criteria (see Appendix 1 and Appendix 3) and MRD detection by multiparameter flow cytometry (Group B in expansion cohort). In the absence of clinical deterioration, any initial assessment of progressive disease (PD) complete response (CR) stringent complete response (sCR) or MRD negative by MRD (Group B) will be confirmed by a repeat evaluation no sooner than 4 weeks later.

### Pharmacokinetic Measures:

Pharmacokinetic parameters (Cmax, Cmin, Tmax, AUC(INF), AUC(TAU), T-HALF, % UR, CLT/F, CLR, Vss, and AI) are derived from plasma concentration versus time and urinary excretion data.

Bone Marrow aspirates (tumor biopsies) are obtained from a minimum of twelve subjects in the smoldering myeloma expansion cohorts at baseline and post-treatment times. All subjects in cohort expansion are offered the opportunity of undergoing biopsies. All subjects who undergo biopsies are required to have peripheral blood collected in parallel for comparison of effects on bone marrow and peripheral immune and tumor cells.

### 9. Statistical Considerations

### Dose Escalation:

The same size at each dose depends on observed toxicity and cannot be precisely determined. There will be 6 to 18 subjects in each cohort.

### Cohort Expansion:

In cohort expansion, the sample size for each arm and disease group will be guided by a 2-Stage Gehan design (Therapeutics, 13(4):346-353 (1961)). In order to determine whether a 25% response rate or MRD conversion rate is likely, 9 subjects will be treated at Stage 1 in each of the disease groups. For an arm/disease group(s) in which no responses are observed, it will be concluded that the true response rate is unlikely to be greater than or equal to 25% and no more subjects will be enrolled in these group(s). Otherwise, for group(s) in which at least one response among the first 9 patients is observed, up to an additional 16 subjects will be enrolled. With 9 subjects in Stage I, per group there is no more than 10% chance of declaring that there is no therapeutic effect when actually there is an effect of at least 25%. The above numbers are approximate, as subjects who had a response during dose escalation in a group under the same setting (e.g., dose) as in dose expansion may be included in the total n per disease group. A total of up to 25 (9+16) subjects across the 2 stages per arm/disease group, would provide an estimate of the true response rate with a standard error of approximately 10%. Overall, the two stage design will control the probability of rejecting an effective drug (to ≦10%), allows an early decision to stop treatment in a group in which a response in at least 25% of patients would be unlikely, and provides good precision around estimates of response rate or MRD conversion rates

### 10. Endpoints

The primary endpoint of this phase 1 study is safety as measured by the rate of adverse events (AEs), serious, adverse events (SAEs), deaths, and clinically significant laboratory abnormalities. Safety is evaluated on treatment, and for up to 100 days after the last dose of study drug is received. All subjects who receive any urelumab or elotuzimab are included in the safety analyses.

Secondary efficacy endpoints vary by disease state. The objective response rate is determined based on investigator assessment per the modified IMWG criteria and MRD detection by multiparameter flow cytometry (Group B in expansion cohort): Anti-tumor activity in both disease groups will be measured by the following end points: Best Overall Response (BOR), Objective Response Rate (ORR), median Duration of Response (mDOR), median Time to Response (mTTR) and progression free survival rate (PFSR) and M-protein levels.

The first secondary objective relates to anti-tumor activity and will be measured by the following secondary endpoints: in both Groups A and B:
Best Overall Response (BOR) is the best response designation over the study as a whole, recorded between the date of first dose of study medication and the date of objectively documented progression per the disease specific criteria (see appendix 3) or subsequent anti-cancer therapy, whichever occurs first, in the study. sCR, CR, VGPR or PR determinations included in the BOR assessment must be confirmed by a consecutive second (confirmatory) evaluation meeting the criteria for response that is performed at least 4 weeks after the criteria for response are first met.
Objective Response Rate (ORR): The total number of subjects whose best overall response (BOR) is either a sCR, CR, VGPR, or PR divided by the total number of subjects in the population of interest.
Median Duration of Response (mDOR): The significance of ORR is assessed by its magnitude and duration of response. DOR for a subject with confirmed response is defined as the time from first response (sCR, CR, VGPR or PR) to the date of the first documented disease progression as determined by disease specific criteria (Appendix 3) or death due to any cause, whichever occurs first. Subjects who remain alive and have not progressed will be censored on the date of their last tumor assessment (prior to subsequent cancer therapy). Response duration will only be evaluated in subjects with objective response of sCR, CR, VGPR or PR.
Median Time to Response (mTTR): Time to response (TTR) for a subject is defined as the time from date of first dose of study medication to the date of the first documented objective response (sCR, CR, VGPR or PR). TTR will only be evaluated in subjects with objective response of sCR, CR, VGPR or PR.
Progression Free Survival Rate (PFSR): The proportion of subjects remaining progression free and surviving to pre-specified time points (e.g., 24 weeks, 48 weeks, 96 weeks). This proportion will be calculated. by the product-limit method (Kaplan-Meier estimate) which takes into account censored data
M-protein levels: In both Groups A and B, the change from baseline in M-protein levels over time will be reported based on measurements at week 10 and every 8 weeks thereafter, until the subject is off study. In addition to above endpoints, subjects in Group B will also be assessed by the following endpoint:
Minimal Residual disease (MRD) status: The proportion of subjects in post-autologous transplant population that converted from MRD positive to MRD negative.

Secondary endpoints also include summary of select PK parameters, such as Cmax, AUC (TAU) and CLT based on concentration time data obtained from urelumab during the induction phase of treatment. In addition, Cmax and Cmin are captured at steady state for urelumab and elotuzumab based on the concentration time data from in the maintenance phase.

The concentration data obtained in this study may be combined with data from other studies in the clinical development program to develop or refine a population PK mode. This model can be used to evaluate the effects of intrinsic and extrinsic covariates on the PK of urelumab and elotuzumab to determine measures of individual exposure. In addition, model determined exposures can be used for exposure-response analyses.

Immunogenicity of urelumab and elotuzumab are reported for ADA positive status (such as persistent positive, transient positive, only last sample positive, baseline positive) and ADA negative status, relative to baseline. In addition, presence of neutralizing antibodies is reported, if applicable. Effect of immunogenicity on safety are explored if there is sufficient number of subjects with persistent positive ADA.

Biomarkers: Measures of NK, T, and Plasma cell number and phenotype are determined using flow cytometry on serial bone marrow aspirate samples and peripheral blood samples from all patients, and measures of soluble factors.

### 11. Analyses

Unless otherwise specified, safety data is summarized: 1) overall, across dose escalation and cohort expansion by dose level, and 2) overall and by treatment group (A or B) in cohort expansion. Efficacy data is summarized for each arm by treatment group in cohort expansion.

All subjects who receive study drug therapy are included in the analysis of safety endpoints. All recorded AEs are listed and tabulated by system organ class, preferred term, relationship to study drug, and treatment. Coding is performed according to the most current version of MedDRA. Vital signs and select clinical laboratory tests results are listed and summarized by treatment. Any significant physical examination finding and results of clinical laboratory tests are listed. Any electrocardiogram (ECG) abnormalities identified are listed.

Efficacy is listed for subjects in dose escalation and summarized by treatment group in cohort expansion. The decision to do this is made, because not all efficacy endpoints are relevant for all treatment groups. Summary of escalation data is provided by dose level and treatment group for subjects in escalation who meet criteria for one of the treatment groups in cohort expansion. Relevant endpoints vary by treatment group in cohort expansion.

The landmark progression free survival rate and corresponding 95% confidence intervals are estimated at preselected timepoints using Kaplan-Meier methodology. In addition, the Kaplan-Meier plots are generated by treatment group in cohort expansion. Objective response rate (e.g., CR+PR), the rate of conversion from minimal residual disease positive to minimal residual disease negative, and the rate of CR responses are tabulated; exact binomial 95% confidence intervals are provided using the clopper-pearson method. The distribution of the raw values and change from baseline in m-protein levels are summarized at each timepoint using descriptive statistics. Spider plots depicting changes in tumor burden over time can be generated for patients with measurable disease. In addition, plots can be produced showing m-protein levels as a function of time. Depending on the purpose of the analysis, response can reported for all treated subjects, or for response-evaluable subjects. The 1 and 2 year overall survival rates re evaluated using Kaplan-Meier methodology in subjects in the smoldering treatment group of expansion.

The pharmacodynamic effect on immune cell number and function is assessed by summary statistics and plots. In addition, the correlation of bone marrow immune cell number and function with measures of peripheral blood markers is explored graphically, or by appropriate statistical methods based on data availability, for assessing associations. The pharmacodynamic effect of treatment on markers in peripheral blood and serum proteins is assessed by summary statistics, and investigated graphically to explore patterns of change over time, and how the patterns differ among dose levels and exposure. If there is a meaningful indication in the pattern over time, further analysis (e.g., by linear mixed model) can be performed to characterize the relationship. Associations between biomarker measures from peripheral blood or bone marrow aspirate and clinical outcomes are explored graphically, and further assessed as needed by methods such as, but not limited to, logistic regression, and characterized by appropriate statistics.

### 12. Human Dose Projections

Three different approaches were utilized to predict the clinically efficacious BMS-663513 dose:

The first approach was “dose based”. Because 1 mg/kg in mice was identified as the minimally efficacious dose, this dose was normalized (surface area and body weight normalization) to identify the human dose of 0.08 mg/kg (i.e., ˜6 mg).

The second approach employed estimates of the “efficacious AUC”, which assumed that the minimum AUC required for efficacy in humans was 1950 ug/mL*hr (the minimally efficacious BMS-469492 AUC in the mouse). In the absence of extensive pharmacokinetic data in multiple species, it was assumed that the systemic clearance (˜0.0023 mL/min/kg) and Vss (˜58 mL/kg) values of BMS-663513 in monkeys and humans were similar. Dose is related to Cl and AUC (Dose=Cl*AUC), so the efficacious dose in humans was estimated to be ˜0.27 mg/kg (i.e., ˜20 mg).

The third approach was based on the calculation of a “minimum trough concentration”. The human pharmacokinetic parameters and plasma concentration-time profile were simulated using the animal data. In mice receiving the minimally efficacious dose (1.0 mg/kg) of BMS-469492, the trough concentration of BMS-469492 at 168 hr was 3.3 mg/mL (Table 13). Therefore, it was assumed that serum BMS-663513 concentrations ˜4 mg/mL, or higher, are needed to maintain efficacy in humans. The human dose was calculated such that the trough concentration at 168 hrs (1 week post-dose) was ˜4 mg/mL. The efficacious human dose was estimated to be 0.42 mg/kg (i.e., ˜30 mg).

Therefore, the efficacious human dose for BMS-663513 was estimated to be in the range of 0.08-0.42 mg/kg. This represents a single dose of 6-30 mg per week. However, upon review of the tolerability, efficacy, and PK observed in data from the CA186001 and CA186006 monotherapy studies of Urelumab, a dose of 0.1 mg/kg and 0.3 mg/kg, as well as 3 mg (corresponding to a dose of 0.03 mg/kg for a 80 kg human) and 8 mg (corresponding to a dose of 0.01 mg/kg for an 80 kg human) was selected for use in the current study in combination with Elotuzumab.

The entire disclosure of each document cited (including patents, patent applications, journal articles, abstracts, laboratory manuals, books, GENBANK® Accession numbers, SWISS-PROT® Accession numbers, or other disclosures) in the Background of the Invention, Detailed Description, Brief Description of the Figures, and Examples is hereby incorporated herein by reference in their entirety. Further, the hard copy of the Sequence Listing submitted herewith, in addition to its corresponding Computer Readable Form, are incorporated herein by reference in their entireties.

The present invention is not to be limited in scope by the embodiments disclosed herein, which are intended as single illustrations of individual aspects of the invention, and any that are functionally equivalent are within the scope of the invention. Various modifications to the models and methods of the invention, in addition to those described herein, will become apparent to those skilled in the art from the foregoing description and teachings, and are similarly intended to fall within the scope of the invention. Such modifications or other embodiments can be practiced without departing from the true scope and spirit of the invention.

What is claimed is:

1. A method for treating a patient with cancer comprising the concurrent administration of a combination therapeutic regiment comprising: (i) a therapeutically effective amount of an agonistic CD137 antibody; and (ii) a therapeutically effective amount of an anti-CS1 antibody, wherein said combination results in the synergistic reduction in tumor burden, tumor regression, and/or tumor development of said cancer.

2. The method of claim 1, wherein said cancer is selected from the group consisting of: myeloma, multiple myeloma, and smoldering myeloma.

3. The method according to claim 1, wherein said agonistic CD137 antibody is urelumab.

4. The method of claim 1, 2, or 3, wherein said anti-CS1 antibody is elotuzumab.

5. The method of claim 1, wherein said agonistic CD137 antibody is administered at a dosage of about 0.1-1 mg/kg, and said anti-CS1 antibody is administered at a dosage of about 0.1-1 mg/kg once every three weeks.

6. The method of claim 1, wherein said agonistic CD137 antibody is administered at a dosage of about 0.1-1 mg/kg, and said anti-CS1 antibody is administered at a dosage of about 1 mg/kg once every three weeks.

7. The method of claim 1, wherein said agonistic CD137 antibody is administered at a dosage of about 0.1-1 mg/kg, and said anti-CS1 antibody is administered at a dosage of about 10 mg/kg once every three weeks.

8. The method of claim 1, wherein said agonistic CD137 antibody is administered at a dosage of about 0.03-0.1 mg/kg or about 3 mg-8 mg, and said anti-CS1 antibody is administered at a dosage of about 1 mg/kg or 10 mg/kg once every three weeks.

9. A method for treating a patient with cancer comprising the sequential administration of a combination therapeutic regiment comprising: (i) a therapeutically effective amount of an anti-CS1 antibody; followed by (ii), the a therapeutically effective amount of an agonistic CD137 antibody, wherein said combination results in the synergistic reduction in tumor burden, tumor regression, and/or tumor development of said cancer.

10. The method of claim 8, wherein said cancer is selected from the group consisting of: myeloma, multiple myeloma, and smoldering myeloma.

11. The method according to claim 8, wherein said agonistic CD137 antibody is urelumab.

12. The method of claim 8, 9, or 10, wherein said anti-CS1 antibody is elotuzumab.

13. The method of claim 8, wherein said agonistic CD137 antibody is administered at a dosage of about 0.1-1 mg/kg, and said anti-CS1 antibody is administered at a dosage of about 10 mg/kg once every three weeks.

14. The method of claim 8, wherein said agonistic CD137 antibody is administered at a dosage of about 0.03-0.1 mg/kg or 3 mg-8 mg, and said anti-CS1 antibody is administered at a dosage of about 1 mg/kg or 10 mg/kg once every three weeks.

15. A method of treating multiple myeloma in a human patient, the method comprising administering to the patient an effective amount of each of:
(a) an agonistic CD137 comprising the CDR1, CDR2 and CDR3 domains in a heavy chain variable region comprising the sequence set forth in SEQ ID NO:4, and the CDR1, CDR2 and CDR3 domains in a light chain variable region comprising the sequence set forth in SEQ ID NO:3,
(b) an anti-CS1 antibody comprising the CDR1, CDR2 and CDR3 domains in a heavy chain variable region comprising the sequence set forth in SEQ ID NO:2, and the CDR1, CDR2 and CDR3 domains in a light chain variable region comprising the sequence set forth in SEQ ID NO:1,
wherein (A) the anti-CS1 antibody is administered weekly for a total of 8 doses over 8 weeks and the agonistic CD137 is administered every 4 weeks for a total of 2 doses over 8 weeks during an induction phase, and
wherein the agonistic CD137 is administered at a dose of 0.03-0.1 mg/kg body weight and the anti-CS1 antibody is administered at a dose of 0.1-20 mg/kg body weight during both the induction and maintenance phases.

16. A method of treating multiple myeloma in a human patient, the method comprising administering to the patient an effective amount of each of:
(a) an agonistic CD137 comprising the CDR1, CDR2 and CDR3 domains in a heavy chain variable region comprising the sequence set forth in SEQ ID NO:4, and the CDR1, CDR2 and CDR3 domains in a light chain variable region comprising the sequence set forth in SEQ ID NO:3,
(b) an anti-CS1 antibody comprising the CDR1, CDR2 and CDR3 domains in a heavy chain variable region comprising the sequence set forth in SEQ ID NO:2, and the CDR1, CDR2 and CDR3 domains in a light chain variable region comprising the sequence set forth in SEQ ID NO:1,
wherein (A) the anti-CS1 antibody is administered weekly for a total of 8 doses over 8 weeks and the agonistic CD137 is administered every 4 weeks for a total of 2 doses over 8 weeks during an induction phase, followed by (B) administration of the anti-CS1 antibody every 2 weeks and administration of the agonistic CD137 every 4 weeks during a maintenance phase, and
wherein the agonistic CD137 is administered at a dose of 0.03-1 mg/kg body weight and the anti-CS1 antibody is administered at a dose of 0.1-20 mg/kg body weight during both the induction and maintenance phases.


SEQUENCE LISTING (8 sequences)

SEQ ID NO: 1 - protein, 107 aa, Homo sapiens
DIQMTQSPSSLSASVGDRVTITCKASQDVGIAVAWYQQKPGKVPKLLIYWASTRHTGVPDRFSGSGSGTDFTLTISSLQPEDVATYYCQQYSSYPYTFGQGTKVEIK

SEQ ID NO: 2 - protein, 119 aa, Homo sapiens
EVQLVESGGGLVQPGGSLRLSCAASGFDFSRYWMSWVRQAPGKGLEWIGEINPDSSTINYAPSLKDKFIISRDNAKNSLYLQMNSLRAEDTAVYYCARPDGNYWYFDVWGQGTLVTVSS

SEQ ID NO: 3 - protein, 236 aa, Homo sapiens
MEAPAQLLFLLLLWLPDTTGEIVLTQSPATLSLSPGERATLSCRASQSVSSYLAWYQQKPGQAPRLLIYDASNRATGIPARFSGSGSGTDFTLTISSLEPEDFAVYYCQQRSNWPPALTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 4 - protein, 467 aa, Homo sapiens [sequence omitted]

SEQ ID NO: 5 - protein, 9 aa, Homo sapiens
IMDQVPFSV

SEQ ID NO: 6 - protein, 9 aa, Homo sapiens
YLEPGPVTV

SEQ ID NO: 7 - protein, 334 aa, Homo sapiens
MAGSPTCLTLIYILWQLTGSAASGPVKELVGSVGGAVTFPLKSKVKQVDSIVWTFNTTPLVTIQPEGGTIIVTQNRNRERVDFPDGGYSLKLSKLKKNDSGIYYVGIYSSSLQQPSTQEYVLHVYEHLSKPKVTMGLQSNKNGTCVTNLTCCMEHGEEDVIYTWKALGQAANESHNGSILPISWRWGESDMTFICVARNPVSRNFSSPILARKLCEGAADDPDSSMVLLCLLLVPLLLSLFVLGLFLWFLKRERQEEYIEEKKRVDICRETPNICPHSGENTEYDTIPHTNRTILKEDPANTVYSTVEIPKKMENPHSLLTMPDTPRLFAYENV

SEQ ID NO: 8 - protein, 255 aa, Homo sapiens
MGNSCYNIVATLLLVLNFERTRSLQDPCSNCPAGTFCDNNRNQICSPCPPNSFSSAGGQRTCDICRQCKGVFRTRKECSSTSNAECDCTPGFHCLGAGCSMCEQDCKQGQELTKKGCKDCCFGTFNDQKRGICRPWTNCSLDGKSVLVNGTKERDVVCGPSPADLSPGASSVTPPAPAREPGHSPQIISFFLALTSTALLFLLFFLTLRFSVVKRGRKKLLYIFKQPFMRPVQTTQEEDGCSCRFPEEEEGGCEL